(19) United States
(12) Patent Application Publication
(10) Pub. No.: US 2022/0125754 A1
(43) Pub. Date: Apr. 28, 2022

(54) TREATING THE CAUSATIVE AGENT IN ADHESIOGENESIS

(71) Applicant: Helmholtz Zentrum München - Deutsches Forschungszentrum Für Gesundheit Und Umwelt (GmbH), Neuherberg (DE)

(72) Inventors: Adrian Fischer, Munich (DE); Yuval Rinkevich, Munich (DE); Tim Koopmans, Houten (DE)

(21) Appl. No.: 17/425,687

(22) PCT Filed: Jan. 29, 2020
(86) PCT No.: PCT/EP2020/052150

§ 371 Date: Jul. 23, 2021

(30) Foreign Application Priority Data
Jan. 29, 2019 (LU) LU101108

(51) Int. Cl. A61K 31/352; A61P 43/00; G01N 33/50; A61K 31/498; A61K 31/554; A61K 31/277; A61K 31/4422; A61K 31/4025

(52) U.S. Cl. CPC A61K 31/352; A61P 43/00; G01N 33/50; A61K 31/4025; A61K 31/554; A61K 31/277; A61K 31/4422; A61K 31/498

(57) ABSTRACT

The present invention relates to a compound for use in a method of reducing the formation of heliocytes causing adhesiogenesis. An in vitro assay for the formation of heliocyte and/or the formation of adhesions is also comprised herein, as well as methods comprising the use of said in vitro assay. It also relates to a pharmaceutical composition for use in a method of reducing the formation of heliocytes comprising the compound mentioned above.

This application contains a Sequence Listing in computer readable form, which is incorporated herein by reference.

### TECHNICAL FIELD OF THE INVENTION

The present invention relates to a compound for use in a method of reducing the formation of heliocytes causing adhesiogenesis. An in vitro assay for the formation of heliocyte and/or the formation of adhesions is also comprised herein, as well as methods based upon the use of said in vitro assay. It also relates to a pharmaceutical composition for use in a method of reducing the formation of heliocytes comprising the compound mentioned above.

### BACKGROUND ART

Adhesions are the (pathological) seaming of organ surfaces with one another or with the walls of the cavity they reside in. It is the most common side-effect of trauma during abdominal surgery that stems from tissue mishandling, ischemia at incision sites, introduction to foreign bodies (e.g. talcum powder), or tissue desiccation^{1,2}. In addition to trauma, adhesions can develop from inflammatory processes, infections, or in response to dialysis fluid³. While adhesions habitually develop from injuries imposed to a single organ surface, the pathology subsequently expands to non-injured and adjacent surfaces, through a mechanism that remains obscure^{4,5}. The ramifications of adhesions range from pelvic pain and obstruction of organ movement, to severe organ failure. Adhesions that develop in the ovaries or fallopian tubes, after tumor removal or oophorectomy, are responsible for 15-20% of female infertility cases⁶. Over 90% of abdominal surgeries result in adhesions, a third of which end in re-hospitalization within 10 years^{7,8}. In the clinic, severe adhesions after surgery are mainly treated with re-operating in order to separate adhered organs, in an iatrogenic procedure that creates an enormous healthcare burden, estimated at over $1 billion a year in the United States alone. While the mechanisms leading injured organ surfaces to seam remains undisclosed, the mature stages of the adhesion process, including the activation of immune responses and the formation of macroscopic scars between fused surfaces are well documented and studied.

All visceral organs and their cavities are lined by an epithelial monolayer called the mesothelium that forms smooth and frictionless interfaces between adjacent organs, conferring protection to organ surfaces from abrasions and damage⁹. The current model, set by Schade and Williamson in 1968, proposes the removal of mesothelial cells from organ surfaces as the critical event in generating adhesions, where the exposed underlying basement membranes and deposition of fibrin allow migrating fibroblasts from the organ interiors to generate seams of fibrous bonds on organ surfaces¹⁰. However, substantial evidence for this model has been lacking, and several studies support adhesion formation with intact mesothelial layers.

A significant lack of model assays to study organ surface interactions have thus far hindered detailed insight into the pathomechanisms driving the early stages of adhesions. Additionally, specific compounds being used in the clinic which might have an impact on the early stages of adhesions are also missing.

Thus, the objective of the present invention is to comply with this need.

### SUMMARY OF THE INVENTION

The inventors investigated organ adhesions at the single cell level by coating microcarrier beads with human mesothelial cells and subjecting said cells to e.g. desiccation or foreign body exposure (which are also called activation stimuli), two clinical risk factors for developing adhesions.

The inventors then observed that said activation stimuli induced massive membrane protrusions from the mesothelium that fused beads to form adhesion foci in a matter that closely resembles physiological adhesions. These membrane protrusions intimately contact adjacent cell surfaces, further triggering new protrusions that propagate and extend the adhesion pathology. Fusions between organ surfaces develop by these protrusions.

The inventors further observed that inactivated (also called unstressed) mesothelial cells maintained a typical mesothelial cobblestone appearance and lacked any membrane extensions. Due to their dramatic shift in morphology and close resemblance to microbial heliozoa, the inventors called the activated (also called stressed) cells inducing membrane protrusions ‘heliocytes’ These observations were only possible for the inventors to investigate by applying their own in vitro bead assay, which certainly and greatly facilitated exploration of early adhesion events.

So far, the prior art in general focuses on preventing adhesiogenesis. In particular, WO2017/190148 determines injured mesothelial cells as a primary cell type responsible for contributing towards adhesion. Accordingly, '148 application discloses and teaches to use an agent that targets adhesion-formation by injured mesothelial cells. The '148 application induces adhesion by placing so-called ischemic buttons in mice. Hence, when the inventors of said prior art document harvested such tissue/cells from mice, they could only observe the formation of adhesions, i.e., the symptoms. However, the '148 application did not understand the exact mode-of-action that occurs when mesothelial cells may be stressed, or injured. Hence, the '148 application only teaches targeting “adhesion-formation by injured mesothelial cells” and thus treating the “symptoms” (adhesion formation), while the present invention are in a position to treat the “causative agent” (formation of heliocytes), since the '148 application had neither an idea nor a test available which allowed them to observe in a nascent state mesothelial cells becoming such heliocytes.

The present invention however used “naïve”, i.e., not induced mesothelial cells and coated them on microcarrier beads, then inducing the adhesion of the cell-coated beads by activation. By that, the inventors were able to see and understand the mode-of-action and thereby unveiling the conversion of mesothelial cells into heliocytes which develop membrane protrusions described above and then later on develop adhesions. Only based on the observation of the surface to surface interaction of stressed mesothelium cells in the microscale bead assay of the inventors, the mode-of action of adhesion (adhesiogenesis) could be investigated. Investigating only a monolayer of mesothelial cells, which had been studied in the '148 application, may not be sufficient to clearly observe the formation of heliocytes, since at least one 2D surface fusing with another 2D surface of cells (surface-to-surface interaction of cells) may be required for the exploration of this early adhesion event.

The present invention has provided a new clinical situation due to enabling the treatment of the formation of heliocytes which are the cause of the symptom, i.e., adhesions. Thus, the present invention provides an enabling technology which is the tool to help the skilled person to directly treat the causative agent (reducing the formation of heliocytes) rather than indirectly treating the already occurred adhesion-formation.

Such reduction of the formation of heliocytes may be performed by using the specific compounds of the present invention. Thus in a first aspect the present invention relates to a compound for use in a method of reducing heliocytes, wherein a heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 or a phosphorylated Myosin 9 light chain on protein level.

Therefore, the present invention deals with a compound for use in a method of reducing the formation of said specific cells called “heliocytes”.

Further, the present invention may envisage the compound for the use as described above, wherein a mesothelial cell is activated by hypoxia, ischemia, inflammation, infection, a chemical stimulus, desiccation, a mechanical trauma, cold shock, heat shock, osmotic shock, or a foreign body to become an activated mesothelial cell.

Further comprised herein may be the compound for the use as mentioned above, wherein a heliocyte is characterized by membrane protrusions of the akropodia-type, or membrane protrusions of the filopodia-type.

Additionally, the present invention may comprise the compound for the use according to the abovementioned, wherein a heliocyte is characterized by vesicle and/or exosome secretion.

The present invention may further comprise the compound for the use as mentioned above, wherein heliocytes develop adhesions. The present invention may further comprise the compound for the use as mentioned above, wherein the development of adhesions by heliocytes results in adhesiogenesis, preferably wherein adhesiogenesis is inter- or intra-organ adhesiogenesis and wherein said adhesiogenesis occurs postoperative.

The present invention may further comprise the compound for the use as mentioned above, wherein the compound is capable of preventing the transmission of a mesothelial cell to the pathogenic phenotype of a heliocyte and/or capable of inducing apoptosis in a heliocyte, but not in a mesothelial cell.

Additionally, the present invention may further comprise the compound for the use as mentioned above, wherein the compound blocks cytoskeletal remodeling, blocks protein trafficking, blocks calcium signaling or blocks heat shock protein signaling.

Also envisaged by the present invention, may be the compound for the use as mentioned above wherein the compound is selected from the group consisting of Bepridil, Verapamil, Diltiazem, Nifedipine, Rhosin, CK-666, Golgicide A, KNK437, and Quercetin. Said compound for the use as mentioned above may further be combined with a heat shock protein signaling blocker, preferably Quercetin. Said compound for the use as mentioned above may be a calcium channel blocker selected from the group consisting of Diltiazem, Verapamil, Nifedipine and Bepridil. Further, said compound for the use as mentioned above may be a heat shock protein signaling blocker selected from the group consisting of KNK437, and Quercetin. Said compound for the use as mentioned above may also be a cytoskeletal remodeling blocker selected from the group consisting of Rhosin, and CK-666.

Additionally, the present invention may further comprise the compound for the use as mentioned above, wherein the use may further comprise administering the compound as defined herein after surgery or injury, determining the adhesion formation by heliocytes and continuing the compound treatment if the adhesion formation by heliocytes decreased as compared to the pre-treatment.

In another aspect the present invention relates to an in vitro bead assay for analyzing heliocytes and/or the formation of adhesions, which comprises the steps of

a) seeding mesothelial cells onto a coated dish and letting said cells grow to a monolayer;
b) coating carrier beads with mesothelial cells;
c) activating said cells coated on said carrier beads of step b) with a stimulus,
d) seeding said activated cells coated on said carrier beads of step b) and c) onto the monolayer of step a); and analyzing the activated mesothelial cells on said carrier beads, or analyzing the activated mesothelial cells eluted from said carrier beads.

In another embodiment the in vitro bead assay for analyzing heliocytes and/or the formation of adhesions, comprises mesothelial cells which may preferably be Met-5A positive, before being seeded in step a) and/or coated in step b), and furthermore the activated cells on said carrier beads of step c) and/or step d) may be capable of fusing the cell-coated beads together and may optionally be selected by size. Also comprised herein, is said in vitro bead assay for the use as mentioned above, wherein the stimulus of step c) may be selected from the group consisting of hypoxia, ischemia, inflammation, infection, a chemical stimulus, desiccation, a mechanical trauma, cold shock, heat shock, osmotic shock, or a foreign body.

Further comprised herein may be the in vitro bead assay for analyzing heliocytes and/or the formation of adhesions, which may further comprise

i) contacting said activated cells coated on said carrier beads after step c) with a compound; and
ii) determining the effect of the compound on the activated mesothelial cells and/or formation of adhesions after step d).

Additionally, the present invention may comprise the compound for the use as described herein, wherein said capability of said compound is determined by the in vitro bead assay for analyzing heliocytes and/or the formation of adhesions.

Also envisaged by the present invention, may be the in vitro bead assay for the use as mentioned above preferably for determining the capability of a compound to a) prevent the transmission of a mesothelial cell to the pathogenic phenotype of a heliocyte; and/or b) induce apoptosis in a heliocyte, but not in a mesothelial cell; and/or c) prevent the formation of adhesion and/or adhesiogenesis.

In another aspect the present invention relates to an in vitro method for determining the formation of heliocytes, wherein the method comprises obtaining a sample comprising mesothelial cells from a subject, preparing the sample according to the in vitro bead assay for analyzing heliocytes and/or the formation of adhesions and determining the formation of heliocytes in said in vitro bead assay.

In another aspect the present invention relates to an in vitro method for treating heliocytes and/or adhesions formed by heliocytes, wherein the method comprises obtaining a sample comprising mesothelial cells from a subject, preparing the sample according to the in vitro bead assay for analyzing heliocytes and/or the formation of adhesions and treating the heliocytes and/or adhesions formed by heliocytes by contacting said heliocytes with a compound according to the in vitro bead assay.

In another aspect the present invention relates to a calcium channel blocker for use in a method of reducing adhesions formed by heliocytes as defined somewhere else herein. The calcium channel blocker for the use as mentioned above may be Diltiazem, Verapamil, Nifedipine and Bepridil, preferably Diltiazem, Verapamil, Bepridil. Further comprised herein may be said calcium channel blocker for the use as mentioned above, wherein the method may comprise a) administering to a subject an effective amount of calcium channel blocker to prevent the formation of adhesion by heliocytes after surgery or injury; b) determining the formation of adhesion by heliocytes after the treatment with said calcium channel blocker; c) continuing the treatment if the heliocytes and/or adhesion formation by heliocytes decreased as compared to the pre-treatment.

In another aspect the present invention relates to a heat shock protein signaling blocker for use in a method of reducing adhesions formed by heliocytes as defined somewhere else herein. The heat shock protein signaling blocker for the use mentioned above may be KNK437, or Quercetin, preferably KNK437. Further comprised herein may be said heat shock protein signaling blocker for the use as mentioned above, wherein the method may comprise a) administering to a subject an effective amount of heat shock protein signaling blocker to prevent the formation of adhesion by heliocytes after surgery or injury; b) determining the formation of adhesion by heliocytes after the treatment with said heat shock protein signaling blocker; c) continuing the treatment if the heliocytes and/or adhesion formation by heliocytes decreased as compared to the pre-treatment.

In another aspect the present invention relates to a cytoskeletal remodeling blocker for use in a method of reducing adhesions formed by heliocytes, as defined somewhere else herein. The cytoskeletal remodeling blocker for the use as mentioned above may be Rhosin, or CK-666, preferably Rhosin. Further comprised herein may be said cytoskeletal remodeling blocker for use as mentioned above, wherein the method may comprise a) administering to a subject an effective amount of cytoskeletal remodeling blocker to prevent the formation of adhesion by heliocytes after surgery or injury; b) determining the formation of adhesion by heliocytes after the treatment with said cytoskeletal remodeling blocker; c) continuing the treatment if the heliocytes and/or adhesion formation by heliocytes decreased as compared to the pre-treatment.

In another aspect the present invention relates to a pharmaceutical composition for use in a method of reducing the formation of heliocytes, comprises at least one compound(s) as defined herein and one or more pharmaceutically acceptable excipients.

In another aspect the present invention relates to an in vitro method for detecting the presence of heliocytes forming adhesions in a subject, comprises: a) providing a sample obtained from a subject, said sample comprising one or more cell(s); b) seeding a plurality of cells of a subject in the in vitro bead assay for analyzing heliocytes and/or the formation of adhesions; c) contacting said cells with i) the compound as described herein and/or, ii) the pharmaceutical composition as described herein; and c) detecting the presence of heliocytes forming adhesions in the cells seeded in said in vitro bead assay, wherein the detection of heliocytes forming adhesions is indicative of heliocytes forming adhesions in the subject.

In another aspect the present invention relates to a method of selecting a subject for calcium channel blocker treatment, comprises a) determining the heliocyte formation in a sample, wherein the sample has been obtained from a subject prior to calcium channel blocker treatment; b) determining the heliocyte formation in a sample, wherein the sample has been obtained from the subject after treatment with the calcium channel blocker; c) selecting the subject for continuing the calcium channel blocker treatment if the heliocyte formation is decreased in step c) as compared to step a).

In another aspect the present invention relates to a method of selecting a subject for heat shock protein signaling blocker treatment, comprises a) determining the heliocyte formation in a sample, wherein the sample has been obtained from a subject prior to heat shock protein signaling blocker treatment; b) determining the heliocyte formation in a sample, wherein the sample has been obtained from the subject after treatment with the heat shock protein signaling blocker; c) selecting the subject for continuing the heat shock protein signaling blocker treatment if the heliocyte formation is decreased in step c) as compared to step a).

In another aspect the present invention relates to a method of selecting a subject for cytoskeletal remodeling blocker treatment, comprises a) determining the heliocyte formation in a sample, wherein the sample has been obtained from a subject prior to cytoskeletal remodeling blocker treatment; b) determining the heliocyte formation in a sample, wherein the sample has been obtained from the subject after treatment with the cytoskeletal remodeling blocker; c) selecting the subject for continuing the cytoskeletal remodeling blocker treatment if the heliocyte formation is decreased in step c) as compared to step a).

### BRIEF DESCRIPTION OF THE FIGURES

FIG. 1: Microcarrier model recapitulates physiological adhesions. A) Overview of the in vitro bead assay. B) Different stress setups for adhesion formation: 1) desiccation 2) 10 μg/ml talcum 3) cold shock: 10 minutes at −20° C. 4) heat shock: 10 minutes at 42° C. Each of the setups induces adhesion formation in vitro using the bead assay. C) and D) Desiccation shock induces carrier-carrier aggregation, which develop as fast as 60 min after injury. E) Scanning electron microscopy image of clustered beads; Scale bar, 50 μm.

FIG. 2: Microcarrier clustering as a model for adhesions. A) Overview of the high-throughput nanoluciferase bead assay. B) and C) Desiccation shock and talcum exposure both induce carrier-carrier aggregation. D) Overview of the experimental approach of the mixture of stressed luciferase-expressing carriers with unstressed nanoluciferase-expressing carriers. E) Nanoluciferase emission of unstressed nanoluciferase carriers vs. stressed carriers. Stressed carriers actively incorporated unstressed nanoluciferase carriers into large carrier aggregates, indicating adhesions can spread to healthy surfaces. F) Scanning electron microscopy image of clustered beads. Scale bar, 50 μm. G) Phase-contrast image and corresponding silhouette of stressed and unstressed Met-5A cells seeded on a Matrigel bedding. H) Spinning disc data showing required forces to detach carrier clusters, various time points after desiccation shock. I) Spinning disc data as in (H) of carrier-to-monolayer attachments

FIG. 3: Heliocytes produce cytoskeletal protrusions to bind and transmit adhesive potential. A) Stable cell line expressing membrane-bound GFP show dramatic change in morphology after desiccation shock. Scale bar, 20 μm. B) and C) Machine learning algorithm based on the ‘advanced weka segmentation’ Fiji plugin, employed to detect the total filopodial area in heliocytes. D) Measured characteristics of different protrusions present on heliocytes. E) Total surface area of main and akropodial cell body, performed as in (B). F, G and H) Adhesion propagation assay (see methods) with nanoluciferase expressing Met-5A cells. I) Carrier-monolayer confocal image after successful Cre-recombinase transmission. Scale bar, 50 μm. J) and K) Cre-exchange transmission assay (see methods) with Cre-recombinase and Cre-dependent nanoluciferase expressing Met-5A cells.

FIG. 4: Force distribution and calcium signaling in heliocytes. A) Representative image of filopodial (small—magenta, large—green) recognition using machine learning, derived from a fluorescent image of a Met-5A cell stably expressing membranous GFP. B) Fluorescent image (top view) of carrier-to-monolayer Met-5A cells expressing the calcium indicator GCaMP6s. Scale bar, 150 μm. C) Fluorescent image (top view) of carrier-to-monolayer Met-5A cells labelled with the calcium reporter X-Rhod-1. Scale bar, 100 μm. D) Confocal image of whole-mount stain of IP3R in stressed and unstressed beads 2 days after hypoxic shock. Scale bar, 50 μm.

FIG. 5: Single-cell RNAseq identifies cytoskeletal effectors as core heliocyte program. A) Principal component analysis of 1644 genes showing time differences after desiccation shock. B) Volcano plot of top regulated genes 8 hours after desiccation shock. C) Violin plots showing normalized expression (scored by UMIs) of different time points after desiccation shock. D) Confocal image of whole-mount stains of carrier-carrier complexes 2 days after desiccation shock. Scale bar, 40 μm.

FIG. 6: Single-cell transcriptomics of heliocytes. A) Overview of the Dropseq workflow. B) Epi-fluorescent representative images of Met-5A cells stably expressing membranous GFP, stressed with desiccation and treated with small-molecule compounds for 24 hours (10 μM). Scale bar, 10 μm. C) Desiccation shock induces small-molecule sensitive carrier-carrier aggregation as assessed with a high-throughput nanoluciferase assay. ID) Adhesion propagation assay (see methods) with nanoluciferase expressing Met-5A cells after treatment with small molecule inhibitors for 24 hours (10 μM). E) Cre-exchange transmission assay (see methods) with Cre-recombinase and Cre-dependent nanoluciferase expressing Met-5A cells after treatment with small molecule inhibitors for 24 hours (10 μM).

FIG. 7: Targeted ablation of cytoskeletal effectors prevents adhesion formation in vivo. A) Confocal image of whole-mount healthy and injured pMYL9+ and PDPN+ peritoneum. Scale bar, 30 μm. B) Adhesion score (see methods) 5 days after injury, of mice treated with small-molecule compounds dissolved in 2% cellulose that was applied topically at the injury site.

FIG. 8: Heliocyte profiling in murine adhesions. A), B) and C) Confocal images of whole-mount injured PDPN+, pan Rho+, AKAP12+, and ARF-GAP1+ peritoneum 16 hours after injury. Scale bar, 40 μm.

FIG. 9: Small-molecule inhibition of heliocyte programs prevents adhesion development. A) Adhesion score (see methods) 5 days after injury, of mice treated with small-molecule compounds injected intraperitoneally daily.

FIG. 10: Proposed model for the early events driving adhesiogenesis. Injury to a serosal layer induces a dramatic and rapid shift in mesothelial morphology through the formation of cytoskeletal protrusions. These allow for 1) the physical binding to neighbouring healthy cells (e.g. at apposing serosal surfaces), and 2) transmission of pathological behaviour. This initiates organ tethering and rapid spread of adhesions through serosal surfaces. Once established, heliocytes commit MMT and deposit matrix to form a macroscopic scar.

FIG. 11: Apoptosis Assay. Measurement of fragmented DNA as indicator for apoptosis after 5 days having applied CK666, Golgicide A, Rhosin, Bepridil, Heat shock protein inhibitor 1 (HIS-1) using the in vitro bead assay. Stautosporin serves as a positive control.

FIG. 12: Stressed mesothelial cells (heliocytes) stably expressing the reporter construct (nanoluciferase fused to a degradation signaling peptide interlinked with caspase3 cleavage sites) show enhanced nanoluciferase activity upon treatment with Staurosporine (positive control). Rhosin and heat shock protein inhibitor 1 demonstrating higher caspase3 activity in treated heliocytes compared to the control (Ctrl).

FIG. 13: Confocal images of heat shock protein (HSP) 27, 70 and 105 expression pattern in healthy and injured murine tissue. Scale bar, 40 μm.

FIG. 14: Treating mesothelial cells with vesicles derived from stressed mesothelial cells (heliocytes) in the in vitro bead assay induces adhesion formation without additional stress stimuli in comparison to vesicles derived from unstressed mesothelial cells.

FIG. 15: A) Adhesion score of tamoxifen-treated Procr-DTA mice 5 days after injury. B) Adhesion score 2 months after injury, of mice treated with small-molecule compounds dissolved in 2% cellulose that was applied topically at the injury site once before closure. C) Adhesion score 5 days after injury, of mice treated with small-molecule compounds dissolved in 0,9% NaCl applied via one shot injections after closure. D) Adhesion score 5 days after injury, of mice treated with calcium channel inhibitors dissolved in 2% cellulose that was applied topically at the injury site once before closure. E) Adhesion score 5 days after injury, of mice treated with heat shock factor inhibitors dissolved in 2% cellulose that was applied topically at the injury site once before closure. F) Adhesion score 5 days after injury, of mice treated with heat shock factor inhibitors dissolved in 2% cellulose that was applied orally 2 hours before injury. G) Adhesion score 5 days after injury, of mice treated small-molecule compounds dissolved in 2% cellulose that was applied topically at the injury site once before closure.

FIG. 16: Adhesion score 5 days after injury of mice treated with inhibitors Nifedipine and KRIBB11 dissolved in 2% cellulose that was applied topically at the injury site once before closure.

### DETAILED DESCRIPTION OF THE INVENTION

The solution of the present invention is described in the following, exemplified in the appended examples, illustrated in the figures and reflected in the claims.

### Purpose-Related Product

As described above, one aspect of the present invention is a compound for use in a method of reducing the formation of heliocytes.

A heliocyte in the context of the present invention may be an activated mesothelial cell.

Mesothelial cells are derived from the mesoderm but express both mesenchymal and epithelial cell intermediate filaments. Mesothelial cells form a monolayer of specialized pavement-like cells (called the mesothelium) lining the body's serosal cavities (pleural, pericardial and peritoneal) and internal organs contained within these cavities. The primary function of the mesothelium is to provide a slippery, non-adhesive and protective surface. Such cells are also involved in transport of fluid and cells across the serosal cavities, antigen presentation, inflammation and tissue repair, coagulation and fibrinolysis and tumor cell adhesion. Injury to the mesothelium triggers events leading to the migration of mesothelial cells from the edge of the lesion towards the wound center and desquamation of cells into the serosal fluid which attach and incorporate into the regenerating mesothelium.

A mesothelial cell of the present invention and as used throughout the entire description may be a mammalian mesothelial cell. A mesothelial cell of the present invention may also refer to a mesothelioma cell. A mammalian mesothelial cell may refer, but is not limited to a human mesothelial cell. A human mesothelial cell may be selected from the group consisting of human pleural mesothelial cell line Met-5A, and human primary mesothelial cells (HMTC). In a preferred embodiment, a human mesothelial cell is a Met-5A cell.

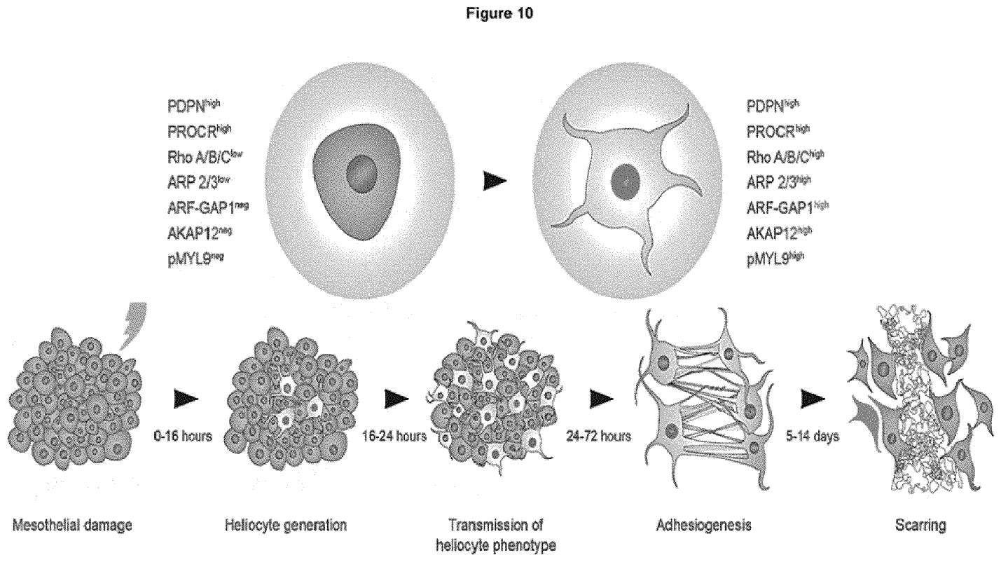

In this context and as used throughout the entire description, the term “activated” may also refer to the term “stressed”, meaning an activated mesothelial cell may also be a stressed mesothelial cell. The term “activated” as used throughout the entire description is not equal to an “injured” mesothelial cells because injured mesothelial cells may include necrotic or dead cells. However, an injury can cause the activation of mesothelial cells. The term “activated” mesothelial cell describes molecular reactions of said mesothelial cell to a defined stimulus, which may result ultimately in a change of cellular gene expression and/or morphological phenotype. FIG. 10 further illustrates the differences and development of mesothelial cells to heliocytes.

To find out what activation stimuli may lead to the activation of said mesothelial cells, the inventors tried to establish an in vitro model of organ fusions applying several stress stimuli to the mesothelial cells. In this context, the term “stress stimuli”, “stress indicators”, “activation indicators”, “adhesion stimuli” may be used interchangeably with the term “activation stimuli” or just “stimuli”.

Those different stimuli may mimic risk factors in vivo which lead to post-surgical adhesions. Post-surgical adhesions are primarily caused by three risk factors: 1) damage to organ surfaces due to surgical mishandling; 2) hypoxic pockets that develop at severed vessels and nerves; 3) talcum powder irritation from surgical gloves. Post-surgical adhesions may be understood as extensive scaring of the peritoneum/inner tissue. In fact, the major cause of bowel obstruction is adhesion formation following abdominal surgery. Adhesion formation after for instance pelvic surgery is common and may be associated with significant morbidity, including infertility, chronic pelvic pain, bowel obstruction, and difficult repeat operations. Thus, a reduction of adhesion formation after surgery and/or injury could prevent the symptoms of significant morbidity, including but not limited to infertility, chronic pelvic pain, bowel obstruction, and difficult repeat operations. Furthermore, a reduction in the formation of heliocytes which are activated mesothelial cells causing adhesiogenesis after surgery and/or injury include but not limited to prevent significant morbidity, infertility, chronic pelvic pain, bowel obstruction, and difficult repeat operations.

Thus, the compound as described herein used for the reduction of heliocytes and/or adhesiogenesis may ultimately reduce the negative effects of adhesion formation which include but are not limited to significant morbidity, including infertility, chronic pelvic pain, bowel obstruction, and difficult repeat operations.

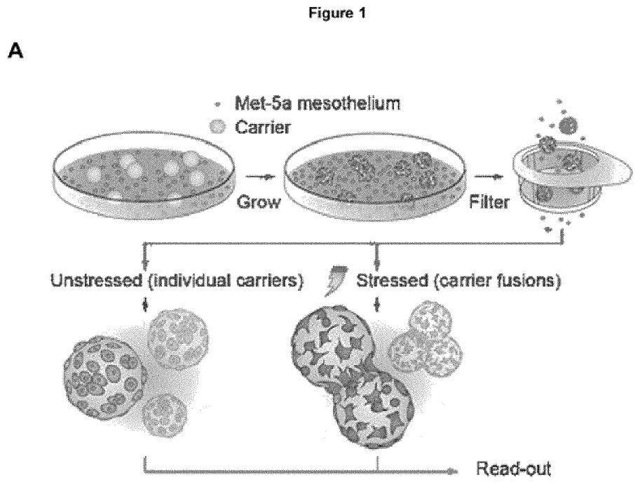

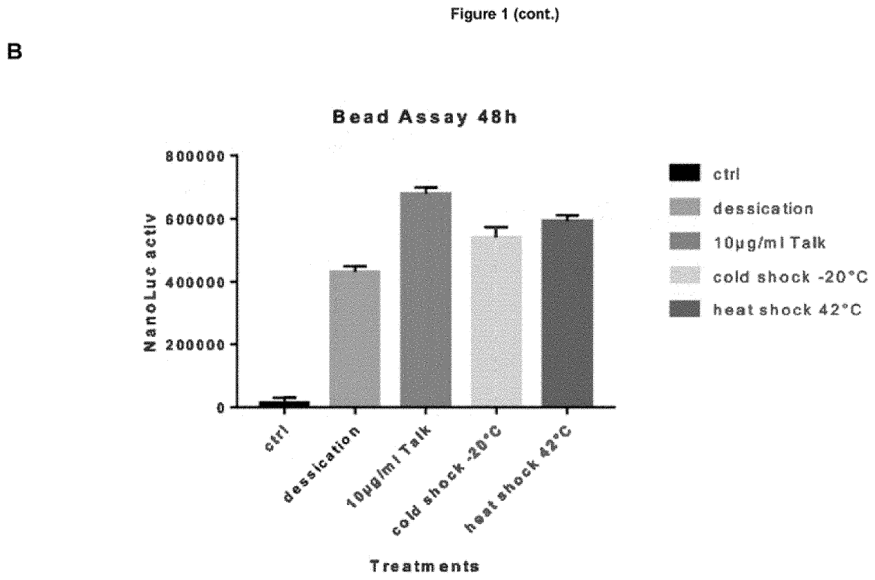

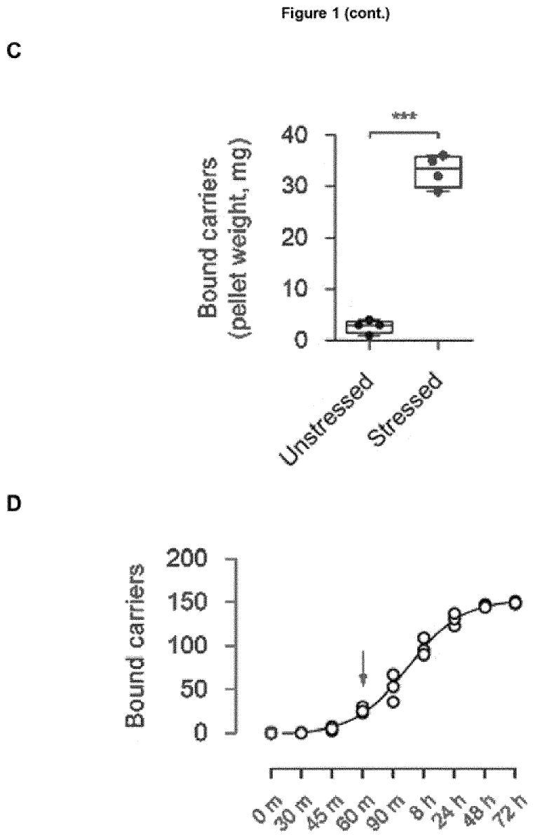

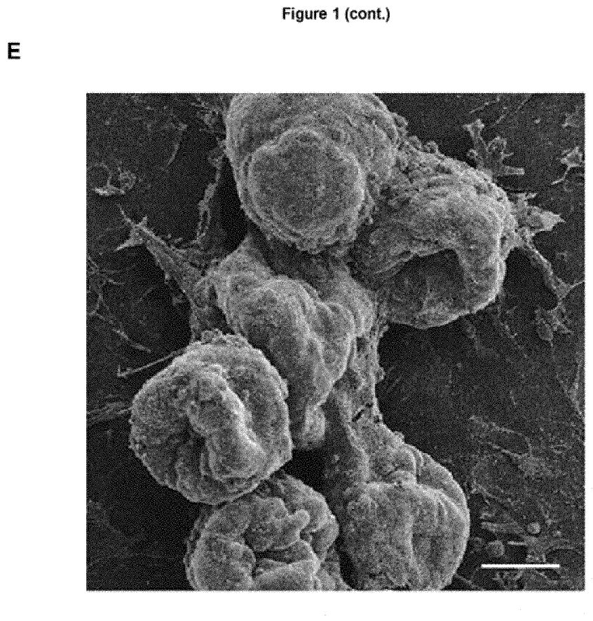

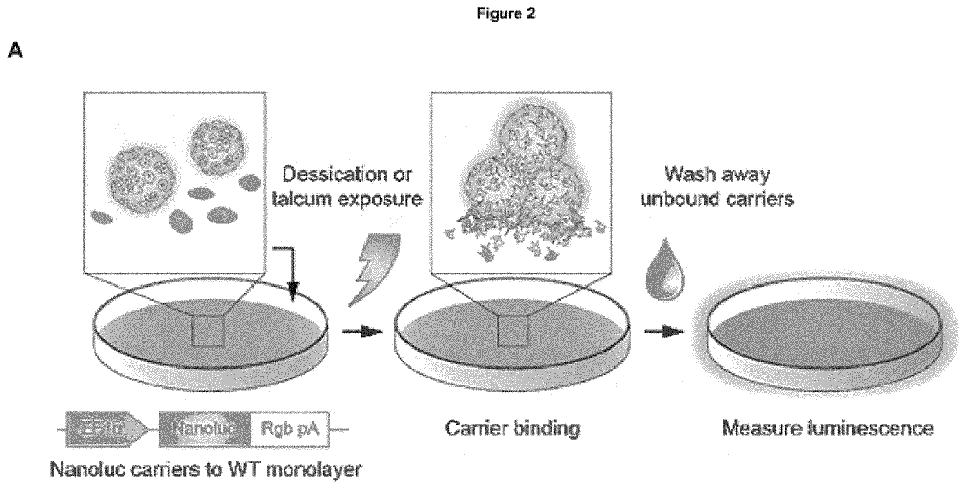

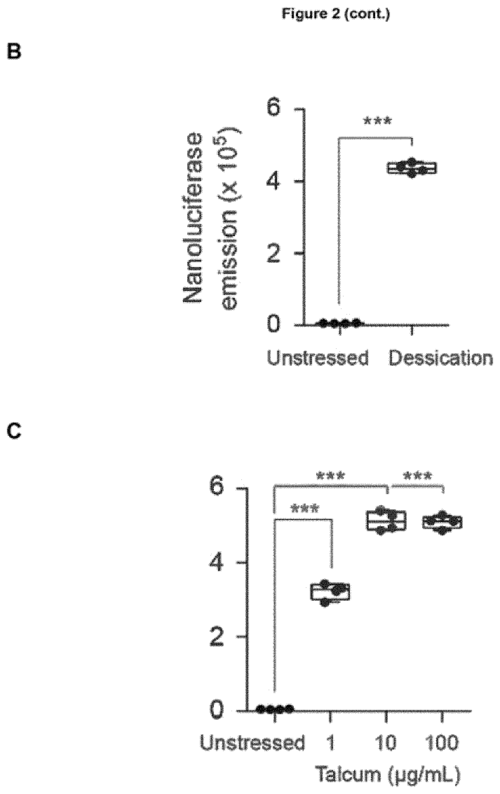

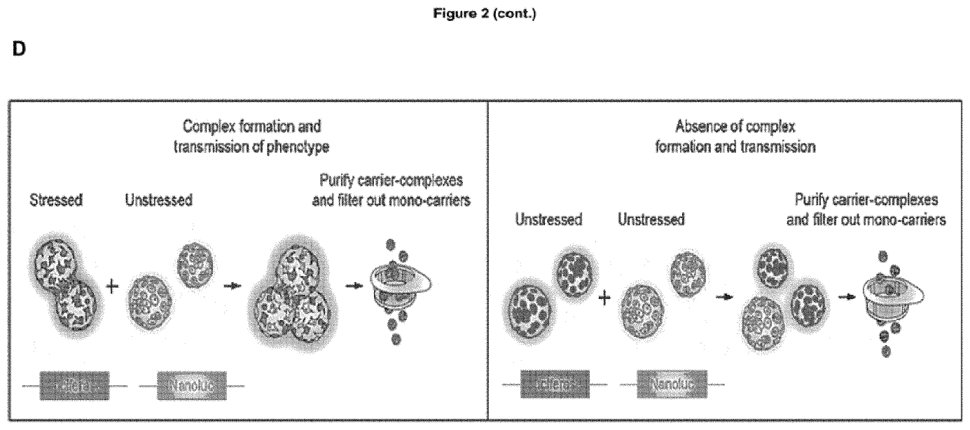

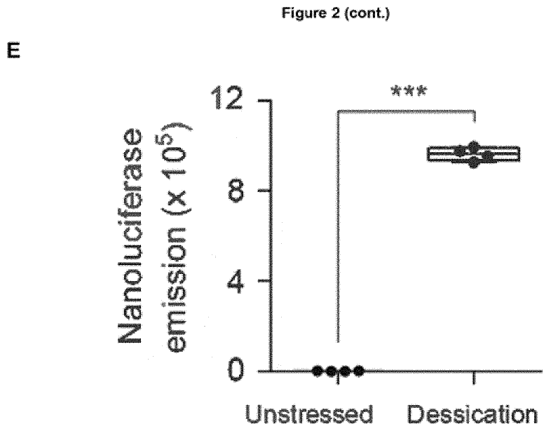

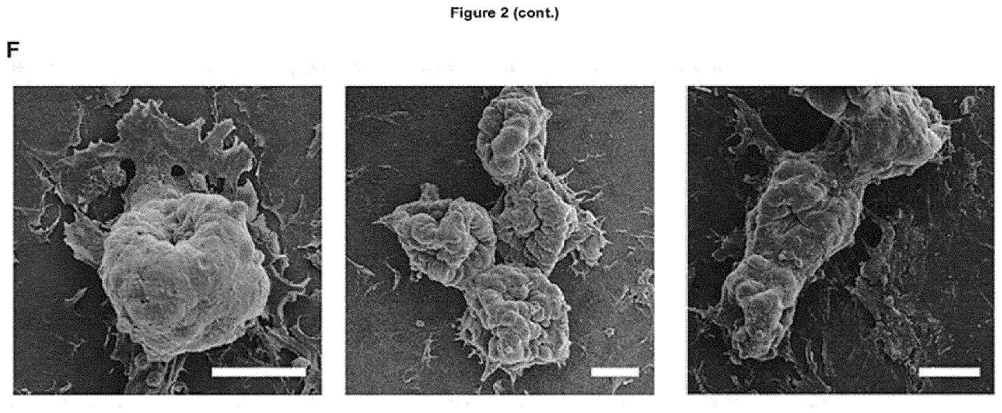

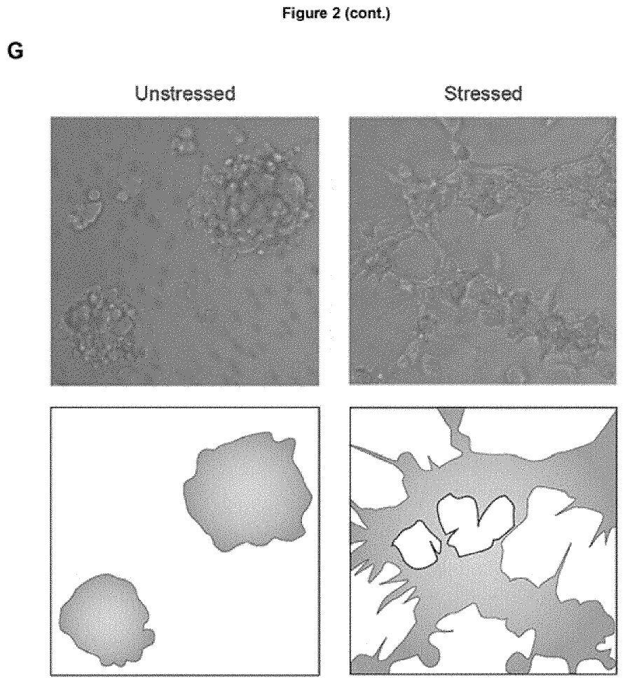

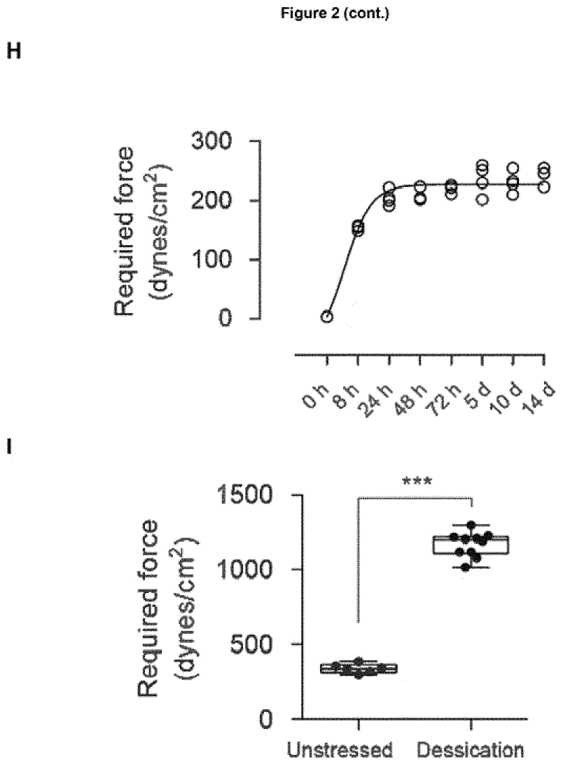

Hence, the activation of said mesothelial cell/stressing said cell may be achieved in vitro by any one of hypoxia, ischemia, inflammation, infection, a chemical stimulus, desiccation, a mechanical trauma (e.g. shearing forces), cold shock, heat shock, osmotic shock, or a foreign body, or any combination thereof. Also provided herein, is the activation of said mesothelial cell being achieved in vitro by hypoxia, ischemia, inflammation, infection, a chemical stimulus, desiccation, a mechanical trauma (e.g. shearing forces), cold shock, heat shock, osmotic shock and a foreign body. In a preferred embodiment of the present invention, a mesothelial cell may be activated/stressed by any one of hypoxia, desiccation, cold shock, heat shock, osmotic shock or a foreign body, or any combination thereof (see FIGS. 1B, 2B and C).

Activating a mesothelial cell by “hypoxic shock (hypoxia)” according to the present invention refers to exposing said cell, which may be seeded on a bead, preferably a microcarrier bead, as mentioned elsewhere herein, to ambient air for at least about 7 minutes, for at least about 10 minutes, or from about 7 to about 30 minutes, or from about 10 to about 23 minutes, preferably applying said cell-covered beads to a cell culture flow hood. In an even more preferred embodiment, activating a mesothelial cell by hypoxia refers to exposing a mesothelial cell to ambient air for about 15 minutes, preferably applying said cell-covered beads to a cell culture flow hood.

Activating a mesothelial cell by “desiccation” (or dehydration) according to the present invention refers to exposing said cell, which may be seeded on a bead, preferably a microcarrier bead, to high fluctuation of air stream (air circulation) for at least about 5 minutes, for at least about 7 minutes, for at least about 10 minutes, or from about 5 to about 30 minutes, or form about 7 to about 23 minutes, or from about 10 to about 15 minutes, after the medium has been removed. In an even more preferred embodiment, activating a mesothelial cell by desiccation refers to exposing a mesothelial cell to high fluctuation of air stream (air circulation) for about 15 minutes, preferably exposing said cell to high fluctuation of air stream (air circulation) under a running workbench for about 15 minutes. In vivo, this may occur naturally during the laparotomy operation (exposure to ambient air and drainage of the peritoneal fluid).

Activating a mesothelial cell by “cold shock” according to the present invention refers to exposing said cell, which may be seeded on a bead, preferably a microcarrier bead, as mentioned elsewhere herein, to at least about −40° C. for at least about 5 minutes, or to at least about −30° C. for at least about 5 minutes, or to a range from about −40° C. to about −10° C. for at least about 5 minutes, or to a range from about −30° C. to about −13° C. for at least about 5 minutes. In an even more preferred prefered embodiment, activating a mesothelial cell by cold shock refers to exposing said cell to about −20° C. for about 10 minutes.

Activating a mesothelial cell by “heat shock” according to the present invention refers to exposing said cell, which may be seeded on a bead, preferably a microcarrier bead, as mentioned elsewhere herein to at least about 21° C. for at least about 5 minutes, or to at least about 28° C. for at least about 5 minutes, or to a range from about 21° C. to about 84° C. for at least about 5 minutes, or to a range from about 28 to about 63° C. for at least about 5 minutes. In an even more preferred embodiment, activating a mesothelial cell by heat shock refers to exposing said cell to about 42° C. for about 10 minutes.

Activating a mesothelial cell by “heat shock” according to the present invention refers to exposing said cell, which may be seeded on a bead, preferably a microcarrier bead, as mentioned elsewhere herein to a foreign body. A foreign body in the context of the present invention may be talcum powder (talcum), suture material, starch glove powder, glucose-containing fluid (e.g. dialysis fluids), surgery patch, mesh or an asbestos particle. Talcum powder (talcum) may be preferred as a foreign body herein.

In a preferred embodiment, activating a mesothelial cell by a foreign body refers to exposing said cell to at least about 5 μg/ml of the foreign body, or to at least about 7 μg/ml of the foreign body, or to a range from about 5 μg/ml to about 20 μg/ml of the foreign body, or to a range from about 7 μg/ml to about 15 μg/ml of the foreign body, preferably talcum. In an even more preferred embodiment, activating a mesothelial cell by a foreign body refers to exposing said cell to about 10 μg/ml of the foreign body, preferably talcum.

Activating a mesothelial cell by “osmotic shock” according to the present invention refers to exposing said cell, which may be seeded on a bead, preferably a microcarrier bead, as mentioned elsewhere herein to a sudden change in the solute concentration surrounding the cells, causing a rapid change in the movement of water across cell membranes also including the osmotic loss of water from the interstitium of a cell. Such an osmotic shock may occur after dialysis resulting in cell damage.

In another embodiment, the heliocyte as being an activated mesothelial cell of the present invention may show a distinct profile on transcriptional-, proteomic-, morphologic and functional level compared to healthy, non-activated mesothelial cells.

In order to identify underlying mechanisms of heliocyte transformation, the present inventors performed differential gene expression analysis and found out several of the most differentially expressed genes allowing these genes to partition into three distinct hierarchies: 1) actin cross-linkers and cytoskeletal modulators, 2) protein traffickers, and 3) calcium regulators.

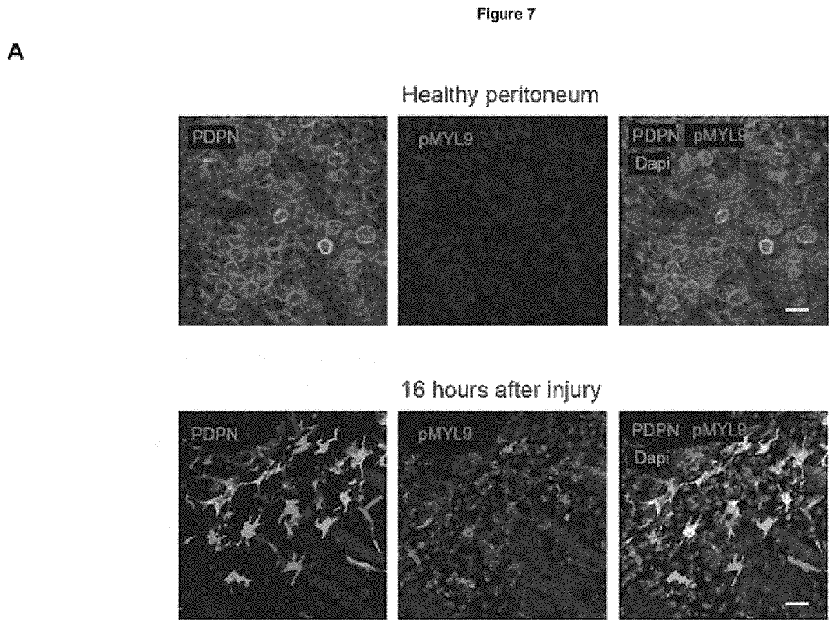

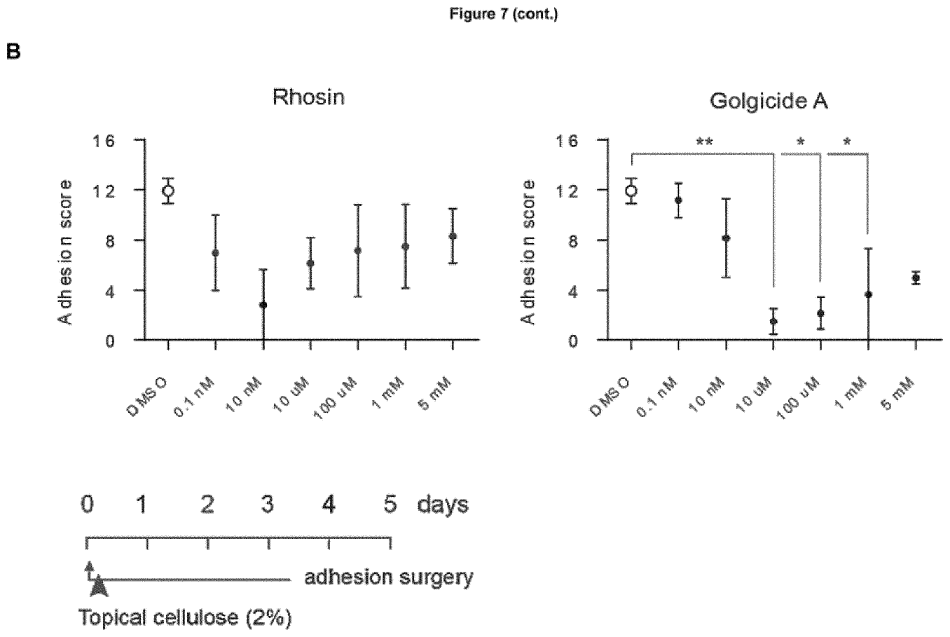

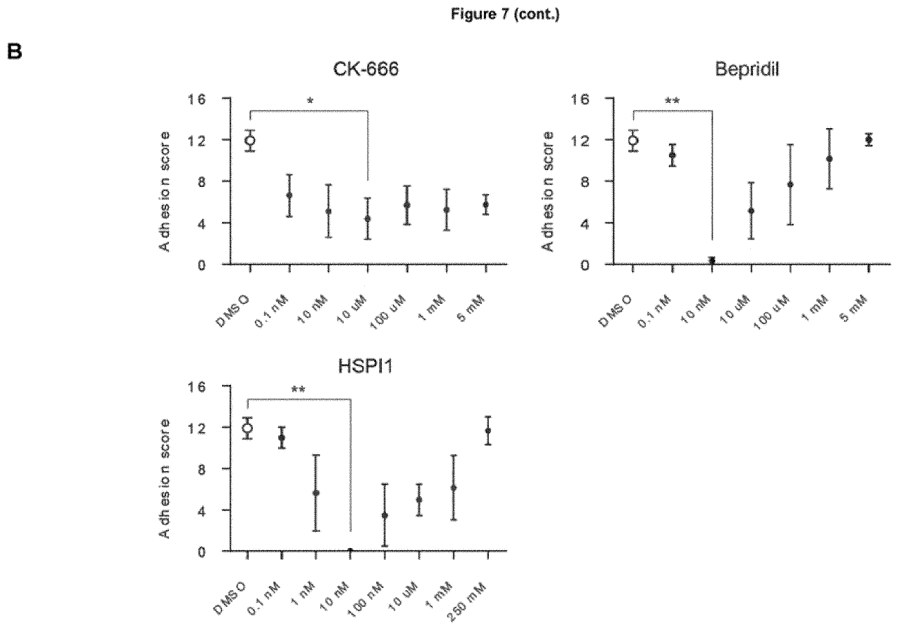

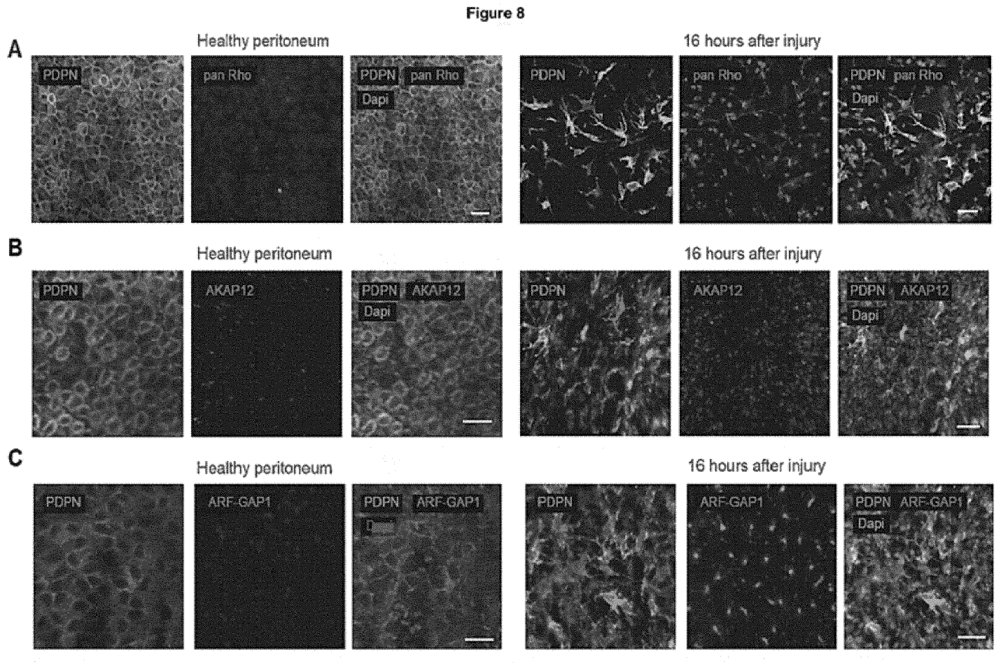

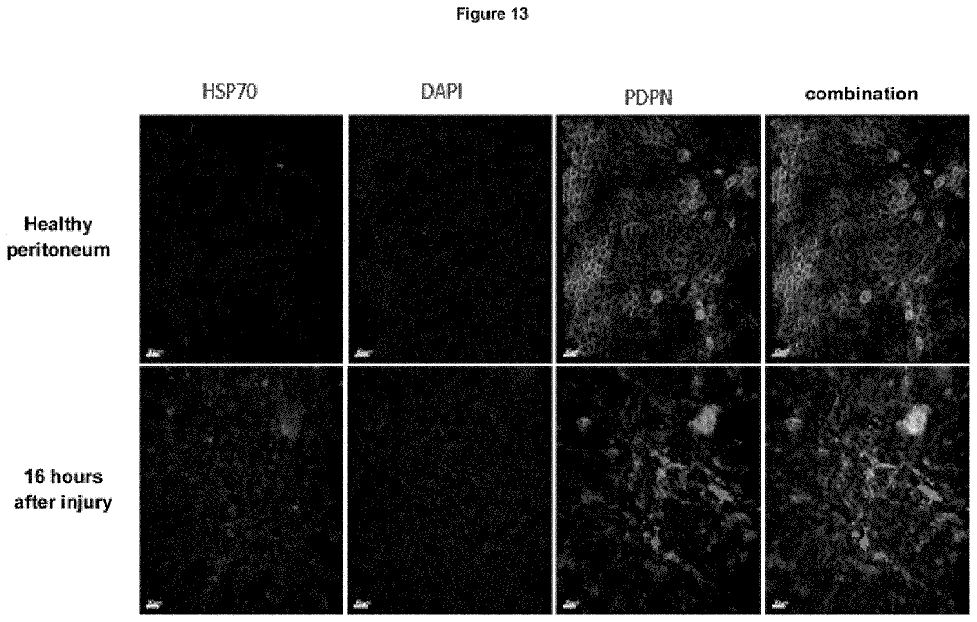

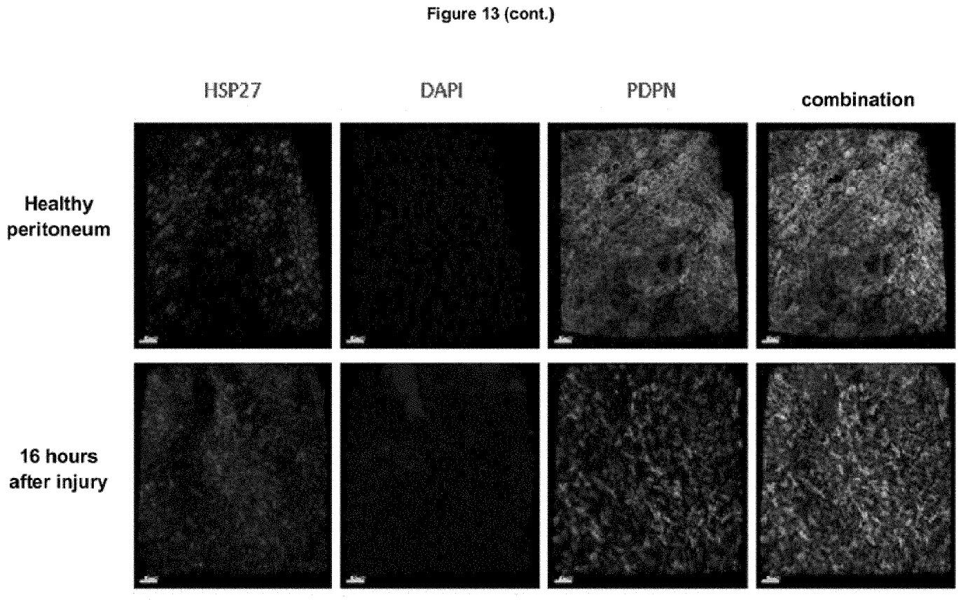

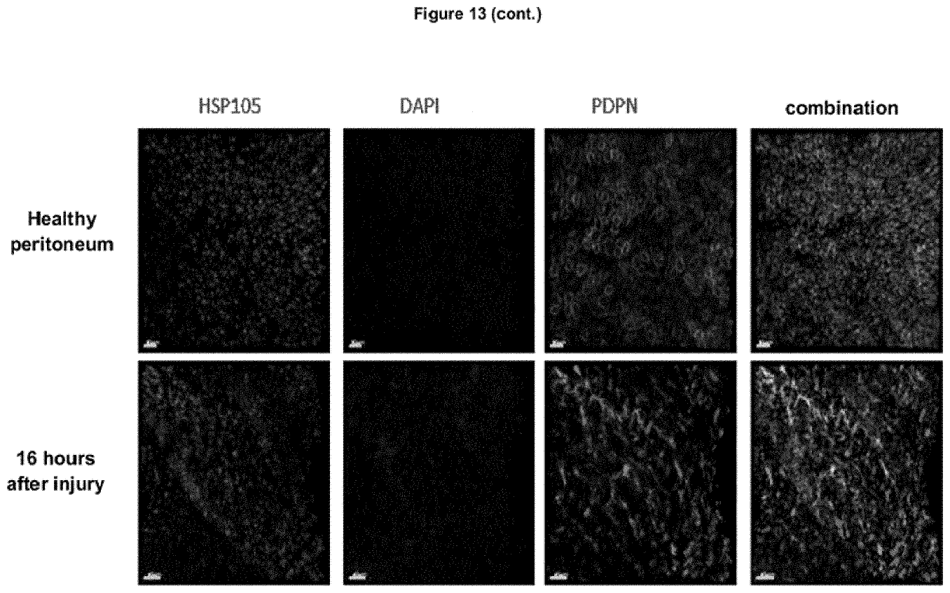

Heliocytes may functionally be identifiable based on the following markers: pan Rho GTPase (also called Rho), which is a family of well-known G proteins that control intracellular actin dynamics and cytoskeletal programming²⁴, ADP-ribosylation factor GTPase-activating protein 1 (also called ARF-GAP1), which is a Golgi-associated enzyme that regulates protein trafficking²³, A-kinase anchor protein 12 (also called AKAP12) being a compartmentalizing protein that localizes at the membrane and is regulated by intracellular calcium²⁶, heat shock protein 70 (HSP70), which interacts with extended peptide segments of proteins as well as partially folded proteins to prevent aggregation, remodel folding pathways, and regulate activity, heat shock protein 27 (HSP27), which acts as a protein chaperone and an antioxidant and plays a role in the inhibition of apoptosis and actin cytoskeletal remodeling, heat shock protein 105 (HSP105), or phosphorylated Myosin 9 light chain (pMLC9), which is a calcium-sensitive regulatory protein that is necessary for cytoskeletal dynamics²⁵. These markers on the protein level may be uniquely expressed on heliocytes of the present invention, and are absent on naive mesothelium (see FIG. 7A and FIG. 8A, B, C and FIG. 13).

The activated mesothelial cell in comparison to a non-activated mesothelial cell may thus be characterized by an increased expression of any one of Rho, ARF-GAP1, AKAP12, HSP70, HSP27, HSP105, or phosphorylated MLC9 on the protein level, or any combination thereof. Thus, any one of the markers mentioned above may be sufficient to accurately identify a heliocyte in the context of the present invention, given the selective nature of these markers and the absence of said markers in a healthy mesothelial cell. The activated mesothelial cell in comparison to a non-activated mesothelial cell may also be characterized by an increased expression of Rho, ARF-GAP1, AKAP12, HSP70, HSP27, HSP105, and phosphorylated MLC9 (pMLC9) on the protein level. Taken together these markers reflect a changed cytoskeletal dynamic of a heliocyte in comparison to a mesothelial cells and/or activated mesothelial cells.

HSP70, HSP27 and HSP105 may act individually but also as a complex. Thus, the activated mesothelial cell in comparison to a non-activated mesothelial cell may also be characterized by increased expression of Rho, ARF-GAP1, AKAP12, the complex of HSP70/HSP27/HSP105 and phosphorylated MLC9 on the protein level. The activated mesothelial cell in comparison to a non-activated mesothelial cell may also be characterized by increased expression of any one of Rho, ARF-GAP1, AKAP12, the complex of HSP70/HSP27/HSP105 or phosphorylated MLC9 on the protein level, or any combination thereof.

The term “expression” has its art-established meaning and defines, in a quantitative sense, the degree of transcription of a given gene and/or translation of a given protein. In the present invention it is understood that expression refers to protein expression. Means and methods for determining expression data on protein level are detailed further below.

The term “on protein level” as used herein refers to determining the expression of a given protein, preferably of the defined proteins, which characterize a heliocyte of the present invention as mentioned above. The term “determine” or “determining” as used throughout the entire description refers to detect or detecting.

Determining the expression data on protein level is preferably effected by antibodies, in particular by antibodies which are specific for the given marker protein defining the heliocyte of the present invention. Antibodies are preferably used for determining the above mentioned protein level.

Antibodies may be labeled, or bound antibodies may in turn be detected by using labeled (secondary) antibodies. Preferred labels are fluorescent, luminescent and radioactive labels. Particularly preferred are fluorescent labels. The latter type of detection scheme is also known in the art as immunofluorescence. A further art-established alternative are enzyme-linked immunosorbent assays (ELISA).

The term “antibody” includes monoclonal antibodies, polyclonal antibodies, single chain antibodies, or fragments thereof that specifically bind said peptide or polypeptide, also including bispecific antibodies, synthetic antibodies, antibody fragments, such as Fab, a F(ab₂)′, Fv or scFv fragments etc., or a chemically modified derivative of any of these. Monoclonal antibodies can be prepared, for example, by the techniques as originally described in Köhler G and Milstein C, Nature 256 495-7 (1975), and Galfré G and Milstein C, Meth. Enzymol. 73 3-46 (1981), which comprise the fusion of mouse myeloma cells to spleen cells derived from immunized mammals with modifications developed by the art. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to an epitope of the peptide or polypeptide of the invention (Schier, Human Antibodies Hybridomas 7 97-105 (1996); Malmborg, J. Immunol. Methods 183 7-13 (1995)). The production of chimeric antibodies is described, for example, in WO89/09622. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735. Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning: A Laboratory Manual, Cold Spring Harbor Laboratory, Cold Spring Harbor, N.Y., 1989.

The term “monoclonal” or “polyclonal antibody” (see Harlow and Lane, (1988), loc. cit.) also relates to derivatives of said antibodies which retain or essentially retain their binding specificity.

The term “scFv fragment” (single-chain Fv fragment) is well understood in the art and preferred due to its small size and the possibility to recombinantly produce such fragments.

Preferably, the antibody, fragment or derivative thereof specifically binds the target protein. The term “specifically binds” in connection with the antibody used in accordance with the present invention means that the antibody etc. does not or essentially does not cross-react with (poly)peptides of similar structures. Cross-reactivity of a panel of antibodies etc. under investigation may be tested, for example, by assessing binding of said panel of antibodies etc. under conventional conditions (see, e.g., Harlow and Lane, (1988), loc. cit.) to the (poly)peptide of interest as well as to a number of more or less (structurally and/or functionally) closely related (poly)peptides. Only those antibodies that bind to the (poly)peptide/protein of interest but do not or do not essentially bind to any of the other (poly)peptides which are preferably expressed by the same tissue as the (poly)peptide of interest, are considered specific for the (poly)peptide/protein of interest.

In a particularly preferred embodiment of the method of the invention, said antibody or antibody binding portion is or is derived from a human antibody or a humanized antibody. The term “humanized antibody” means, in accordance with the present invention, an antibody of non-human origin, where at least one complementarity determining region (CDR) in the variable regions such as the CDR3 and preferably all 6 CDRs have been replaced by CDRs of an antibody of human origin having a desired specificity. Optionally, the non-human constant region(s) of the antibody has/have been replaced by (a) constant region(s) of a human antibody. Methods for the production of humanized antibodies are described in, e.g., EP-A1 0 239 400 and WO90/07861.

In the context of the present invention, the term “increased expression” of said defined heliocyte markers means that the expression of said heliocytes markers is significantly higher compared to the expression of said markers in non-activated mesothelial cells. As already mentioned above, the markers selected from the group consisting of Rho, ARF-GAP1, AKAP12, HSP70, HSP27, HSP105, and phosphorylated MLC9 may not be expressed in a healthy mesothelial cell (FIGS. 8A, B and C). Thus, “an increased expression” of said markers in a heliocyte is determined in comparison to a healthy mesothelial cell not expressing said markers. An “increased expression” of said markers refers to an expression of said markers being at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or even 100% increased/higher compared to the expression of said markers as mentioned above in a non-activated mesothelial cell. In a preferred embodiment, “increased expression” of said defined heliocyte markers refers to an expression of at least about 50%, or being from about 50% to about 100%, or from about 55% to about 100%, or from about 60% to about 100%, or from about 65% to about 100%, or from about 70% to about 100%, or from about 75% to about 100%, or from about 80% to about 100%, or from about 85% to about 100%, or from about 90% to about 100%, or from about 95% to about 100% increased/higher compared to the expression of said markers as mentioned above in a non-activated mesothelial cell.

In particular, with regard to the phosphorylated MLC 9 marker (pMLC9), the term “phosphorylated” refers to having at least about 1%, 2%, 3%, 4%, 5%, 6%, 7%, 8%, 9%, 10%, 15%, 20%, 25%, 30%, 35%, 40%, 45%, 50%, 60%, 65%, 70%, 75%, 80%, 85%, 90%, 95%, or even 100% more phosphorylation on the myosin 9 light chain expressed in a heliocyte in comparison to the phosphorylation of the myosin 9 light chain expressed in a healthy, non-activated mesothelial cell. In a preferred embodiment, the term “phosphorylated” refers to having at least about 50%, or having from about 50% to about 100%, or from about 55% to about 100%, or from about 60% to about 100%, or from about 65% to about 100%, or from about 70% to about 100%, or from about 75% to about 100%, or from about 80% to about 100%, or from about 85% to about 100%, or from about 90% to about 100%, or from about 95% to about 100% more phosphorylation on the myosin 9 light chain expressed in a heliocyte in comparison to the phosphorylation of the myosin 9 light chain expressed in a healthy, non-activated mesothelial cell. The phosphorylated version of MLC9 refers to the activated form of the MLC9 protein. In the present invention the term “(p)Myl9” may also be used interchangeably with the term “(p)MLC9”.

In general, there are several classes of myosins, a superfamily of motor proteins best known for their roles in muscle contraction and in a wide range of other motility processes in eukaryotes. Myosin molecules are composed of a head, neck and tail domain. The head domain binds the filamentous actin, whereas the neck domain acts as a binding site for myosin light chains which are distinct proteins that form part of a macromolecular complex and generally have regulatory functions. The regulation of the phosphorylation of myosin light chain is a central process in the control of smooth muscle cell contraction. The Ca²⁺-calmodulin-activated MLC kinase (MLCK) phosphorylates MLC, whereas its dephosphorylation is catalyzed by the MLC phosphatase (MLCP).

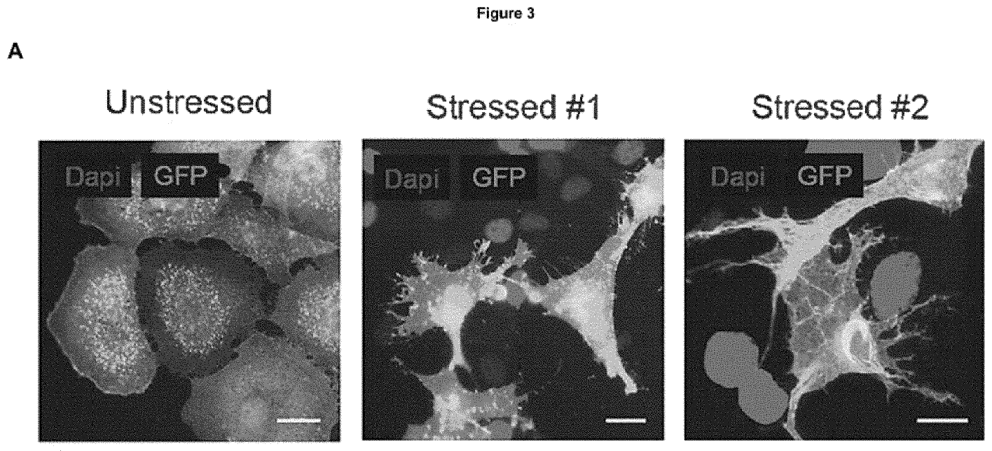

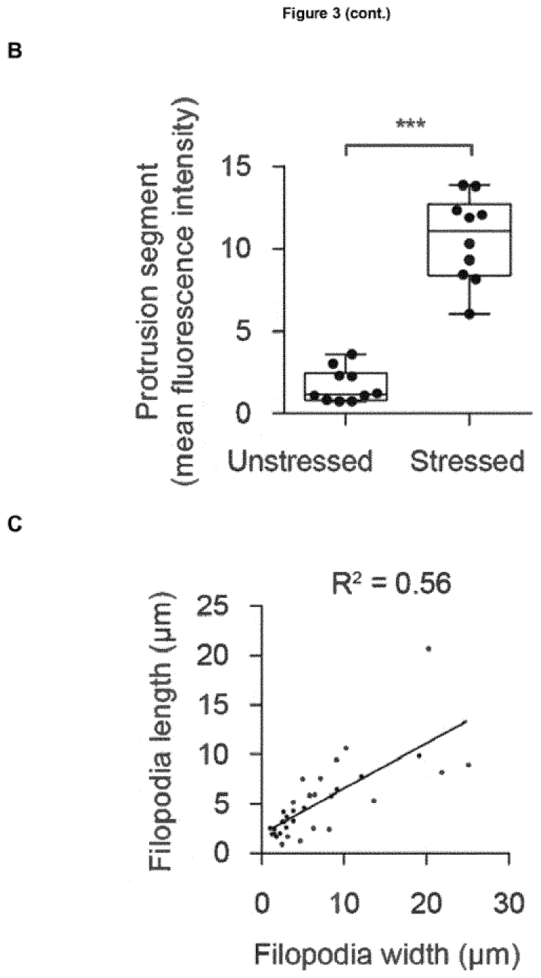

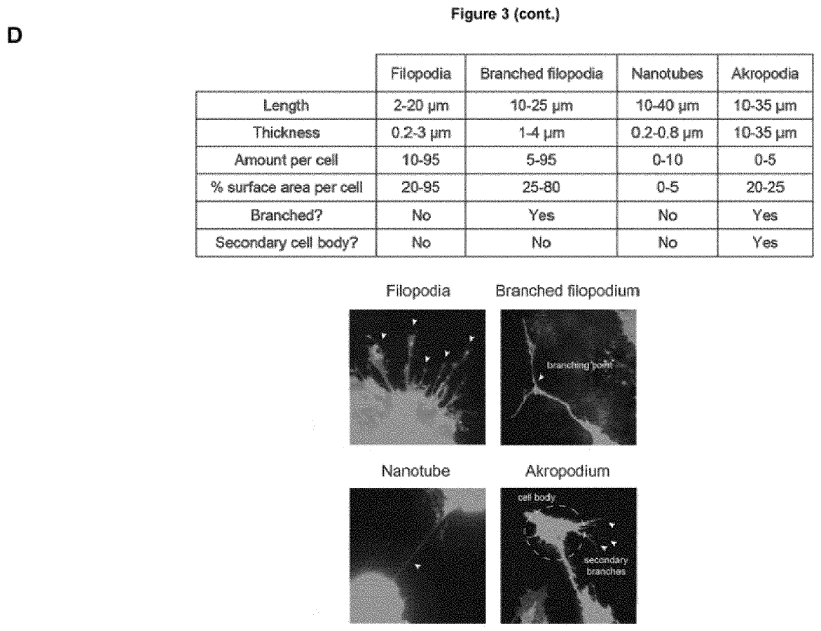

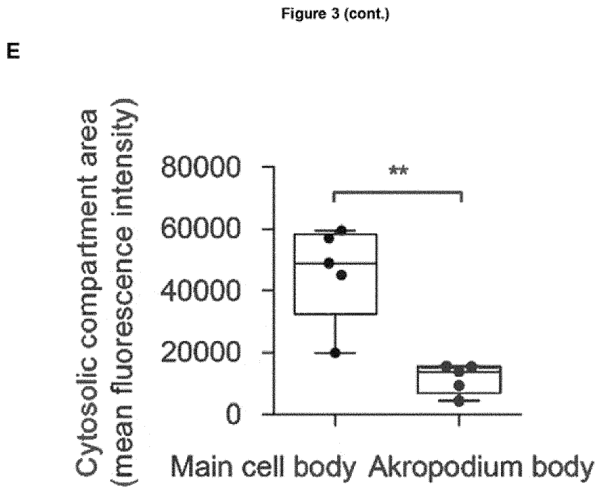

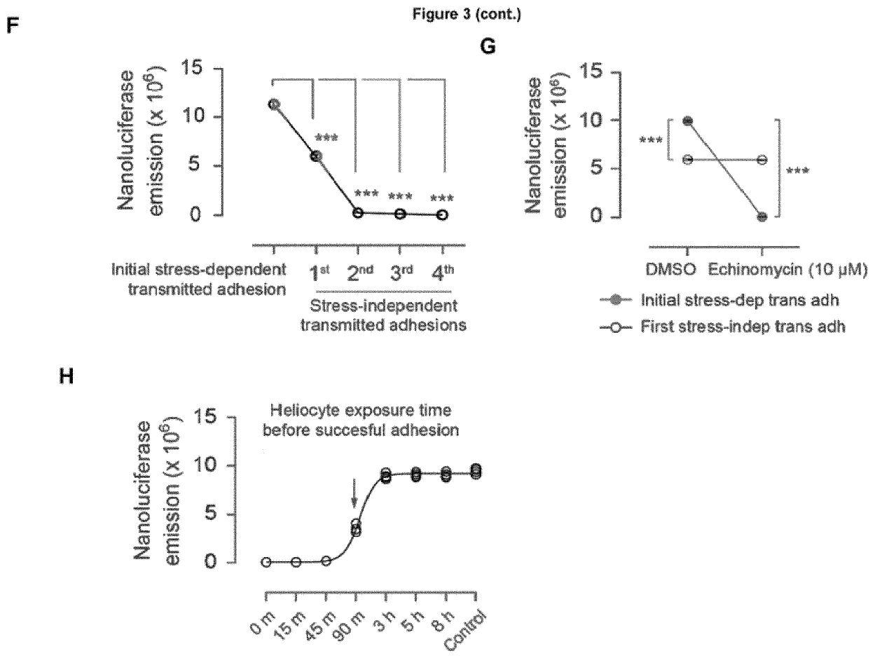

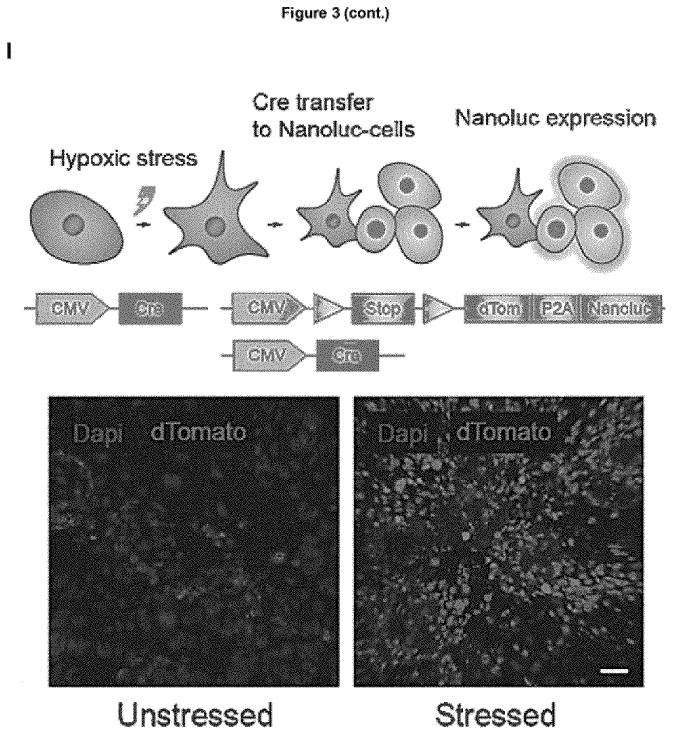

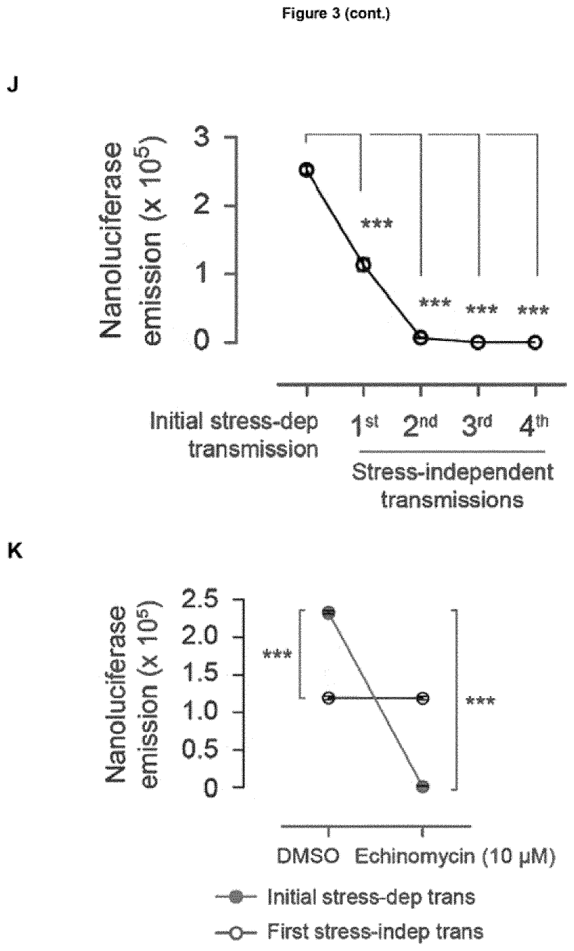

In a further embodiment, a heliocyte of the present invention may be characterized by so called membrane protrusions. Membrane protrusions may also refer to cytoskeletal membrane protrusions. In the present invention, it was found out that after having activated mesothelial cells, preferably being coated on micocarrier beads, via one of the activation stimuli as mentioned above, membrane protrusion growing from the mesothelium could be observed. Therefore, the membrane protrusions of a heliocyte of the present invention may fuse other beads coated with mesothelial cells when applied in the in vitro bead assay of the present invention to form adhesion foci in a matter closely resembling physiological adhesions. The membrane protrusions may intimately also contact adjacent cell surfaces, when beads covered with mesothelial cells were seeded onto a monolayer of mesothelial cells when applied in the in vitro bead assay of the present invention (see FIG. 1E, FIGS. 2F and G, FIG. 3A). This effect may not be observed with non-activated mesothelial cells.

The formation of these membrane protrusions may occur about 0 to about 32 hours, or about 0 to about 24 hours after mesothelial activation. In other words, the formation of these membrane protrusions may occur about 0 to about 32 hours, or about 0 to about 24 hours after a mesothelial cell may be activated by one or more of the activation stimuli mentioned above. In a preferred embodiment, the formation of membrane protrusions may occur about 0 to about 16 hours, such as about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, or about 16 hours after a mesothelial cell may be activated by one or more of the activation stimuli mentioned above.

There may be three different types of membrane protrusions. The akropodia-type, the filopodia-type and the nanotube-type. The filopodia-type may further be divided into a non-branched and a branched filopodia-type (see FIG. 3D).

A heliocyte may thus be characterized by membrane protrusions of the akropodia-type, membrane protrusions of the filopodia-type, wherein membrane protrusions of the filopodia-type may be branched or non-branched and/or by membrane protrusions of the nanotube-type. In other words a heliocyte may be characterized by membrane protrusions of any one of the akropodia-type, the branched filopodia-type, the non-branched filopodia-type, or the nanotube-type, or any combination thereof.

A heliocyte being characterized by membrane protrusions of the akropodia-type may be at least about 5 μm, or at least about 7 μm, or at least about 10 μm in length. In another embodiment membrane protrusions of the akropodia-type may be about 5 to about 70 μm, or about 7 to about 53 μm or about 10 to about 35 μm in length. Preferably, membrane protrusions of the akropodia-type are about 10 to about 35 μm in length. A heliocyte being characterized by membrane protrusions of the akropodia-type may be at least about 5 μm, at least about 7 μm, at least about 10 μm in thickness. In another embodiment, membrane protrusions of the akropodia-type may be about 5 to about 70 μm, or about 7 to about 53 μm or about 10 to about 35 μm in thickness. Preferably, membrane protrusions of the akropodia-type are about 10 to about 35 μm in thickness. A heliocyte being characterized by membrane protrusions of the akropodia-type may in total exhibit/have about 0 to about 10 protrusion(s) per cell, or about 0 to about 8 protrusion(s) per cell, or about 0 to about 5 protrusion(s) per cell. Preferably, a heliocyte being characterized by membrane protrusions of the akropodia-type has about 0 to about 5 protrusion(s) per cell.

Further, membrane protrusions of the akropodia-type may be characterized as having a hand-like morphology, thus being named after the Greek word “akros” (extremity, e.g. hands). Additionally, membrane protrusions of the akropodia-type may be branched and/or exhibit secondary cell bodies. An acropodium (membrane protrusion of the akropodia-type) may essentially refer to a filopodium (membrane protrusion of the filopodia-type) as defined below, however producing/generating a secondary cell body at the tip of its membrane protrusion. In this context, a secondary cell body refers to a cell body which may be formed at the tip of a single large protrusion, thereby distinguishing a secondary cell body from the main heliocyte cell-body which may give rise to a membrane protrusion of the akropodia-type in general. Said secondary cell body may give rise to additional smaller protrusions (extensions) of the filopodia-type. Secondary cell bodies may be highly active, motile and have a long lifetime (>24 h).

The typical surface area of membrane protrusions of the akropodia-type may average at least about 10%, or at least about 13%, or at least about 20%, or from about 10 to about 50%, or from about 13 to about 37%, or from about 20 to about 25% of its main cell body.

A heliocyte being characterized by membrane protrusions of the filopodia-type may be at least about 1 μm, or at least about 1.5 μm, or at least about 2 μm in length. In another embodiment membrane protrusions of the filopodia-type may be about 1 to about 40 μm, or about 1.3 to about 30 μm or about 2 to about 20 μm in length. Preferably, membrane protrusions of the filopodia-type are about 2 to about 20 μm in length. A heliocyte being characterized by membrane protrusions of the filopodia-type may be at least about 0.1 μm, or at least about 0.13 μm, or at least about 0.2 μm in thickness. In another embodiment, membrane protrusions of the filopodia-type may be about 0.1 to about 6 μm, or about 1.3 to about 4.5 μm or about 0.2 to about 3 μm in thickness. Preferably, membrane protrusions of the filopodia-type are about 0.2 to about 3 μm in thickness. A heliocyte being characterized by membrane protrusions of the filopodia-type may in total exhibit/have about 5 to about 190 protrusions per cell, or about 7 to about 143 protrusions per cell, or about 10 to about 95 protrusions per cell. Preferably, a heliocyte being characterized by membrane protrusions of the filopodia-type has about 10 to about 95 protrusions per cell.

Further, membrane protrusions of the filopodia-type may be characterized as not being straight, rather slightly curvy. Additionally, membrane protrusions of the filopodia-type may not be branched and/or exhibit secondary cell bodies.

The typical surface area of membrane protrusions of the filopodia-type may average at least about 10%, or at least about 13%, or at least about 20% of its main cell body. In another embodiment, the typical surface area of membrane protrusions of the filopodia-type may average about 20-95% of its main cell body.

A heliocyte being characterized by membrane protrusions of the branched filopodia-type may be at least about 5 μm, or at least about 7 μm, or at least about 10 μm in length. In another embodiment, membrane protrusions of the branched filopodia-type may be about 5 to about 50 μm, or about 6.5 to about 37.5 μm or about 10 to about 25 μm in length. Preferably, membrane protrusions of the branched filopodia-type are about 10 to about 25 μm in length. A heliocyte being characterized by membrane protrusions of the branched filopodia-type may be at least about 0.5 μm, or at least about 0.7 μm, or at least about 1 μm in thickness. In another embodiment, membrane protrusions of the branched filopodia-type may be about 0.5 to about 8 μm, or about 0.7 to about 6 μm or about 1 to about 4 μm in thickness. Preferably, membrane protrusions of the branched filopodia-type are about 1-4 μm in thickness. A heliocyte being characterized by membrane protrusions of the branched filopodia-type may in total exhibit/have about 3 to about 190 protrusions per cell, or about 3 to about 143 protrusions per cell, or about 5 to about 95 protrusions per cell. Preferably, a heliocyte being characterized by membrane protrusions of the branched filopodia-type has about 5 to about 95 protrusions per cell.

Further, membrane protrusions of the branched filopodia-type may be characterized as not being straight, rather slightly curvy. Additionally, membrane protrusions of the branched filopodia-type may not exhibit secondary cell bodies. In general, branched/non-branched filopodia (membrane protrusion of the filopodia-type) may refer to thin, finger-like structures that are filled with tight parallel bundles of filamentous actin, unlike lamellipodia, which contain sheet-like protrusions that are filled with a branched network of actin. Filopodia may protrude from lamellipodia, and are involved in adhesion, guidance, sensing, and growth. In this context, the term “branched” refers to protrusions which originate not from the mail cell body of the heliocyte, but from protrusions themselves. The term branched may also refer to having sub-branches (protrusions form protrusions) originating from branches (protrusions), which may derive from the main cell body of a heliocyte of the present invention.

The typical surface area of membrane protrusions of the branched filopodia-type may average at least about 12%, or at least about 17%, or at least about 25% of its main cell body. In another embodiment, the typical surface area of membrane protrusions of the branched filopodia-type may average about 25 to about 80% of its main cell body.

A heliocyte being characterized by membrane protrusions of the nanotube-type may be at least about 5 μm, or at least about 7 μm, or at least about 10 μm in length. In another embodiment membrane protrusions of the nanotube-type may be about 5 to about 80 μm, or about 6.5 to about 60 μm or about 10 to about 40 μm in length. Preferably, membrane protrusions of the nanotube-type are about 10 to about 40 μm in length. A heliocyte being characterized by membrane protrusions of the nanotube-type may be at least about 0.1 μm, or at least about 0.13 μm, or at least about 0.2 μm in thickness. In another embodiment, membrane protrusions of the nanotube-type may be about 0.1 to about 1.6 μm, or about 0.13 to about 1.2 μm or about 0.2 to about 0.8 μm in thickness. Preferably, membrane protrusions of the nanotube-type are about 0.2 to about 0.8 μm in thickness. A heliocyte being characterized by membrane protrusions of the nanotube-type may in total exhibit/have about 0 to about 20 protrusion(s) per cell, or about 0 to about 15 protrusion(s) per cell, or about 0 to about 10 protrusion(s) per cell. Preferably, a heliocyte being characterized by membrane protrusions of the nanotube-type has about 0 to about 10 protrusion(s) per cell.

Further, membrane protrusions of the nanotube-type may be characterized as straight and very long, preferably under constant tension. Additionally, membrane protrusions of the nanotube-type may not be branched and/or exhibit secondary cell bodies.

The typical surface area of membrane protrusions of the nanotube-type may average at least about 1%, or at least about 2.5%, or at least about 3%, or from about 0 to about 10%, or from about 0 to about 7.5%, or from about 0 to about 5% of its main cell body.

In a preferred embodiment, membrane protrusions analysis including length, width, total surface area is performed using a collection of machine learning algorithms for segmentation, even more preferably the Advanced weka segmentation Fiji plugin is used.

In another embodiment, an activated mesothelial cell (a heliocyte according to the present invention) being characterized by membrane protrusions of the present invention as mentioned above may be defined by having a more than 2-fold, or even a more than 3-fold increase of the binding force in comparison to a non-activated mesothelial cell (see FIGS. 2H and I). Here, the term binding force refers to the binding capability of the membrane protrusions defined above. This may be compared to the binding capability to non-activated/unstressed healthy mesothelial cells. The suffix “-fold” refers to multiples. “Onefold” means a whole, “twofold” means twice as much, “threefold” means three times as much.

In another embodiment, a heliocyte of the present invention may be characterized by the occurrence of the secretion of (extracellular) vesicle(s), also called (extracellular) vesicle secretion. In this context, the term “extracellular” means outside the cells.

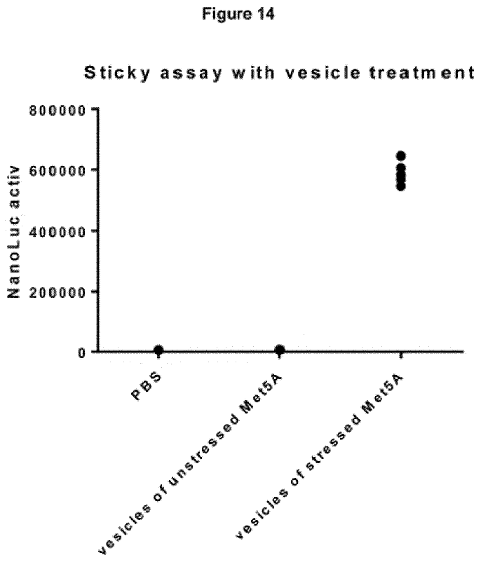

Extracellular vesicles are the microscopic particles secreted by cells in the size of a nano molar unit. In the past, they were regarded as debris secreted from the cells, but they are now considered clinically meaningful. The heliocyte vesicles of the present invention may transport the target proteins of the compound of the present invention. The heliocyte vesicles may also induce adhesion formation without additional activation stimuli when mesothelial cells may be treated with vesicles derived from stressed mesothelial cells (heliocytes) in the in vitro bead assay in comparison to vesicles derived from unstressed mesothelial cells (FIG. 14).

The term “(extracellular) vesicle” in this invention indicates a small sphere surrounded by a membrane originated from cells. This sphere varies greatly depending on the origins of the cells in which it is made or the way it is made. The nucleic acid herein is any of DNA, RNA, microRNA, small interfering RNA(siRNA), small nucleolar RNA(snoRNA) and long non-coding RNA(IncRNA). In a preferred embodiment of the present invention, it is DNA or RNA. As used herein and throughout the entire description, the term “vesicle” refer to any one selected from the group consisting of exosome, ectosome, microvesicle, and apoptotic body.

In a preferred embodiment, a heliocyte may be characterized by the secretion of exosome(s), also called exosome secretion. In particular, exosomes, the spherical follicles released by cells, have lots of information about the mother cell protein and DNA, etc.

Within the context of this invention, the term “exosome” refers to externally released vesicles originating from the endosomic compartment or cells, including besides tumor cells and immune cells, particularly antigen presenting cells, such as dendritic cells, macrophages, mast cells, T lymphocytes, B lymphocytes, the heliocytes of the present invention. More specifically, such vesicles are of endosomal origin and are secreted in the extracellular milieu following fusion of late endosomal multivesicular bodies with the plasma membrane. Methods of producing, purifying or using exosomes for therapeutic purposes or as research tools have been described in WO99/03499, WO00/44389 and WO97/05900, incorporated therein by reference.

In a further embodiment of the present invention, heliocytes may be characterized by developing adhesions (also called adhesion formation).

Adhesion formation in general and in its medical sense refers to conglutination, the process of adhering or uniting of two surfaces or parts. For example, the union of the opposing surfaces of a wound, or opposing surfaces of peritoneum. Also, adhesions, in the plural, can refer to inflammatory bands that connect opposing serous surfaces. Adhesions are pathological bands of fibrous tissue that fuse organ surfaces in response to a myriad of insults. They are a major cause of post-surgical morbidity, bowel obstruction, female infertility, chronic pain, and even death. Adhesions originate from the mesothelium lining organ surfaces.

In the present invention, activation stimuli, which have been defined earlier in the application, may induce massive membrane protrusions in activated mesothelial cells preferably being seeded on beads in the in vitro bead assay of the present invention. Those membrane protrusions may then fuse the beads together which may be covered with mesothelial cells. Those membrane protrusions may then form adhesion foci in a matter that closely resembles physiological adhesions as described above.

Thus, in the context of the present invention the development of adhesions induced by heliocytes may be based on the physical binding of the (cytoskeletal) membrane protrusions selected from any one of the akropodia-type, the (branched) filopodia-type or the nanotube-type, or any combination thereof of one heliocyte to any (healthy) mesothelial cell, a heliocyte may come into contact with. Once a mesothelial cell becomes a heliocyte, said heliocyte may be migratory and may also generate new adhesion foci further away from the initial site where said mesothelial cell became a heliocyte. In particular, the development of adhesions induced by heliocytes may be based on the physical binding of the (cytoskeletal) membrane protrusions selected from any one of the akropodia-type, the (branched) filopodia-type, or the nanotube-type, or any combination thereof to neighboring (healthy) mesothelial cells, most likely at apposing serosal surfaces. Due to the formation of the cytoskeletal protrusions selected from any one of the akropodia-type, the (branched) filopodia-type or the nanotube-type, or any combination thereof, the heliocyte may then be able to transmit its pathological behaviour/its pathogenic phenotype from a heliocyte to a healthy mesothelial cell as described elsewhere herein. After transmission of the pathogenic phenotype, healthy mesothelial cells may also start to form membrane protrusions, thereby binding to other still healthy mesothelial cells, preferably to other neighbouring mesothelial cells for the transmission of their pathogenic phenotype.

The term “adhesions” or “adhesion formation” as used in the present invention thus refers to the binding of surfaces resulting from membrane attachments between opposing activated mesothelial cells. Membrane attachments may be in the form of fusion of membrane protrusions of heliocytes, preferably selected from any one of the akropodia-type, the (branched) filopodia-type or the nanotube-type, or any combination thereof. In this context, the term “fusion of membrane protrusions” refers to a complete fusion or a partial fusion of membrane protrusions. However, it should be noted that fusion in the context of the present invention may not refer to cell fusions in general, implying two cells to completely merge and result with a single cell having two nuclei.

In this context, the term “apposing” as used herein means near to each other or side by side or something in close proximity. The term “serosal surface” as used herein refers to the serous membranes (or serosa), which are two mesothelial cell layers that are separated and secrete serosal fluid.

The development of adhesions/adhesion formation induced by heliocytes may then result in adhesiogenesis.

In this context, the term “adhesiogenesis” refers to the medical or clinical outcome when adhesions (adhesion formation) manifests in pathology.

According to the present invention, there may be several steps demonstrating the early events driving adhesiogenesis:
1) Mesothelial activation may occur (e.g. by one or more of the activation stimulus(i) as mentioned above);
2) After about 0 to about 32 hours, or about 0 to about 24 hours, preferably about 0 to about 16 hours after the activation, generation/formation of heliocytes may start to occur by the formation of cytoskeletal protrusions as defined elsewhere herein. These protrusions allow for physical binding to healthy mesothelial cells;
3) After about 8 to about 48 hours, or about 11 to about 36 hours, preferably about 16 to about 24 hours after the activation, transmission of the pathogenic phenotype from a heliocyte to a healthy mesothelial cell may occur based on the formation of the membrane protrusions of said heliocyte;
4) After about 12 to about 144 hours, or about 16 to about 108 hours, preferably about 24 to about 72 hours after the activation, formation of adhesion may be visible by the fusion of membrane protrusions from one heliocyte to another heliocyte, which may then manifest in the pathology called adhesiogenesis.
5) After about 2 to about 28 days, or about 3 to about 21 days, preferably about 5 to about 14 days after the activation and once heliocytes have completed the adhesion process, they calm down (stop moving) and undergo further commitment to mesenchymal differentiation, essentially becoming fibroblasts. They then start to deposit matrix in the adhesion area to generate a thick macroscopic scar (see FIG. 10).

In a further embodiment, adhesiogenesis being the result of the development of adhesions induced by heliocytes may be inter-organ or intra-organ adhesiogenesis.

In this context, the term “inter-organ” as used herein refers to between any organs of the human body. The term “inter-organ adhesiogenesis” may refer to adhesiogenesis between any organs of the organs of the muscular system, or between any organs of the organs of the digestive system, or between any organs of the organs of the respiratory system, or between any organs of the organs of the urinary system, or between any organs of the organs of the female or male reproductive system, or between any organs of the organs of the endocrine system, or between any organs of the organs of the circulatory system, or between any organs of the organs of the lymphatic system, or between any organs of the organs of the nervous system, or between any organs of the organs of the integumentary system.

The term “inter-organ adhesiogenesis” may also refer to adhesiogenesis between an organ selected from any organ of the muscular system, digestive system, respiratory system, urinary system, female or male reproductive system, endocrine system, circulatory system, lymphatic system, nervous system, or the integumentary system and another organ also selected from any organ of the muscular system, digestive system, respiratory system, urinary system, female or male reproductive system, endocrine system, circulatory system, lymphatic system, nervous system, or the integumentary system.

In this context of the present invention, “intra-organ” refers to within any organ of the human body.

The term “intra-organ adhesiogenesis” as used in the present invention refers to adhesiogenesis within an organ selected from any organ of the muscular system, or within an organ selected from any organ of the digestive system, or within an organ selected from any organ of the respiratory system, or within an organ selected from any organ of the urinary system, or within an organ selected from any organ of the female or male reproductive system, or within an organ selected from any organ of the endocrine system, or within an organ selected from any organ of the circulatory system, or within an organ selected from any organ of the lymphatic system, or within an organ selected from any organ of the nervous system, or within an organ selected from any organ of the integumentary system.

Organs of the muscular system being affected by inter- or intra-organ adhesiogenesis of the present invention may include, but are not limited to human skeleton, joints, ligaments, muscular system, tendons.

Organs of the digestive system being affected by inter- or intra-organ adhesiogenesis of the present invention may include, but are not limited to salivary glands, pharynx, esophagus, stomach, small intestine, large intestine, liver, gallbladder, mesentery, pancreas.

Organs of the respiratory system being affected by inter- or intra-organ adhesiogenesis of the present invention may include, but are not limited to nasal cavity, pharynx, larynx, trachea, bronchi, lung, diaphragm.

Organs of the urinary system being affected by inter- or intra-organ adhesiogenesis of the present invention may include, but are not limited to kidney, bladder, urethra, ureters.

Organs of the female reproductive system being affected by inter- or intra-organ adhesiogenesis of the present invention may include, but are not limited to ovaries uterus, vagina, vulva, clitoris, placenta.

Organs of the male reproductive system being affected by inter- or intra-organ adhesiogenesis of the present invention may include, but are not limited to testes, epididymis, prostate, penis.

Organs of the endocrine system being affected by inter- or intra-organ adhesiogenesis of the present invention may include, but are not limited to pituitary gland, pineal gland, thyroid gland, parathyroid glands, adrenal glands, pancreas.

Organs of the circulatory system being affected by inter- or intra-organ adhesiogenesis may include, but are not limited to heart, arteries, veins, capillaries.

Organs of the lymphatic system being affected by inter- or intra-organ adhesiogenesis of the present invention may include, but are not limited to lymphatic vessel, lymph node, bone marrow, thymus, spleen.

Organs of the nervous system being affected by inter- or intra-organ adhesiogenesis of the present invention may include, but are not limited to nerves.

Organs of the integumentary system being affected by inter- or intra-organ adhesiogenesis of the present invention may include, but are not limited to mammary glands, skin, subcutaneous tissue.

As already mentioned, the present invention discloses a compound for use in a method of reducing the formation of heliocytes. The reduction of the formation of said heliocytes being characterized as mentioned elsewhere herein may be seen as the unifying feature of the present invention. Reducing the formation of heliocytes may be considered as treating the early onset or the initiation phase of adhesiogenesis by treating the causative agent of adhesiogenesis.

In this context, the term “reducing” refers to inhibiting and/or preventing. Thus, the present invention may also disclose a compound for use in a method of inhibiting and/or preventing the formation of heliocytes. Prevention may also include a treatment step or a method of treating.

In one embodiment, the term “formation of heliocytes” refers to the general arrangement/structure/morphology of already existing heliocytes or the existence/occurrence of already existing heliocytes in general which is characterized by the formation of membrane protrusions from any one of the akropodia-type, the (branched) filopodia-type or the nanotube-type and which are essential for the specific and pathogenic phenotype of said heliocytes as mentioned elsewhere herein. In other embodiments, the term “formation of heliocytes” refers to the development/generation of further heliocytes or the development/generation of any heliocytes at all. In another embodiment, the term “formation of heliocytes” refers to a combination of the abovementioned features or any combination thereof. Analyzed differences between mesothelial cells and heliocytes are also illustrated in FIG. 10.

In some embodiments, reducing the formation of heliocytes refers to inhibiting the general arrangement/structure/morphology of already existing heliocytes by inhibiting (blocking) the formation of membrane protrusions as defined elsewhere herein. In this context, the membrane protrusions as defined herein may already be generated/produced by already existing heliocytes. In other words, reducing the formation of heliocytes refers to inhibiting the morphology of already existing heliocytes, which is characterized by its membrane protrusions.

Thus, the present invention may comprise the compound of the present invention being capable of inhibiting the formation of membrane protrusions of the akropodia-type, membrane protrusions of the filopodia-type and/or membrane protrusions of the nanotube-type.

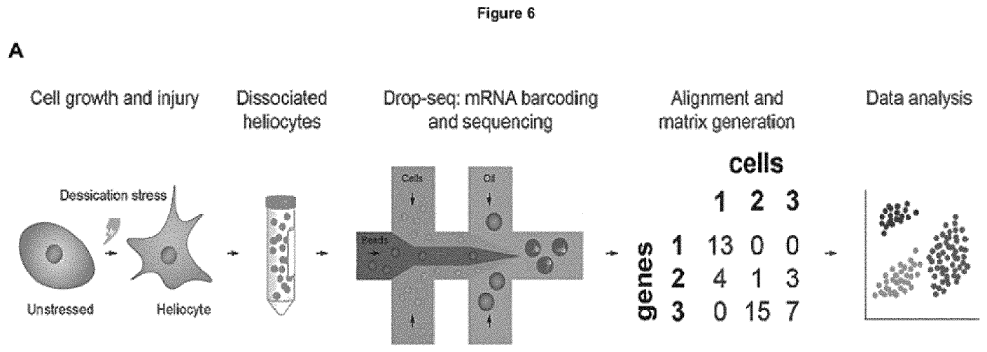

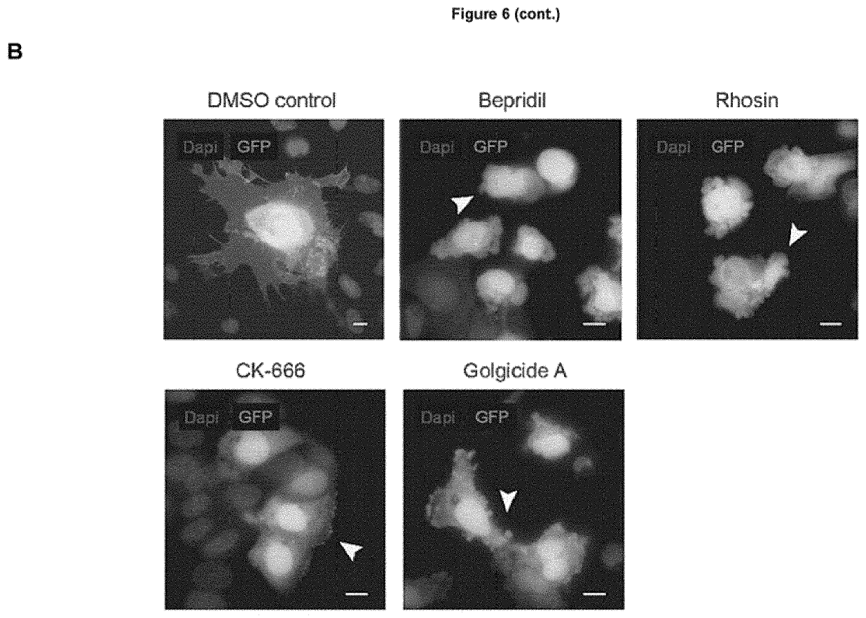

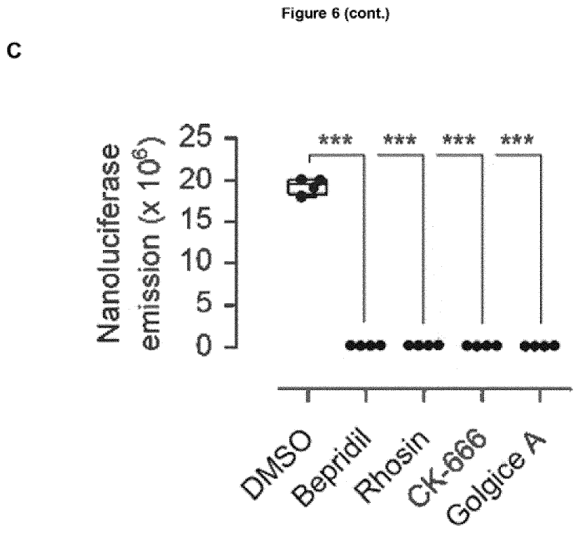

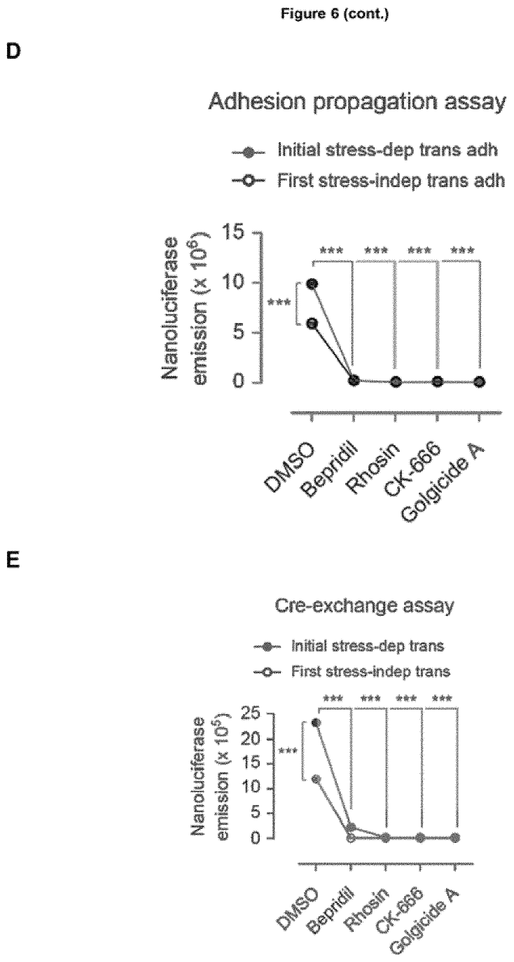

Preferably, the compound Bepridil, Rhosin, CK-666 or Golgicide A is capable of inhibiting (blocking) in vitro the formation of said membrane protrusions defined above in already existing heliocytes, thereby inhibiting the morphology of said heliocytes (see FIG. 6B) in comparison to a control. Even more preferably, at least about 5 μM, or at least about 7 μM, or at least about 10 μM, or from about 5 μM to about 20 μM, or from about 7 μM to about 15 μM, or from about 8 μM to about 12 μM, such as about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM of the compound Bepridil, Rhosin, CK-666 or Golgicide A is capable of inhibiting (blocking) in vitro the formation of said membrane protrusions defined above in already existing heliocytes, thereby inhibiting the morphology of said already existing heliocytes. Most preferably, about 10 μM of the compound Bepridil, Rhosin, CK-666 or Golgicide A is capable of inhibiting (blocking) in vitro the formation of said membrane protrusions defined above in already existing heliocytes, thereby inhibiting the morphology of said heliocytes.

In this context of the present invention and as used throughout the present invention “being capable” or “is capable of” refers to having the biological activity to do something.

In other embodiments, reducing the formation of heliocytes refers to preventing the formation/development/generation of further heliocytes, if heliocytes have already been produced (do exist). In other words, reducing the formation of heliocytes refers to preventing the transmission of the pathogenic phenotype from a heliocyte to a mesothelial cell, thereby preventing the formation of membrane protrusions in healthy mesothelial cells and preventing the healthy mesothelial cells from becoming a further/another/a new heliocyte.

Thus, the present invention may comprise the compound of the present invention being capable of preventing the transmission of mesothelial cell to an activated mesothelial cell e.g. the pathogenic phenotype of a heliocyte and/or back to a healthy mesothelial cell. The term “transmission” as used in the present invention refers to the transfer or passing on or exchange of something from one cell (a heliocyte) to another cell (mesothelial cell), which has not yet received the one to be transmitted. Transmission in the context of the present invention may include the transmission of the pathogenic phenotype of a heliocyte to a healthy mesothelial cell or the transmission of just the cytosolic contents of a heliocyte to a healthy mesothelial cell. The first transmission has been demonstrated by the inventors using the adhesion propagation assay (see methods), where it has been shown that the pathogenic phenotype of a heliocyte may be transmitted, thereby causing a phenotypic change in the healthy mesothelial cell. The second transmission has been demonstrated by the inventors using the Cre-exchange transmission assay (see methods), where it has been shown that transmission of cytosolic contents per se may occur in a heliocyte (that a heliocyte may be able to transmit the cytosolic contents e.g. proteins). In this context, the term “pathogenic phenotype” refers inter alia to the one to be transmitted from one cell (a heliocyte) to another cell (healthy mesothelial cell). In particular, the pathogenic phenotype refers to the capability of forming/developing/generating membrane protrusions selected from the akropodia-type, the (branched) filopodia-type or the nanotube-type or any combination thereof and to the very specific cytosolic contents/blue print within said heliocyte that may instruct those membrane protrusions what to do, e.g. fusing with other heliocytes. Protrusions alone may not be sufficient to generate adhesions. Activated fibroblasts and many other cells also have protrusions, yet they cannot form adhesions. Protrusions are an essential element of the heliocyte program, but the program may also include the specific blue print as mentioned above.

If the formation of further (new) heliocytes may be prevented, this may have a preventive impact or may be seen as precautions.

Preferably, the compound Bepridil, Rhosin, CK-66 or Golgicide A is capable of preventing in vitro the transmission of mesothelial cell to activated mesothelial e.g. to the pathogenic phenotype of a heliocyte and/or back to a healthy mesothelial cell in comparison to a control. The fact that the specific phenotype of heliocytes may be transmitted, has been shown by the present inventors by demonstrating the exchange of cellular contents occurring in general such as the exchange of the Cre recombinase protein being transferred and shuttled from heliocytes into the nucleus of the dTomato-P2A-Nanoluc mesothelial cells (see FIGS. 3I, 6C, D and E). This may underpin the fact that cytosolic contents being essential for the heliocyte being capable of developing membrane protrusions as indicated elsewhere herein may be transferred from heliocytes to healthy mesothelial cells. Even more preferably, at least about 5 μM, or at least about 7 μM, or at least about 10 μM, or from about 5 μM to about 20 μM, or from about 7 μM to about 15 μM, or from about 8 μM to about 12 μM, such as about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM of the compound Bepridil, Rhosin, CK-666 or Golgicide A is capable of preventing in vitro the transmission of a mesothelial cell to an activated mesothelial cell e.g. to the pathogenic phenotype of a heliocyte and/or back to a healthy mesothelial cell. Most preferably, about 10 μM of the compound Bepridil, Rhosin, CK-666 or Golgicide A is capable of preventing said transmission of the mesothelial cell in vitro.

In yet other embodiments, reducing the formation of heliocytes refers to completely destroying the existence/occurrence of already existing heliocytes having already developed/generated membrane protrusions selected from the akropodia-type, the (branched) filopodia-type or the nanotube-type.

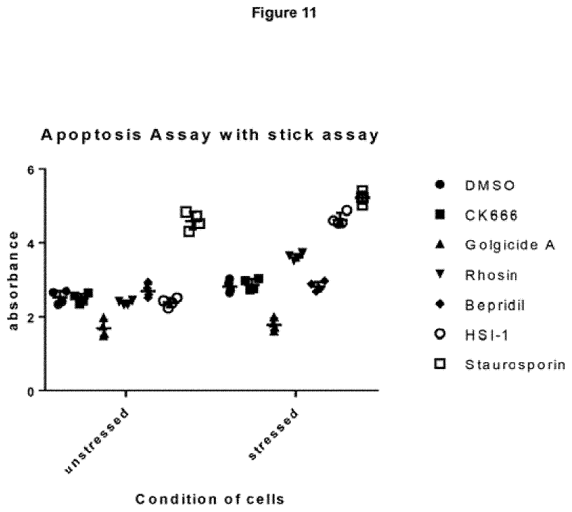

Thus, the present invention may comprise the compound of the present invention being capable of inducing apoptosis in a heliocyte, but not in a mesothelial cell in comparison to a control (see FIG. 11). In this context, the term “apoptosis” refers to a naturally occurring form of programmed cell death. Apoptosis is involved in a variety of normal and pathogenic biological events and can be induced by a number of unrelated stimuli. Currently, apoptosis may be measured by, for example, direct visualization, DNA laddering, flow cytometry (propidium iodide labelling) and measuring the expression of Fas. In the present invention, apoptosis may be measured by specific labeling of nuclear DNA fragmentation which is an indicator for apoptosis. Cells undergoing apoptosis may have an increased amount of fragmented DNA, showing a strong fluorescence signal in the present invention.

In a preferred embodiment, the compound as defined herein is capable of inducing apoptosis in a heliocyte, but not in a mesothelial cell. Even more preferably, at least about 5 μM, or at least about 7 μM, or at least about 10 μM, or from about 5 μM to about 20 μM, or from about 7 μM to about 15 μM, or from about 8 μM to about 12 μM, such as about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM of the compound as defined herein is capable of inducing in vitro apoptosis in a heliocyte, but not in a mesothelial cell. Most preferably, about 10 μM of the compound as defined herein is capable of inducing in vitro apoptosis in a heliocyte, but not in a mesothelial cell. Examples of a working compound concentration of 10 μM for an in vitro assay can be found in FIG. 6. Thus, if not stated otherwise 10 μM is a preferred compound concentration for the compounds defined herein when used in an in vitro assay.

In the present invention it was found that the compound of the present invention may be capable of affecting the biological control of apoptosis, leading to programmed cell death in stressed mesothelial cells (heliocytes), but not in unstressed mesothelial cells. In a preferred embodiment, the compound Rhosin or heat shock protein inhibitor 1 is capable of inducing apoptosis in a heliocyte, but not in a mesothelial cell. Even more preferably, at least about 5 μM, or at least about 7 μM, or at least about 10 μM, or from about 5 μM to about 20 μM, or from about 7 μM to about 15 μM, or from about 8 μM to about 12 μM, such as about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM of the compound Rhosin or heat shock protein inhibitor 1 is capable of inducing in vitro apoptosis in a heliocyte, but not in a mesothelial cell. Most preferably, about 10 μM of the compound Rhosin or heat shock protein inhibitor 1 is capable of inducing in vitro apoptosis in a heliocyte, but not in a mesothelial cell. Apoptosis of heliocytes per se may however not be the immediate mechanism that may prevent adhesions to form. Preventing adhesions to form may preferably be achieved by inhibiting the formation of membrane protrusions as defined elsewhere herein of already existing heliocytes or preventing the formation of protrusions in further heliocytes. Apoptosis of heliocyte may occur later on, being an indirect mechanism.

In yet other embodiments, reducing the formation of heliocytes refers to preventing the formation/development/generation of any future heliocytes. In this context, the term “future” refers to not yet existing or prospective. In other words, reducing the formation of heliocytes refers to the prevention that a heliocyte might develop in the first place, thus having any heliocyte formation at all. The present invention may comprise the compound of the present invention being capable of completely preventing the formation of any future heliocytes, thereby preventing the activation of mesothelial cells.

In yet other embodiments, the term reducing the formation of heliocytes refers to any combination of the definition of said term mentioned in paragraphs [00125], [00130], [00134], or [00137]; or to a combination of paragraphs [00125], [00130], [00134], and [00137].

The present invention may comprise a compound of the present invention being capable of inhibiting the formation of membrane protrusions of the akropodia-type, membrane protrusions of the filopodia-type and/or membrane protrusions of the nanotube-type and being capable of preventing the transmission of a mesothelial cell to an activated mesothelial cell e.g. the pathogenic phenotype of a heliocyte and/or back to a mesothelial cell. In yet another embodiment, the present invention may comprise a compound of the present invention being capable of inhibiting the formation of membrane protrusions of the akropodia-type, membrane protrusions of the filopodia-type and/or membrane protrusions of the nanotube-type and being capable of inducing apoptosis in a heliocyte, but not in a mesothelial cell. In yet another embodiment, the present invention may comprise a compound of the present invention being capable of preventing the transmission of a mesothelial cell to an activated mesothelial cell e.g. the pathogenic phenotype of a heliocyte and/or back to a mesothelial cell and being capable of inducing apoptosis in a heliocyte, but not in a mesothelial cell. In yet another embodiment, the present invention may comprise a compound of the present invention being capable of inhibiting the formation of membrane protrusions of the akropodia-type, membrane protrusions of the filopodia-type and/or membrane protrusions of the nanotube-type, preventing the transmission of the pathogenic phenotype from a heliocyte to a mesothelial cell and inducing apoptosis in a heliocyte, but not in a mesothelial cell.

Said capability of said compound may also comprise determining the capability of a compound to

a) prevent the transmission of a mesothelial cell to the pathogenic phenotype of a heliocyte; and/or
b) induce apoptosis in a heliocyte, but not in a mesothelial cell; and/or
c) prevent the formation of adhesion and/or adhesiogenesis.

Said reduction of the formation of heliocytes is preferably determined in comparison to the same set up normally applied when using the compound of the present invention, however without the compound of the present invention (e.g. using DMSO instead). Said reduction of the formation of heliocytes in comparison to the same set up as mentioned above, but without the compound of the present invention may be at least about 50%, or at least about 55%, or at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99% or even 100%, or from about 50 to about 100%, or from about 60% to about 100%, or from about 70% to about 100%, or from about 80% to about 100%, or from about 90% to about 100%, such as about 50%, 55%, 60%, 65%, 70%, 75%, 80%, 85%, 90%, 95%, 96%, 97%, 98%, 99%, or even 100%. In an even more preferred embodiment, the formation of heliocytes when using the compound of the present invention is completely reduced (e.g. 100% reduced) in comparison to the same set up without the compound of the present invention.

In vivo data demonstrate that even at least about 0.1 nM, or at least about 1 nM, or at least about 2 nM, or at least about 4 nM, or at least about 8 nM, or at least about 10 nM, or at least about 10 μM, or from about 0.1 nM to about 250 mM, or from about 0.1 nM to about 125 mM, or from about 0.1 nM to about 63 mM, or from about 0.1 nM to about 32 mM, or from about 0.1 nM to about 16 mM, or from about 0.1 nM to about 5 mM, or from about 0.1 nM to about 1 mM, or from about 0.5 nM to about 5 mM, or from about 0.5 nM to about 1 mM of the compound of the present invention may be sufficient to reduce the formation of heliocytes. This may be determined by the total adhesion score using morphological features that indicate adhesion development as mentioned in the Example section. The in vivo dosage regimen defined above may also be applied for the pharmaceutical composition of the present invention.

The compound which is capable of preventing the transmission of a mesothelial cell to the pathogenic phenotype of a heliocyte and/or capable of inducing apoptosis in a heliocyte, but not in a mesothelial cell, said capability of said compound is determined by the in vitro bead assay for analyzing heliocytes and/or the formation of adhesions. The present invention further discloses that the capability of said compound being mentioned above may be determined by an in vitro bead assay. Thus, by using the in vitro bead assay of the present invention, where the compound of the present invention is tested therein, the capability of said compound is determined.

An in vitro bead assay as used in the present invention may comprise the steps of
1) seeding mesothelial cells onto a coated dish and letting said cells grow to a monolayer;
2) coating carrier beads with mesothelial cells;
3) eluting said cell-coated beads of step 2) from a culture dish;
4) activating said cells coated on said carrier beads;
5) contacting said activated cell-coated carrier beads with the compound of the present invention;
6) seeding said activated cell-coated carrier beads onto the monolayer of step 1);
7) determining the effect of the compound of the present invention on the activated mesothelial cells coated onto the carriers being seeded onto the monolayer of non-activated mesothelial cells. Said in vitro bead assay is also further illustrated in FIG. 1 and FIG. 2D.

In a preferred aspect of the present invention the in vitro bead assay for analyzing heliocytes and/or the formation of adhesions, comprises the steps of

a) seeding mesothelial cells onto a coated dish and letting said cells grow to a monolayer;
b) coating carrier beads with mesothelial cells;
c) activating said cells coated on said carrier beads of step b) with a stimulus,
d) seeding said activated cells coated on said carrier beads of step b) and c) onto the monolayer of step a); and analyzing the activated mesothelial cells on said carrier beads, or analyzing the activated mesothelial cells eluted from said carrier beads.

Said in vitro bead assay for analyzing heliocytes and/or the formation of adhesions may comprise mesothelial cells which may preferably be Met-5A positive, before being seeded in step a) and/or coated in step b). Additionally, the in vitro bead assay for analyzing heliocytes and/or the formation of adhesions the activated cells on said carrier beads of step c) and/or step d) may be capable of fusing the cell-coated beads together and optionally may be selected by size. It is envisaged by the present invention that the in vitro bead assay for analyzing heliocytes and/or the formation of adhesions, the stimulus of step c) is selected from the group consisting of hypoxia, ischemia, inflammation, infection, a chemical stimulus, desiccation, a mechanical trauma, cold shock, heat shock, osmotic shock, or a foreign body. Preferably, said in vitro bead assay, further comprises

i) contacting said activated cells coated on said carrier beads after step c) with a compound; and
ii) determining the effect of the compound on the activated mesothelial cells and/or formation of adhesions after step d).

In another aspect the present invention relates to an in vitro method for determining the formation of heliocytes, wherein the method comprises obtaining a sample comprising mesothelial cells from a subject, preparing the sample according to the in vitro bead assay for analyzing heliocytes and/or the formation of adhesions and determining the formation of heliocytes in said in vitro bead assay. In yet another aspect the present invention relates to an in vitro method for treating heliocytes and/or adhesions formed by heliocytes, wherein the method comprises obtaining a sample comprising mesothelial cells from a subject, preparing the sample according to the in vitro bead assay for analyzing heliocytes and/or the formation of adhesions and treating the heliocytes and/or adhesions formed by heliocytes by contacting said heliocytes with a compound according to said in vitro bead assay.

According to step 1) and a) of the in vitro bead assay and as used throughout the entire description, when the term “seeding cells” is used, it refers to sowing said cells or plating said cells onto a culture dish, which is coated with a certain substance. Preferably, the dish being used in step 1) and a) is coated with gelatin. In some embodiments, step 1) and a) may be an optional step, thus starting with step 2) and b) as the first step of the in vitro bead assay.

The term “monolayer” as used herein and throughout the description refers to a single, closely packed layer of cells. In some cases it may refer to as a self-assembled monolayer. Monolayers may be well suited for microscopic inspection of growing cells.

In preferred embodiments, the mesothelial cells of said monolayer in step a) may express a reporter construct being recombinase enzyme-dependent. In an even more preferred embodiment, said mesothelial cells of said monolayer in step 1) and a) may express a Cre recombinase-dependent nanoluciferase reporter construct.

According to step 2) and b) of the in vitro bead assay in this context and as used throughout the description, the term “coating” refers to covering. In particular, coating said carrier beads refers to covering/cover said carrier beads. This may be achieved by seeding said mesothelial cells together with said carrier beads. Preferably, mesothelial cells are seeded together with said carrier beads in a ratio of 500:1. This may allow the cells to adhere to the carrier beads. Also, step 2) and b) of the in vitro bead assay may be performed before step 1) and a) or simultaneously.

In a preferred embodiment, said mesothelial cells being coated onto said carrier beads may express a fluorescent protein. In this context, a fluorescent protein may be selected from the group consisting of GFP, mVenus, YFP, dRFP, dTomato, mKate and zsGreen and their corresponding derivates.

In another preferred embodiment, said mesothelial cells being coated onto said carrier beads may express a recombinase enzyme. In this context, a recombinase enzyme may be selected from the group consisting of a Cre recombinase, Hin recombinase, Tre recombinase and FLP recombinase. Preferably, said mesothelial cells being coated onto said carrier beads may express a Cre recombinase enzyme.

In yet another preferred embodiment, said mesothelial cells being coated onto said carrier beads may express a luciferase enzyme, preferably a nanoluciferase.

Also preferred herein, is that according to step 2) and b) microcarriers beads may be used. Microcarrier beads being used in step 2) and b) are more common and known to the person skilled in the art. Some are rigid (such as CYTODEX carrier beads) and some are porous (such as CULTISPHER carrier beads). In an even more preferred embodiment, CYTODEX 3 microcarrier beads are used.

According to step 3) of the in vitro bead assay, elution may be performed by using a filter. This may enable the cell-covered beads to become filtered from the culture dish being used for the mesothelial cells to adhere and grow onto the carrier beads.

According to step 4) and c) of the in vitro bead assay, activating said mesothelial cells may be performed by applying any one of the activation stimuli of hypoxia, ischemia, inflammation, infection, a chemical stimulus, desiccation, a mechanical trauma (e.g. shearing forces), cold shock, heat shock, osmotic shock a foreign body, or any combination thereof. Preferably, activation of said mesothelial cells may be performed by hypoxia, desiccation, cold shock, heat shock, osmotic shock or a foreign body as mentioned elsewhere herein or any combination thereof.

After activation of said cells coated onto carrier beads, said cells may be contacted with the compound of the present invention according to step 5) and i). It may also be disclosed herein, that the step of contacting said cell-coated carrier beads with the compound of the present invention may be performed before the activation step. In a preferred embodiment, contact with the compound of the present invention may be for at least about 10 minutes, or for at least about 15 min, or for at least about 20 minutes, or for at least about 30 minutes, or from about 10 to about 90 minutes, or from about 15 to about 60 minutes, or from about 20 to about 45 minutes, such as about 10, 15, 20, 25, 30, 35, 40, 45, 50, 55, 60, 65, 70, 75, 80, 85, 90 minutes. In an even more preferred embodiment, contact with the compound of the present invention may be for about 30 minutes. The term “to treat” or treatment” may also be used interchangeably with the term “to contact” or “contact”.

According to step 6) and d) of the in vitro bead assay, once a confluent monolayer of mesothelial cells may be established, said cell-coated beads, where the cells have already been activated in step 4) and c) and pretreated in step 5) and i), may then be seeded onto said unstressed monolayer of step 1) and a). In this context, the term “unstressed monolayer” means that the mesothelial cells of the monolayer have not been activated/stressed by any means as mentioned elsewhere herein. The term “monolayer of non-activated mesothelial cells” may also be used interchangeably with the term “unstressed monolayer”. The term “pretreated” as used herein means that the cell-coated beads/cells coated onto beads have already been contacted/treated with the compound of the present invention.

In some embodiments, where step 1) and a) is optional, also step 6) and d) is considered optionally. If step 1) and a) and 6) are optional, the in vitro bead assay of the present invention may be based on a carrier to carrier set up, whereas if step 1) and a) and 6) and d) are not considered as optional, the in vitro bead assay of the present invention may be based on a carrier to monolayer step up.

In general, without applying the compound of the present invention, once said carrier beads may be coated with said mesothelial cells and said cells having been activated, said activated mesothelial cells (also called heliocytes) may fuse with other activated mesothelial cells from other carrier beads by generating membrane protrusions as indicated elsewhere herein in a carrier to carrier set up. In this set up, the beads may thereby cluster together, preferably forming aggregates (FIG. 1A). This may then be observed by detecting fluorescence of said activated mesothelial cells, preferably by stably expressing a fluorescent protein, such as GFP. Said carrier to carrier cluster may be enriched via size exclusion with a filter and the formation may be determined visually, enzymatically or chemically.

In general, without applying the compound of the present invention, once said carrier beads coated with said mesothelial cells may have been seeded onto said monolayer in a carrier to monolayer set up, said already activated mesothelial cells (also called heliocytes) may start to adhere tightly to the monolayer by generating membrane protrusions as indicated elsewhere herein (FIG. 2A). This may then be observed by detecting fluorescence or luminescence or any other way to detect the mesothelial cells/carriers coated with said mesothelial cells of said activated mesothelial cells, preferably by stably expressing a fluorescent protein, such as GFP.

Further, without applying the compound of the present invention, once said carrier beads coated with said mesothelial cells may have been seeded onto said monolayer, the recombinase enzyme, preferably the Cre recombinase enzyme, or the luciferase enzyme, preferably the nanoluciferase enzyme, being expressed by the mesothelial cells coated onto said beads, may be transferred to and shuttled into the nucleus of the cells of the monolayer, thereby transmitting the pathogenic phenotype from a heliocyte (activated mesothelial cell) to a healthy mesothelial cell. Only in the case that the recombinase enzyme may be transferred and shuttled into the nucleus of the cells of the monolayer, the monolayer of mesothelial cells may stably express the construct being recombinase enzyme-dependent. In both cases, bioluminesces, preferably nanoluciferase luminesce may be measured being known to the person skilled in the art, thereby confirming the transmission of the pathogenic phenotype from a heliocyte to a mesothelial cell.

According to step 7) and ii) of the in vitro bead assay as used therein, the term “determining the effect of the compound of the present invention” refers to detecting/investigating the capability of said compound of

I) inhibiting the formation of membrane protrusions of the akropodia-type, membrane protrusions of the filopodia-type and/or membrane protrusions of the nanotube-type of said mesothelial cells being activated as indicated elsewhere herein (FIG. 6B);
II) preventing the transmission of the pathogenic phenotype from a heliocyte to a mesothelial cell (FIGS. 6D and E); or
III) inducing apoptosis in a heliocyte, but not in a mesothelial cell (FIG. 11).

If determining the capability of said compound of inhibiting the formation of membrane protrusions in said in vitro bead assay, the formation of said protrusions (or the arrangement of already existing membrane protrusions) of already activated mesothelial cells coated onto the carrier beads may be inhibited/blocked. Preferably, this may be determined by detecting the fluorescence of said activated mesothelial cells (heliocytes) stably expressing a fluorescent protein. This may emphasize that the compound of the present invention may be capable of inhibiting the morphology of already existing heliocytes, which is characterized by its membrane protrusions.

If determining the capability of said compound of preventing the transmission of the pathogenic phenotype from a heliocyte to a healthy mesothelial cell in said in vitro bead assay, bioluminescence may be completely reduced in the monolayer of mesothelial cells (no detectable luciferase (preferably nanoluciferase) emission any more). This may be due to the fact that the recombinase enzyme or the luciferase enzyme may not be transferred or shuttled to the nucleus of the cells of the monolayer. Again, this may emphasize that the compound of the present invention may be capable of preventing the transmission of a mesothelial cell to an activated mesothelial cell e.g. the pathogenic phenotype of a heliocyte and/or back to a healthy mesothelial cell as indicated elsewhere herein.

If determining the capability of said compound of inducing apoptosis in a heliocyte, but not in a mesothelial cell, an increase amount of fragmented DNA as an indicator for apoptosis may be detected in activated mesothelial cells (heliocytes) but not in non-activated mesothelial cells. Preferably, this may be determined by showing a strong fluorescence signal. Again, this may emphasize that the compound of the present invention may be capable of inducing apoptosis in a heliocyte, but not in a healthy, non-activated mesothelial cell.

Preferably, for each step of the in vitro bead assay, no matter if the mesothelial cells may be coated onto carrier beads or if the mesothelial cells may be allowed to grow to a monolayer, mammalian mesothelial cells are used. Even more preferably, for each step of the in vitro bead assay, no matter if the mesothelial cells may be coated onto carrier beads or if the mesothelial cells may be allowed to grow to a monolayer, human mesothelial cells are used. Most preferably, for each step of the in vitro bead assay, no matter if the mesothelial cells may be coated onto carrier beads or if the mesothelial cells may be allowed to grow to a monolayer, Met-5A cells are used.

As mentioned elsewhere herein, a heliocyte may be characterized by an increased expression of certain markers such as Rho, ARF-GAP1, AKAP12, HSP70, HSP27, HSP105 and/or phosphorylated myosin 9 light chain, which may be clustered to different pathways depending on their clinical relevance:

1) cytoskeletal remodeling
2) protein trafficking
3) calcium signaling
4) heat shock protein signaling.

The compound being used in the present invention may be defined as a compound being able to prevent or inhibit mesothelium to heliocyte transformation (in other words being able to block heliocyte formation) by blocking cytoskeletal remodeling, blocking protein trafficking, blocking calcium signaling, and/or blocking heat shock protein signaling or any combination thereof. The term “heat shock protein signaling” may also refer to “heat shock transcription factor signaling”, heat shock transcription factors modulating a broader spectrum of effectors in comparison to heat shock proteins. It may also be comprised herein, that the compound of the present invention may be able to block cytoskeletal remodeling, block protein trafficking, and/or block calcium signaling or any combination thereof.

In one embodiment the compound being used in the present invention may be defined as a compound being able to block cytoskeletal remodeling. In another embodiment, the compound being used in the present invention may be defined as a compound being able to block protein trafficking. In yet another embodiment, the compound being used in the present invention may be defined as a compound being able to block calcium signaling. In yet another embodiment, the compound being used in the present invention may be defined as a compound being able to block heat shock protein signaling. In a preferred embodiment the compound of the present invention blocks cytoskeletal remodeling.

The present invention may also comprise a compound being able to block more than one of the defined pathways above. The compound may block cytoskeletal remodeling and protein trafficking, or the compound may block cytoskeletal remodeling and calcium signaling. It may also be comprised in the present invention that the compound of the present invention may be able to block protein trafficking and calcium signaling. It may further be comprised that the compound of the present invention may be able to block cytoskeletal remodeling, protein trafficking and calcium signaling.

In this context, the term “block” or “blocking” refers to “inhibit” or “inhibiting”, meaning that the compound of the present invention may be able to target and then inhibit the abovementioned pathways of cytoskeletal remodeling, protein trafficking and/or calcium signaling or a combination thereof. Thus, the compound of the present invention may be seen as a modulator of said pathways, preferably the compound of the present invention is an inhibitor. The compound of the present invention being an inhibitor may target cytoskeletal effectors, protein traffickers, calcium regulators, and/or heat shock proteins (or heat shock transcription factors) or a combination thereof.

The term “cytoskeleton remodeling” may refer to the biochemical process allowing for the dynamic alterations of cellular organization. The cytoskeleton itself may refer to a complex network of interlinking filaments including but not being limited to microfilaments, microtubules, intermediate filaments any myofilaments, and all kinds of tubules as known to the person skilled in the art extending throughout the cytoplasm. This process may occur in mesothelial cells and may also maintain in activated mesothelial cells.

“Protein trafficking” as used herein may refer to secretory protein translocation, folding, and/or assembly in the endoplasmatic reticulum, whereas calcium signaling may refer to a cellular signaling mediated by calcium. Once calcium ions enter the cytoplasm, allosteric regulatory effects on many enzymes and proteins may occur. This may be achieved by using calcium acting in signal transduction resulting from an activation of said ion channels or as a second messenger caused by indirect signal transduction pathways such as G protein-coupled receptors.

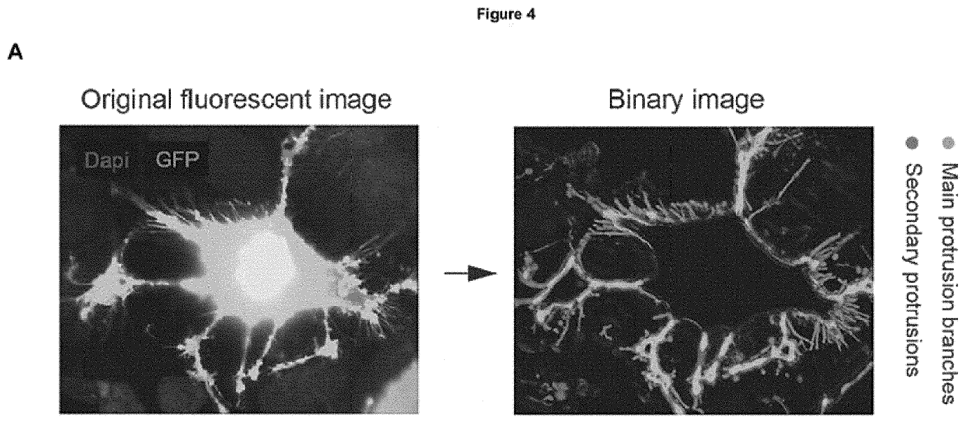

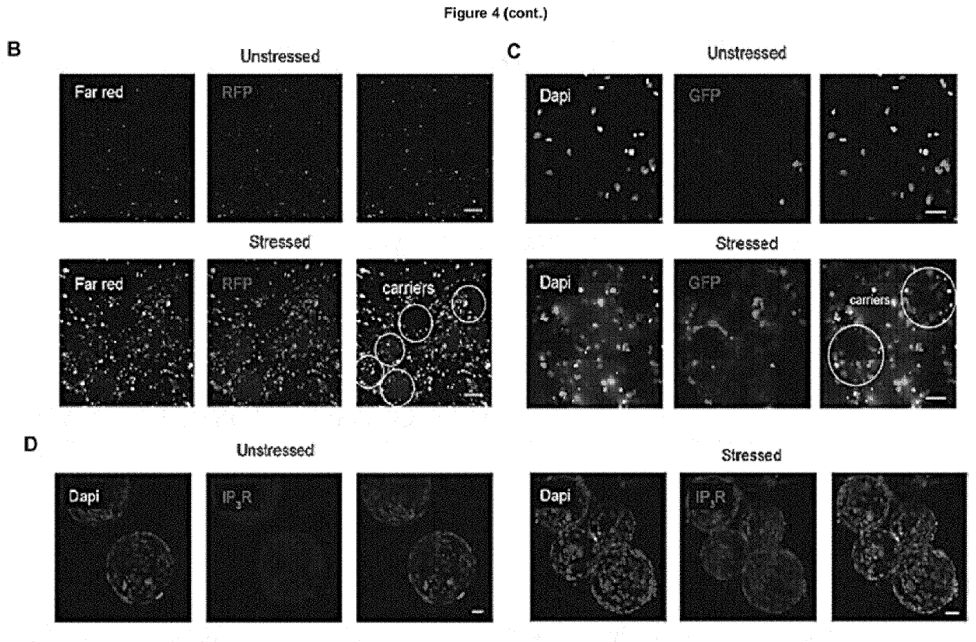

Hereto, the present inventors found that calcium signaling underlies heliocyte formation and the formation of membrane protrusions as defined elsewhere herein. In one embodiment, an increase of calcium release may be demonstrated in activated mesothelial cells (heliocytes), preferably determined by a specific calcium indicator or reporter. In another embodiment, said activated mesothelial cells may upregulate a calcium channel receptor, preferably the calcium channel receptor IP₃R, which releases calcium from the endoplasmatic reticulum (FIGS. 4B, C and D).

Additionally, the compound being used in the present disclosure may be Bepridil, Verapamil, Diltiazem, Nifedipine, Rhosin, CK-666, Golgicide A, KNK437, Quercetin, and Heat shock protein inhibitor 1 (HSPI1). The compounds Bepridil, Verapamil, Diltiazem, Quercetin, Nifedipine are calcium channel blockers, while Rhosin is a Rho GTPase Inhibitor, CK-666 is a small molecule which inhibits actin polymerization, Golgicide A is a potent, highly specific, reversible inhibitor of the cis-Golgi ArfGEF GBF1 protein. KNK437, HSPI1 and Quercetin are Inhibitors of the heat shock factor (HSF)-dependent signaling pathway. All of these cited compounds have a significant effect in reducing heliocytes and/or adhesion formation (see FIGS. 7, 9, 15 and 16).

In a preferred embodiment, the compound being used in the present invention may be selected from the group consisting of Bepridil, Verapamil, Diltiazem, Nifedipine, Rhosin, CK-666, Golgicide A, KNK437, and Quercetin.

It is also envisaged, that the reduction of the formation of heliocyte is also achieved by any combination of the abovementioned compounds. The compound thus may further be combined with a heat shock protein signaling blocker, preferably Quercetin. It is envisage by the present invention that Bepridil may be combined with heat shock protein signaling blocker such as Quercetin or KNK437. Such a combination of compounds may be Bepridil with Quercetin. It is also encompassed that Bepridil may be combined with KNK437.

It is envisage by the present invention that Verapamil may be combined with a heat shock protein signaling blocker such as Quercetin or KNK437. Further the calcium channel blocker Verapamil and Quercetin may be combined in the present invention. A combination of Verapamil and KNK437 may also be envisaged herein.

It is envisage by the present invention that Diltiazem may be combined with a heat shock protein signaling blocker such as Quercetin or KNK437. Likewise Diltiazem and Quercetin may be combined. It is further enclosed herein that Diltiazem may be combined with KN K437.

It is envisage by the present invention that Nifedipine may be combined with a heat shock protein signaling blocker such as Quercetin or KNK437. Another combination of calcium channel blocker and heat shock protein signaling blocker may be the combination of Nifedipine and Quercetin. Also Nifedipine and KNK437 are compounds which may be used together.

It is envisage by the present invention that Rhosin may be combined with a heat shock protein signaling blocker such as Quercetin or KNK437. Thus, a combination of Rhosin with Quercetin may be disclosed herein. It also means a combination of Rhosin and KNK437 may be disclosed.

It is envisage by the present invention that CK-666 may be combined with a heat shock protein signaling blocker such as Quercetin or KNK437. The small molecule CK-666 hence may be combined with Quercetin. Further CK-666 may be combined with KNK437.

It is envisage by the present invention that Golgicide A may be combined with a heat shock protein signaling blocker such as Quercetin or KNK437. Also Golgicide A and Quercetin are encompassed to may be combined. Furthermore, Golgicide A and KNK437 may be a combination of compounds envisaged by the present invention.

It is further envisage by the present invention that KNK437 may be combined with Quercetin.

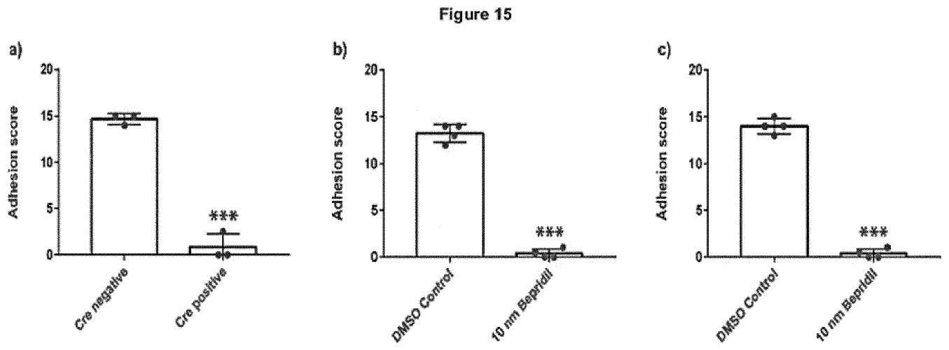

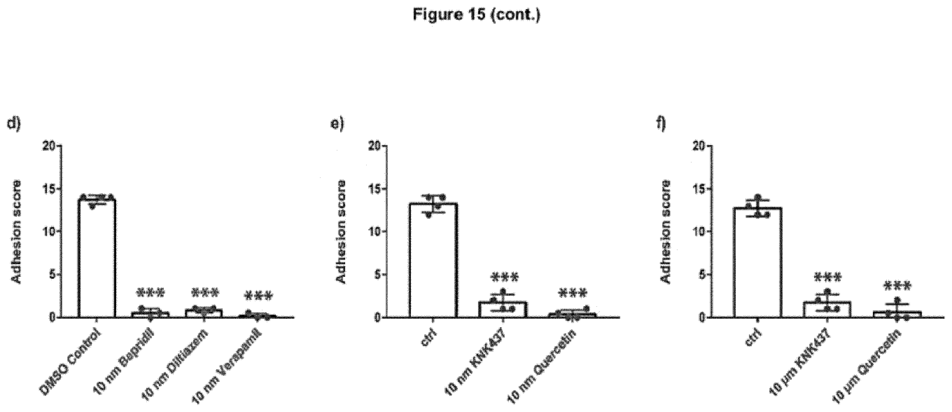

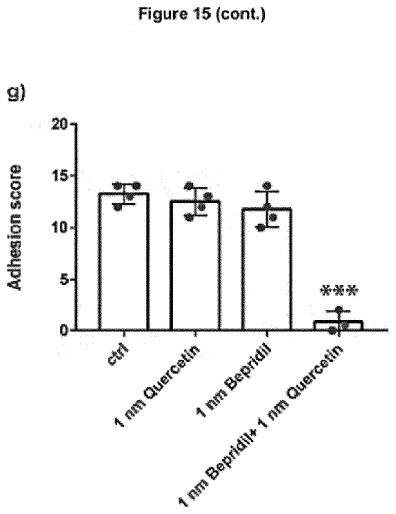

A preferred compound combination encompassed by the present invention is the combination of at least one of the calcium channel blocker Bepridil, Verapamil, Diltiazem, or Nifedipine with Quercetin, which has an additive effect on the treatment of heliocytes as described in Example 8 and shown in FIG. 15. In an even more preferred embodiment Bepridil is combined with Quercetin.

The group of compounds of the present invention consisting of Bepridil, Verapamil, Diltiazem, Nifedipine, Rhosin, CK-666, Golgicide A, KNK437, and Quercetin may be further classified. In one embodiment, the compound may be a calcium channel blocker. A preferred class of compound may be a calcium channel blocker combined with a heat shock protein signaling blocker. The calcium channel blocker is preferably selected from the groups consisting of Bepridil, Verapamil, Diltiazem, or Nifedipine. In an even more preferred embodiment, the calcium channel blocker is Bepridil. It is apparent that a calcium channel blocker is functional as blocking calcium signaling.

It is encompassed by another aspect of the invention that a calcium channel blocker is used in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain, and wherein the calcium channel blocker is preferably Bepridil. In another aspect the present invention relates to a calcium channel blocker used in a method of reducing adhesions formed by heliocytes, which comprises

a) administering to a subject an effective amount of calcium channel blocker to prevent the formation of adhesion by heliocytes after surgery or injury;
b) determining the formation of adhesion by heliocytes after the treatment with said calcium channel blocker; c) continuing the treatment if the heliocytes and/or adhesion formation by heliocytes decreased as compared to the pre-treatment.

In another aspect of the present invention a calcium channel blocker is used in a method of selecting a subject for calcium channel blocker treatment, which comprises

a) determining the heliocyte formation in a sample, wherein the sample has been obtained from a subject prior to calcium channel blocker treatment;
b) determining the heliocyte formation in a sample, wherein the sample has been obtained from the subject after treatment with the calcium channel blocker;
c) selecting the subject for continuing the calcium channel blocker treatment if the heliocyte formation is decreased in step c) as compared to step a).

In another embodiment the compound may be a heat shock protein signaling blocker, preferably selected from the group consisting of KNK437, and Quercetin. In another aspect a heat shock protein signaling blocker is used in the present invention in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain, and wherein the heat shock protein signaling blocker is preferably KNK437. In another aspect the present invention relates to a heat shock protein signaling blocker is used in a method of reducing adhesions formed by heliocytes, which comprises

a) administering to a subject an effective amount of heat shock protein signaling blocker to prevent the formation of adhesion by heliocytes after surgery or injury;
b) determining the formation of adhesion by heliocytes after the treatment with said heat shock protein signaling blocker;
c) continuing the treatment if the heliocytes and/or adhesion formation by heliocytes decreased as compared to the pre-treatment.

A heat shock protein signaling blocker can be used in another aspect of the present invention in a method of selecting a subject for heat shock protein signaling blocker treatment, which comprises

a) determining the heliocyte formation in a sample, wherein the sample has been obtained from a subject prior to heat shock protein signaling blocker treatment;
b) determining the heliocyte formation in a sample, wherein the sample has been obtained from the subject after treatment with the heat shock protein signaling blocker;
c) selecting the subject for continuing the heat shock protein signaling blocker treatment if the heliocyte formation is decreased in step c) as compared to step a).

The compound may also be a cytoskeletal remodeling blocker, preferably selected from the group consisting of Rhosin, and CK-666. It is envisaged in another aspect of the present invention that a cytoskeletal remodeling blocker is used in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain, and wherein the cytoskeletal remodeling blocker is preferably Rhosin.

Further, a cytoskeletal remodeling blocker is used in another aspect of the present invention in a method of reducing adhesions formed by heliocytes, which comprises

a) administering to a subject an effective amount of cytoskeletal remodeling blocker to prevent the formation of adhesion by heliocytes after surgery or injury;
b) determining the formation of adhesion by heliocytes after the treatment with said cytoskeletal remodeling blocker;
c) continuing the treatment if the heliocytes and/or adhesion formation by heliocytes decreased as compared to the pre-treatment.

Another aspect of the present invention relates to a cytoskeletal remodeling blocker used in a method of selecting a subject for cytoskeletal remodeling blocker treatment, which comprises

a) determining the heliocyte formation in a sample, wherein the sample has been obtained from a subject prior to cytoskeletal remodeling blocker treatment;
b) determining the heliocyte formation in a sample, wherein the sample has been obtained from the subject after treatment with the cytoskeletal remodeling blocker;
c) selecting the subject for continuing the cytoskeletal remodeling blocker treatment if the heliocyte formation is decreased in step c) as compared to step a).

Also comprised by the present invention are a pharmaceutically acceptable salt, prodrug, enantiomer, diastereomer, racemic mixture, crystalline form, amorphous, unsolved form or solvate of the compound of the present invention. Thus, the present invention may also comprise the compound of the present invention, wherein it is selected from the group consisting of Bepridil, Verapamil, Diltiazem, Nifedipine, Rhosin, CK-666, Golgicide A, KNK437, and Quercetin, or a pharmaceutically acceptable salt, prodrug, enantiomer, diastereomer, racemic mixture, crystalline form, amorphous, unsolved form or solvate thereof.

In some embodiments the compound of the present invention may be used in a method of reducing the formation of heliocytes in a subject. Even more preferably, the compound of the present invention may be used in a method of reducing the formation of heliocytes in a subject, who suffers from the early onset or the initiation phase of adhesiogenesis, the subject not yet showing pronounced/distinct adhesiogenesis when the compound of the present invention is applied to said subject.

The terms “subject,” “individual,” and “patient” are used interchangeably herein and refer to a mammal. In an embodiment, the mammal is a human. The terms “subject,” “individual,” and “patient” encompass, without limitation, individuals already exhibiting/having an existing population of heliocytes, or a risk of developing heliocytes. Subjects may be human, but also include other mammals, particularly those mammals useful as laboratory models for human disease, e.g. mouse, rat, etc.

### Pharmaceutical Compositions

In a further aspect, the present invention provides a pharmaceutical composition for use in a method of reducing the formation of heliocytes comprising at least one compound(s) as described herein and one or more pharmaceutical acceptable excipients. A pharmaceutical composition for use in a method of reducing the formation of heliocytes may comprise the compound of the present invention and/or a pharmaceutical composition per se comprising the compound of the present invention. Further, said pharmaceutical composition may comprise the compound being defined in the present invention and one or more pharmaceutically acceptable excipients. All of the abovementioned concerning the compound of the present invention may be applicable to the aspect of the pharmaceutical composition and vice versa. Further, said pharmaceutical composition may comprise one or more of the compound(s) being defined in the present invention (any two of the compounds defined, any three of the compounds defined, any four of the compounds defined, or even all five of the compounds defined) and preferably one or more pharmaceutically acceptable excipients.

In one embodiment, the pharmaceutical composition of the present invention may comprise a compound being capable of inhibiting the formation of membrane protrusions of the akropodia-type, membrane protrusions of the filopodia-type and/or membrane protrusions of the nanotube-type, a compound being capable of preventing the transmission of a mesothelial cell to an activated mesothelial cell e.g. the pathogenic phenotype of a heliocyte and/or back to a mesothelial cell and preferably one or more pharmaceutically acceptable excipients. In another embodiment the pharmaceutical composition may comprise a compound being capable of inhibiting the formation of membrane protrusions of the akropodia-type, membrane protrusions of the filopodia-type and/or membrane protrusions of the nanotube-type, a compound being capable of inducing apoptosis in a heliocyte, but not in a mesothelial cell and preferably one or more pharmaceutically acceptable excipients. In yet another embodiment, the pharmaceutical composition may comprise a compound being capable of preventing the transmission of a mesothelial cell to an activated mesothelial cell e.g. the pathogenic phenotype of a heliocyte and/or back to a mesothelial cell, a compound being capable of inducing apoptosis in a heliocyte, but not in a mesothelial cell and preferably one or more pharmaceutically acceptable excipients. In yet another embodiment, the present invention may comprise a pharmaceutical composition comprising a compound being capable of inhibiting the formation of membrane protrusions of the akropodia-type, membrane protrusions of the filopodia-type and/or membrane protrusions of the nanotube-type, a compound being capable of preventing the transmission of a mesothelial cell to an activated mesothelial cell e.g. the pathogenic phenotype of a heliocyte and/or back to a mesothelial cell, a compound being capable of inducing apoptosis in a heliocyte, but not in a mesothelial cell and preferably one or more pharmaceutically acceptable excipients.

In another embodiment, the pharmaceutical composition of the present invention may comprise a compound being capable of blocking cytoskeletal remodeling, a compound being capable of blocking protein trafficking and preferably one or more pharmaceutically acceptable excipients. Additionally, the pharmaceutical composition of the present invention may comprise a compound being capable of blocking cytoskeletal remodeling, a compound being capable of blocking calcium signaling and preferably one or more pharmaceutically acceptable excipients. Additionally, the pharmaceutical composition of the present invention may comprise a compound being capable of blocking protein trafficking, a compound being capable of blocking calcium signaling and preferably one or more pharmaceutically acceptable excipients. Additionally, the pharmaceutical composition of the present invention may comprise a compound being capable of blocking cytoskeletal remodeling, a compound being capable of blocking protein trafficking, a compound being capable of blocking calcium signaling and preferably one or more pharmaceutically acceptable excipients.

In another embodiment, the pharmaceutical composition of the present invention may comprise a compound selected from the group consisting of Bepridil, Verapamil, Diltiazem, Nifedipine, Rhosin, CK-666, Golgicide A, KNK437, and Quercetin or a pharmaceutically acceptable salt, prodrug, enantiomer, diastereomer, racemic mixture, crystalline form, amorphous, unsolved form or solvate thereof, in combination with any one of the members of the group or a pharmaceutically acceptable salt, prodrug, enantiomer, diastereomer, racemic mixture, crystalline form, amorphous, unsolved form or solvate thereof and preferably one or more pharmaceutically acceptable excipients. The pharmaceutical composition of the present invention may also comprise Bepridil, Verapamil, Diltiazem, Nifedipine, Rhosin, CK-666, Golgicide A, KNK437, and Quercetin or a pharmaceutically acceptable salt, prodrug, enantiomer, diastereomer, racemic mixture, crystalline form, amorphous, unsolved form or solvate thereof and preferably one or more pharmaceutically acceptable excipients. It may also comprise Bepridil, Verapamil, Diltiazem, Nifedipine, Rhosin, CK-666, Golgicide A, KNK437, and Quercetin or a pharmaceutically acceptable salt, prodrug, enantiomer, diastereomer, racemic mixture, crystalline form, amorphous, unsolved form or solvate thereof and preferably one or more pharmaceutically acceptable excipients.

The compound described in the present invention (in particular the compound of the present invention or a pharmaceutically acceptable salt, prodrug, enantiomer, diastereomer, racemic mixture, crystalline form, amorphous, unsolved form or solvate of the compound of the present invention) are preferably administered to a patient in need thereof via a pharmaceutical composition. In one embodiment, the pharmaceutical composition comprises a compound as described above (e.g. the compound of the present invention or a pharmaceutically acceptable salt, prodrug, enantiomer, diastereomer, racemic mixture, crystalline form, amorphous, unsolved form or solvate of the compound of the present invention) and one or more pharmaceutically acceptable excipients. In particular, any administration route, any dosage form, any dosage regimen defined for the pharmaceutical composition of the present invention may also be applied to the compound of the present invention.

The pharmaceutical composition may be administered to an individual by any route, such as enterally, parenterally or by inhalation.

The expressions “enteral administration” and “administered enterally” as used herein mean that the drug administered is taken up by the stomach and/or the intestine. Examples of enteral administration include oral and rectal administration. The expressions “parenteral administration” and “administered parenterally” as used herein mean modes of administration other than enteral administration, usually by injection or topical application, and include, without limitation, intravenous, intramuscular, intra-arterial, intrathecal, intracapsular, intraosseous, intraorbital, intracardiac, intradermal, intraperitoneal, transtracheal, subcutaneous, subcuticular, intraarticular, subcapsular, intracerebral, intracerebroventricular, subarachnoid, intraspinal, epidural and intrasternal administration (such as by injection and/or infusion) as well as topical administration (e.g., epicutaneous, or through mucous membranes (such as buccal, sublingual or vaginal)). However, the pharmaceutical composition may be administered parenterally. Even more preferably the pharmaceutical composition may be administered by injection or topically, most preferably the pharmaceutical composition may be administered topically. It is also envisaged that when a topical administration is used, the surgeon preferably applies a topical version of the compound directly on the worked-on organ surfaces (as a preventative measure) before closing up the patient. This may radically decrease the adhesion incidence.

The term “excipient” when used herein is intended to indicate all substances in a pharmaceutical composition which are not active ingredients (e.g., which are therapeutically inactive ingredients that do not exhibit any therapeutic effect in the amount/concentration used), such as, e.g., carriers, binders, lubricants, thickeners, surface active agents, preservatives, emulsifiers, buffers, flavoring agents, colorants, or antioxidants.

The pharmaceutical compositions comprising the compound of the present invention or one or more of the compound(s) being defined in the present invention may also comprise a pharmaceutically acceptable carrier. As used herein, “pharmaceutically acceptable carrier” includes any and all solvents, dispersion media, coatings, isotonic and absorption delaying agents, and the like that are physiologically compatible. The “pharmaceutically acceptable carrier” may be in the form of a solid, semisolid, liquid, or combinations thereof. If the pharmaceutical composition may be applied by injection, preferably by intraperitoneal injection, the “pharmaceutically acceptable carrier” preferably is PBS. If the pharmaceutical composition may be applied topically, the “pharmaceutically acceptable carrier” preferably is cellulose, more preferably 2% cellulose.

The pharmaceutical composition may also comprise adjuvants such as preservatives, wetting agents, emulsifying agents, pH buffering agents, and dispersing agents.

Regardless of the route of administration selected, the pharmaceutical composition used according to the present invention, may be formulated into pharmaceutically acceptable dosage forms by conventional methods known to those of skill in the art (cf., e.g., Remington, “The Science and Practice of Pharmacy” edited by Allen, Loyd V., Jr., 22^{nd} edition, Pharmaceutical Sciences, September 2012; Ansel et al., “Pharmaceutical Dosage Forms and Drug Delivery Systems”, 7^{th} edition, Lippincott Williams & Wilkins Publishers, 1999).

A pharmaceutical composition can be administered by a variety of methods known in the art. As will be appreciated by the skilled artisan, the route and/or mode of administration will vary depending upon the desired results. The pharmaceutical compositions containing one or more active compounds can be prepared with carriers that will protect the one or more active compounds against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such compositions are generally known to those skilled in the art. See, e.g., Sustained and Controlled Release Drug Delivery Systems, J. R. Robinson, ed., Marcel Dekker, Inc., New York, 1978.

To administer a compound used in the present invention by certain routes of administration, it may be necessary to coat the compound with, or co-administer the compound with, a material to prevent its inactivation. For example, the compound may be administered to an individual in an appropriate carrier, for example, liposomes, oil or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7: 27(1984)).

Pharmaceutical compositions typically are sterile and stable under the conditions of manufacture and storage.

Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the individuals to be treated; each unit contains a predetermined quantity of active compound calculated to produce the desired therapeutic effect in association with the required pharmaceutical carrier. When the pharmaceutical composition is administered topically, a low dose is sufficient to be used, preferably administering said low dose once. In this context, a low dose refers to dosages in the nanomolar range. When the pharmaceutical composition is administered by injection (e.g. intraperitoneal injection), higher doses and several injections per week may be used.

Actual dosage levels of the active ingredients in the pharmaceutical compositions used according to the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds used according to the present invention at levels lower than that required in order to achieve the desired therapeutic effect and gradually increase the dosage until the desired effect is achieved. In general, a suitable daily dose of a composition used according to the present invention will be that amount of the compound which is the lowest dose effective to produce a therapeutic effect. Such an effective dose will generally depend upon the factors described above. It is preferred that administration be intraperitoneal, topical intranasal, oral, intravenous, intramuscular, or subcutaneous. It is even more preferred that administration be intraperitoneal or topical, most preferably topical. If desired, the effective daily dose of the pharmaceutical composition may be administered daily, or as two, three, four, five, six or more sub-doses administered separately at appropriate intervals throughout the day, optionally, in unit dosage forms. While it is possible for a compound used according to the present invention to be administered alone, it is preferable to administer the compound as a pharmaceutical formulation/composition.

The pharmaceutical composition used according to the present invention can be formulated for parenteral administration by injection, for example, by bolus injection, or continuous infusion or topical administration.

Formulations for injection can be presented in units dosage form (e.g., in phial, in multi-dose container), and with an added preservative. The pharmaceutical composition used according to the present invention can take such forms as suspensions, solutions or emulsions in oily or aqueous vehicles, and can contain formulatory agents such as suspending, stabilizing, or dispersing agents. Alternatively, the agent can be in powder form for constitution with a suitable vehicle (e.g., sterile pyrogen-free water) before use. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachets indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Dosage forms for the topical administration of the pharmaceutical composition used according to the present invention may include a powder, spray, ointment, paste, cream, lotion, gel, solution, patch, preferably a gel. The active compound(s) of the pharmaceutical composition may be mixed under sterile conditions with a pharmaceutically acceptable carrier, and with any preservatives, buffers, or propellants which may be required.

The pharmaceutical composition used according to the invention can also, if desired, be presented in a pack, or dispenser device which can contain one or more unit dosage forms containing the active compound. The pack can for example comprise metal or plastic foil, such as blister pack. The pack or dispenser device can be accompanied with instruction for administration.

The pharmaceutical composition described herein may also be used in an in vitro method. Such an in vitro method encompassed in another aspect of the present invention is a method for detecting the presence of heliocytes forming adhesions in a subject, which comprises:

a) providing a sample obtained from a subject, said sample comprising one or more cell(s);
b) seeding a plurality of cells of a subject in the in vitro bead assay as described herein;
c) contacting said cells with
i) the compound as described herein and/or,
ii) the pharmaceutical composition as described herein; and
   c) detecting the presence of heliocytes forming adhesions in the cells seeded in said in vitro bead assay, wherein the detection of heliocytes forming adhesions is indicative of heliocytes forming adhesions in the subject.

Additionally, the present invention may include a method of reducing the formation of heliocytes in a subject comprising administering an effective amount of said compound of the present invention to a subject in need thereof.

Also comprised by the present invention is the use of the compound of the present invention for the manufacture of a medicament for reducing the formation of heliocytes.

All of the abovementioned concerning the compound of the present invention may be applicable to the aspect of the method of reducing the formation of heliocytes and the use of said compound for the manufacture of a medicament and vice versa.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. The term “at least one” refers to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the following examples, offered for illustrative purposes only. The examples are not intended to limit the scope of the present invention in any way.

The invention is further characterized by the following items:
1. A compound for use in a method of reducing the formation of heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
2. The compound for the use of item 2, wherein a mesothelial cell is activated by hypoxia, ischemia, inflammation, infection, a chemical stimulus, desiccation, a mechanical trauma, cold shock, heat shock, osmotic shock, or a foreign body to become an activated mesothelial cell.
3. The compound for the use of any one of the preceding items, wherein a heliocyte is characterized by membrane protrusions of the akropodia-type, and/or membrane protrusions of the filopodia-type.
4. The compound for the use of any one of the preceding items, wherein a heliocyte is characterized by vesicle and/or exosome secretion.
5. The compound for the use of any one of the preceding items, wherein heliocytes develop adhesions.
6. The compound for the use of any one of the preceding items, wherein the development of adhesions by heliocytes results in adhesiogenesis.
7. The compound for the use of item 6, wherein adhesiogenesis is inter- or intra-organ adhesiogenesis.
8. The compound for the use of any one of the item 5 to 7, wherein inter- or intra-organ adhesiogenesis occurs postoperative.
9. The compound for the use of any one of the preceding items, wherein the compound is capable of preventing the transmission of a mesothelial cell to the pathogenic phenotype of a heliocyte and/or capable of inducing apoptosis in a heliocyte, but not in a mesothelial cell.
10. The compound for the use of any one of the preceding items, wherein the compound blocks cytoskeletal remodeling, blocks protein trafficking, blocks calcium signaling, or blocks heat shock protein signaling.
11. The compound for the use of any one of the preceding items, wherein the compound is selected from the group consisting of Bepridil, Verapamil, Diltiazem, Nifedipine, Rhosin, CK-666, Golgicide A, KNK437, and Quercetin.
12. The compound for the use of any one of the preceding items, wherein the compound is selected from the group consisting of Bepridil, Verapamil, Diltiazem, Nifedipine, Rhosin, CK-666, Golgicide A, KNK437, Quercetin and HSPI1.
13. The compound for the use of item 10 to 12, wherein the compound is further combined with a heat shock protein signaling blocker, preferably Quercetin.
14. The compound for the use of item 11, wherein the compound is a calcium channel blocker selected from the group consisting of Diltiazem, Verapamil, Nifedipine and Bepridil.
15. The compound for the use of item 12, wherein the compound is a heat shock protein signaling blocker selected from the group consisting of KNK437, HSPI1 and Quercetin.
16. The compound for the use of item 12, wherein the compound is a heat shock protein signaling blocker selected from the group consisting of HSPI1 and Quercetin.
17. The compound for the use of item 11, wherein the compound is a cytoskeletal remodeling blocker selected from the group consisting of Rhosin, and CK-666.
18. The compound for the use of any one of the preceding items, wherein the use comprises administering the compound of any one of the items 10 to 17 after surgery or injury, determining the adhesion formation by heliocytes and continuing the compound treatment if the adhesion formation by heliocytes decreased as compared to the pre-treatment.
19. An in vitro bead assay for analyzing heliocytes and/or the formation of adhesions, comprising the steps of
   a) seeding mesothelial cells onto a coated dish and letting said cells grow to a monolayer;
   b) coating carrier beads with mesothelial cells;
   c) activating said cells coated on said carrier beads of step b) with a stimulus,
   d) seeding said activated cells coated on said carrier beads of step b) and c) onto the monolayer of step a);
   and analyzing the activated mesothelial cells on said carrier beads, or analyzing the activated mesothelial cells eluted from said carrier beads.
20. The in vitro bead assay of item 19, wherein the mesothelial cells are preferably Met-5A positive, before seeded in step a) and/or coated in step b).
21. The in vitro bead assay of item 19, wherein the activated cells on said carrier beads of step c) and/or step d) are capable of fusing the cell-coated beads together and can optionally be selected by size.
22. The in vitro bead assay of item 19, wherein the stimulus of step c) is selected from the group consisting of hypoxia, ischemia, inflammation, infection, a chemical stimulus, desiccation, a mechanical trauma, cold shock, heat shock, osmotic shock, or a foreign body.
23. The in vitro bead assay of item 19 to 22, further comprises
   i) contacting said activated cells coated on said carrier beads after step c) with a compound; and
   ii) determining the effect of the compound on the activated mesothelial cells and/or formation of adhesions after step d).
24. The compound for the use of item 9, wherein said capability of said compound is determined by the in vitro bead assay of item 19 to 23.
25. The in vitro bead assay of item 19 to 23 in use for determining the capability of a compound to
   a) prevent the transmission of a mesothelial cell to the pathogenic phenotype of a heliocyte; and/or
   b) induce apoptosis in a heliocyte, but not in a mesothelial cell
   c) prevent the formation of adhesion and/or adhesiogenesis.
26. An in vitro method for determining the formation of heliocytes, wherein the method comprises obtaining a sample comprising mesothelial cells from a subject, preparing the sample according to the in vitro bead assay of item 19 to 23 and determining the formation of heliocytes in said in vitro bead assay.
27. An in vitro method for treating heliocytes and/or adhesions formed by heliocytes, wherein the method comprises obtaining a sample comprising mesothelial cells from a subject, preparing the sample according to the in vitro bead assay of item 19 to 22 and treating the heliocytes and/or adhesions formed by heliocytes by contacting said heliocytes with a compound according to item 23.
28. A calcium channel blocker for use in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain, and wherein the calcium channel blocker is preferably Bepridil.
29. The calcium channel blocker for the use of item 28, comprising
   a) administering to a subject an effective amount of calcium channel blocker to prevent the formation of adhesion by heliocytes after surgery or injury;
   b) determining the formation of adhesion by heliocytes after the treatment with said calcium channel blocker;
   c) continuing the treatment if the heliocytes and/or adhesion formation by heliocytes decreased as compared to the pre-treatment.
30. A heat shock protein signaling blocker for use in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain, and wherein the heat shock protein signaling blocker is preferably KNK437.
31. The heat shock protein signaling blocker for the use of item 30, comprising
   a) administering to a subject an effective amount of heat shock protein signaling blocker to prevent the formation of adhesion by heliocytes after surgery or injury;
   b) determining the formation of adhesion by heliocytes after the treatment with said heat shock protein signaling blocker;
   c) continuing the treatment if the heliocytes and/or adhesion formation by heliocytes decreased as compared to the pre-treatment.
32. A cytoskeletal remodeling blocker for use in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain, and wherein the cytoskeletal remodeling blocker is preferably Rhosin.
33. The cytoskeletal remodeling blocker for the use of item 32, comprising
   a) administering to a subject an effective amount of cytoskeletal remodeling blocker to prevent the formation of adhesion by heliocytes after surgery or injury;
   b) determining the formation of adhesion by heliocytes after the treatment with said cytoskeletal remodeling blocker;
   c) continuing the treatment if the heliocytes and/or adhesion formation by heliocytes decreased as compared to the pre-treatment.
34. A pharmaceutical composition for use in a method of reducing the formation of heliocytes, comprising at least one compound(s) of item 10 to 17 and one or more pharmaceutically acceptable excipients.
35. An in vitro method for detecting the presence of heliocytes forming adhesions in a subject, comprising:
   a) providing a sample obtained from a subject, said sample comprising one or more cell(s);
   b) seeding a plurality of cells of a subject in the in vitro bead assay of item 19 to 23;
   c) contacting said cells with
      i) the compound according to item 10 to 17 and/or,
      ii) the pharmaceutical composition according to item 34; and
   c) detecting the presence of heliocytes forming adhesions in the cells seeded in said in vitro bead assay, wherein the detection of heliocytes forming adhesions is indicative of heliocytes forming adhesions in the subject.
36. A method of selecting a subject for calcium channel blocker treatment, comprising
   a) determining the heliocyte formation in a sample, wherein the sample has been obtained from a subject prior to calcium channel blocker treatment;
   b) determining the heliocyte formation in a sample, wherein the sample has been obtained from the subject after treatment with the calcium channel blocker;
   c) selecting the subject for continuing the calcium channel blocker treatment if the heliocyte formation is decreased in step c) as compared to step a).
37. A method of selecting a subject for heat shock protein signaling blocker treatment, comprising
   a) determining the heliocyte formation in a sample, wherein the sample has been obtained from a subject prior to heat shock protein signaling blocker treatment;
   b) determining the heliocyte formation in a sample, wherein the sample has been obtained from the subject after treatment with the heat shock protein signaling blocker;
   c) selecting the subject for continuing the heat shock protein signaling blocker treatment if the heliocyte formation is decreased in step c) as compared to step a).
38. A method of selecting a subject for cytoskeletal remodeling blocker treatment, comprising
   a) determining the heliocyte formation in a sample, wherein the sample has been obtained from a subject prior to cytoskeletal remodeling blocker treatment;
   b) determining the heliocyte formation in a sample, wherein the sample has been obtained from the subject after treatment with the cytoskeletal remodeling blocker;
   c) selecting the subject for continuing the cytoskeletal remodeling blocker treatment if the heliocyte formation is decreased in step c) as compared to step a).
39. Bepridil for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
40. Verapamil for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
41. Diltiazem for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
42. Nifedipine for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
43. CK-666 for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
44. Golgicide A for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
45. KNK437 for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
46. Quercetin for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.
47. Rhosin for use in a method of reducing heliocytes and/or adhesions, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.

### EXAMPLES OF THE INVENTION

### Material and Methods

Cell Culture.

Met-5A cells were cultivated in 10% FBS (Sigma Aldrich, #F9665), F199 (Sigma Aldrich, #M4530), 18 ng/mL EGF (R&D systems, #236-EG), 400 nM Hydrocortisone (Sigma Aldrich, #H4001), 16 ng/mL Insulin-Transferrin-Selenium (Gibco, #41400045), 10 mM HEPES (Gibco, #15630080), 2.5 mg/L Amphotericin (Gibco, #5000980), Trace elements B (Corning, #15343641), 50 units Penstrep (Gibco, #15070063). After hypoxic shock, cells were cultured in ‘assay medium’: 2% FBS, 10 mM HEPES, Trace elements B and 50 units Penstrep. Cells were cultivated on 2% gelatin (Sigma Aldrich, #G1393) coated dishes. Cells were passaged using PBS and Trypsin EDTA (Sigma Aldrich, #T4049).

Stable Cell Lines.

Cells were transfected with corresponding PiggyBac- and Helper plasmids using Lipofectamine 2000 (Invitrogen, #11668) according to the manufacturer's instructions. After 48 h the medium was replaced with 10 ng/mL Puromycin (Tebu-Bio, #BIA-P1230) containing medium. Every other day medium was replaced, up until two weeks of selection, after which transgenes were stably expressed.

Luciferase and Nanoluciferase Measurement.

Cells were incubated with ice cold luciferase lysis buffer (25 mM Tris-HCL pH 7.8, 1% Triton X-100, 15 mM MgSO₄, 4 mM EGTA, 1 mM DTT) for 20 minutes. Both assays were performed in a 96-wells plate format. Luciferase firefly substrate was dissolved in PBS, and consisted of 20 mM Tricine, 2.67 mM MgSO₄, 0.1 mM EDTA, 33.3 mM DTT, 0.52 mM ATP, 0.27 mM Acetyl-CoA, 5 mM NaOH, 50 mM MgCO₃, and 0.47 mM D-Luciferin (Carl Roth, #CN24.3). For nanoluciferase, the substrate solution included 47.2 μM Coelenterazine (Carl Roth, #4094.4). Luminescence was detected after 5 minutes of substrate and lysate co-incubation using the TriStar² LB 942 Modular Multimode microplate reader (Berthold Technologies).

In Vitro Assays.

### High-Throughput Carrier-Carrier Adhesion Assay

Met-5A cells were seeded together with Cytodex® 3 microcarrier beads (Sigma Aldrich, #03275) in a ratio of 500:1, and allowed to adhere and grow for 5 days. Cell-covered beads were then eluted from the culture dish using a 25 mL stripette. The resulting solution was strained through a 100 μm cell strainer (Corning, #352360). Hypoxic shock was induced by placing the bead-containing cell strainer under a running cell culture flow hood for 15 minutes. Afterwards, beads were eluted with assay medium and placed in a cultivation dish coated with HEMA silicate solution (Sigma Aldrich, #P3932), to prevent cell attachment. After indicated time points, carrier complexes were collected by filtering them through a 200 μm cell strainer, which allows individual carriers to pass, while trapping carrier complexes. Adhesions were measured in a high throughput manner through the use of Met-5A cells stably expressing luciferase (AF23) or nanoluciferase (AF1) using Integra Viaflo pipettes.

### High-Throughput Carrier-Monolayer Adhesion Assay

Met-5A cells were seeded on gelatin (Sigma Aldrich, #G1393) coated dishes. Once a confluent monolayer was established, stressed cell-covered beads were seeded onto the monolayer. For the inhibitory experiments, if not indicated otherwise, monolayer cells were pretreated with the indicated compound for 30 minutes in culture medium. Afterwards the adhesion assay was performed as described above.

### Cre-Exchange Transmission Assay

Met-5A cells stably expressing Cre-recombinase (AF32) were seeded on Cytodexe 3 microcarrier beads (Sigma Aldrich, #03275) and were exposed to hypoxic shock as described, and placed on a Met-5A monolayer stably expressing the dTomato-P2A-NanoLuciferase reporter construct (AF34). Nanoluciferase luminescence was measured after 48 hours. This represents the initial stress-dependent transmission.

Wild-type Met-5A cells were then seeded on a monolayer, exposed to hypoxic shock, and placed together with unstressed cell-covered beads that stably express Cre-recombinase (AF32) for 3 hours. Carriers were then separated from the monolayer and placed on an unstressed Met-5A monolayer stably expressing the dTomato-P2A-NanoLuciferase reporter construct (AF34). Nanoluciferase luminescence was measured after 48 hours. This represents the first stress-independent transmission.

Wild-type cell-covered beads were then exposed to hypoxic shock, and seeded on an unstressed wild-type Met-5A monolayer for 3 hours. Carriers were then separated from the monolayer and placed on unstressed cell-covered beads stably expressing Cre-recombinase (AF32) for 3 hours. Carriers were then isolated once more and placed on an unstressed Met-5A monolayer stably expressing the dTomato-P2A-NanoLuciferase reporter construct (AF34). Nanoluciferase luminescence was measured after 48 hours. This represents the second stress-independent transmission.

This procedure was continued similarly for the consecutive third and fourth transmissions.

### Adhesion Propagation Assay

Met-5A cells stably expressing nanoluciferase (AF1) were seeded on Cytodexe 3 microcarrier beads (Sigma Aldrich, #C3275) and were exposed to hypoxic shock as described, and placed on a wild-type Met-5A monolayer. After 24 hours, unbound carriers were washed away and nanoluciferase luminescence was measured. This represents the initial stress-dependent transmitted adhesion.

Wild-type Met-5A cells were then seeded on a monolayer, exposed to hypoxic shock, and placed together with unstressed cell-covered beads that stably express nanoluciferase (AF1) for 3 hours. Carriers were then separated from the monolayer and placed on an unstressed Met-5A wild-type monolayer. After 24 hours, unbound beads were washed away and nanoluciferase luminescence was measured. This represents the first stress-independent transmitted adhesion.

Wild-type cell-covered beads were then exposed to hypoxic shock, and seeded on an unstressed wild-type Met-5A monolayer for 3 hours. Carriers were then separated from the monolayer and placed on unstressed cell-covered beads stably expressing nanoluciferase (AF1) for 3 hours. Carriers were then isolated once more and placed on an unstressed Met-5A monolayer stably expressing the dTomato-P2A-NanoLuciferase reporter construct (AF34). Unbound beads were then washed away and nanoluciferase luminescence was measured after 24 hours. This represents the second stress-independent transmitted adhesion.

This procedure was continued similarly for the consecutive third and fourth transmissions.

### Spinning Disc Cell Detachment Force Assay

The protocol used to measure cell detachment forces was adapted from¹³. In brief, Met-5A cells stably expressing nanoluciferase were seeded on gelatin (Sigma Aldrich, #G1393) coated glass slides. After two days, cells were stressed as described above and further grown for another 24 hours. Slides were then rotated for 5 min at 4000 rpm, lysed, and nanoluciferase activity was measured. For baseline values, cell detachment forces were expressed relative to nanoluciferase values derived from a lysed non-rotated glass slide on which cells were grown to confluence. For carrier to monolayer detachment, the same protocol was performed with a Met-5A cell monolayer seeded on gelatin coated glass slides on which Met-5A coated Cytodex® 3 microcarrier beads (Sigma Aldrich, #03275) stably expressing nanoluciferase were added. To derive meaningful values in terms of generated forces, values were used as described in table 1.1¹³, which describes the relationship between the spinning speed (rpm), and the wall shear stress for a range of radial positions across the spinning disc (from the axis of rotation).

### Apoptosis Assay

Met-5A cells were seeded on gelatin (Sigma Aldrich, #G1393) coated dishes. Once a confluent monolayer was established, stressed cell-covered beads were seeded onto the monolayer. For the inhibitory experiments, if not indicated otherwise, monolayer cells were pretreated with 10 μM of indicated compounds for 30 minutes in culture medium. After 24 h fragmented DNA was determined via Cell Death Detection ELISAPLUS (Sigma Aldrich, #11774425001).

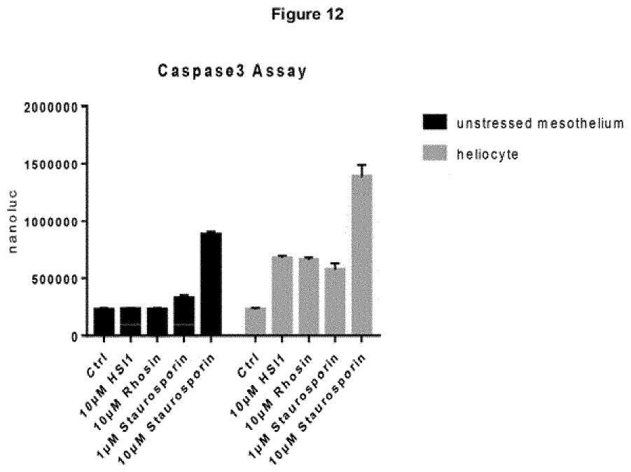

Then, determination of caspase3 activity in heliocytes was performed. Cells stably expressing a nanoluciferase-caspase3-cleaving-site-degredation-peptide (FIG. 12) were grown on beads seeded onto a Met-5A monolayer. After 24 h nanoluciferase activity was determined. The degradation peptide leads to constitutive degradation of nanoluciferase, active caspase3 cleaves the protein linker, thus removing the degradation signal, leading to increased nanoluciferase amount.

Scanning Electron Microscopy.

Met-5A cells were seeded on Cytodex® 3 microcarrier beads (Sigma Aldrich, #C3275) and exposed to hypoxic shock as described. Cell-covered beads were then added to Met-5A cells seeded on gelatin (Sigma Aldrich, #G1393) coated glass slides, and after the indicated time points glass slides were fixed 0/N at 4° C. using 3% glutaraldehyde and 0.1% sodium cacodylate buffer, pH 7.4 (Electron Microscopy Sciences, #16538). For animal tissues, adhesions were induced in mice as described, and sacrificed 16 hours later. The peritoneum was then fixed in the same manner as the glass slide samples. Samples were dehydrated in a serial dilution of ethanol and dried by the critical-point method, using CO₂ as the transitional fluid (Polaron Critical Point Dryer CPC E3000; Quorum Technologies, Ringmer, UK). Samples were sputter coated with a 7 nm layer of platinum by a sputtering device (Emitech K575; Quorum Technologies) and observed by scanning electron microscopy (JSM 6300F; JEOL, Eching, Germany).

Imaging.

### Murine Tissue Preparation for Imaging Purposes

Upon organ excision, organs were fixed overnight at 4° C. in 2% formaldehyde. The next day, fixed tissues were washed three times in Dulbecco's phosphate buffered saline (DPBS, GIBCO, #14190-094), and depending on the purpose, either embedded, frozen in optimal cutting temperature compound (Sakura, #4583) and stored at −20° C., or stored at 4° C. in PBS containing 0.2% gelatin (Sigma Aldrich, #G1393), 0.5% Triton X-100 (Sigma Aldrich, #X100) and 0.01% Thimerosal (Sigma Aldrich, #T8784) (PBS-GT).

### 3D Imaging of Whole Mount Tissue Samples

Whole-mount samples were stained and cleared with a modified 3DISCO protocol³⁶. In short, samples stored in PBS-GT were incubated with primary antibodies in PBS-GT with shaking, for 36 hours at RT. Excessive antibody was removed by thorough washing in PBS-GT for 6-12 hours and refreshing the solution every 1-2 hours. Incubation with fluorophore-coupled secondary antibodies (Molecular Probes) in PBS-GT for 36 hours was followed by thorough washing in PBS-GT as described above. When necessary, samples were dehydrated in an ascending Tetrahydrofuran (Sigma, #186562) series (50%, 70%, 3×100%; 60 minutes each), and subsequently cleared in dichloromethane (Sigma, #270997) for 30 min and eventually immersed in benzyl ether (Sigma, #108014). Non-cleared samples were imaged in 35 mm glass-bottom dishes (Ibidi, #81218) using a laser scanning confocal microscope (Zeiss LSM710) or SP8 Multi-photon microscope (Leica). Cleared samples were imaged whilst submerged in benzyl-ether with a light-sheet fluorescence microscope (LaVision BioTec).

### 3D Multiphoton Imaging

For multi-photon imaging, samples were embedded in a 4% NuSieve GTG agarose solution (Lonza, #50080). Imaging was performed using a 25× water-dipping objective (HC IRAPO L 25x/1.00 W) coupled to a tunable pulsed laser (Spectra Physics, Insight DS+). Multi-photon excited images were recorded with external, non-descanned hybrid photo detectors (HyDs). Following band pass (BP) filters were used for detection: HC 405/150 BP for DAPI/Hoechst and Second Harmonic Generation (SHG), a ET 525/50 BP for green channel, 585/40 BP for red channel and a 650/50 BP (magenta) for far red. Tiles were merged using Leica Application suite X (v3.3.0, Leica) with smooth overlap blending and data were visualized with Imaris software (v9.1, Bitplane).

### 3D Lightsheet Imaging

Whilst submerged in benzyl-ether, specimens were illuminated on two sides by a planar light-sheet using a white-light laser (SuperK Extreme EXW-9; NKT Photonics). EdU and PDPN were excited at 640/30 nm and 576/23 nm respectively and the emitted light was detected using 690/50 nm and 620/31 nm filters. Optical sections were recorded by moving the specimen chamber vertically at 5-mm steps through the laser light-sheet. Three-dimensional reconstructions were obtained using Imaris imaging software (v9.1, Bitplane).

### 2D Imaging of Murine and Human Tissue Sections

Fixed tissues were embedded in optimal cutting temperature (OCT) and cut with a Microm HM 525 (Thermo Scientific). Sagittal cross-sections of 7 μm were used for analyses. In short, sections were fixed in ice-cold acetone for 5 min at −20° C., and then washed with PBS. Sections were then blocked for non-specific binding with 1% BSA and 5% goat serum in PBS for 60 minutes at room temperature, and then incubated with primary antibody in 1% BSA and 5% goat serum in PBS, 0/N at 4° C. The next day, following washing, sections were incubated in PBS with fluorescent secondary antibody, for 120 min at RT. Finally, sections were washed and incubated with Hoechst 33342 nucleic acid stain (Invitrogen, #H1399), washed in ddH₂O, mounted with Fluoromount-G® (Southern Biotech, #0100-01), and stored at 4° C. in the dark.

### Masson's Trichrome Staining

To visualize deposited matrix, Masson's trichrome staining was performed (Sigma Aldrich, #HT15). In brief, samples were fixed for 10 min in ice cold acetone at −20° C., and subsequently washed in dH₂O for 5 min. Then, samples were incubated overnight in Bouin's solution (Sigma Aldrich, #HT10132) at room temperature, and washed the next day under running tap water for 5 min. Samples were then immersed in Weigert's iron hematoxylin solution (Sigma Aldrich, #HT1079) for 3 min, and again washed under running tap water for 5 min. Samples were incubated with Briebrich Scarlet-Acid Fuchsin solution for 5 min, rinsed in dH₂O, and incubated with Phosphotungstic/Phosphomolybdic acid solution for 5 min. Finally, samples were immersed in Aniline Blue solution for 10 min, washed in 1% acetic for 2 min, and further washed with dH₂O, and then dehydrated through an ethanol gradient. Samples were then dipped 8-10 times and cleared in Roti®-Histol (Carl Roth, #6640) and mounted with Roti®-Histokitt (Carl Roth, #6638).

### Imaging of Met-5A Covered Carrier-Carrier Complexes

Carrier-carrier complexes where fixed in 4% PFA in PBS for 20 minutes at RT. Afterwards, complexes were washed two times with PBS. Cells were permeabilized for 10 minutes in 0.1% Triton X-100 (Sigma Aldrich, #X100) in PBS at 4° C., after which they were washed two times with 0.02% Tween-20 (Sigma Aldrich, #9416) in PBS. Carriers were then blocked for non-specific binding with 5% BSA and 0.02% Tween-20 in PBS for 60 minutes at 4° C. on a rocking platform, and then incubated with primary antibody in 0.02% Tween-20 in PBS 0/N at 4° C. The next day, following washing, carriers were incubated in PBS with fluorescent secondary antibody for 120 min at 4° C. on a rocking platform. Finally, carriers were washed and incubated with Hoechst 33342 nucleic acid stain (Invitrogen, #H1399).

### Imaging of Met-5A Carrier-to-Monolayer Samples

For carrier-to-monolayer samples Met-5A cells where seeded on gelatin (Sigma Aldrich, #G1393) coated glass slides. Samples were fixed with 4% PFA in PBS for 20 minutes at RT. Cells were permeabilized for 10 minutes in 0.1% Triton X-100 (Sigma Aldrich, #X100) in PBS at 4° C., after which they were washed two times with 0.02% Tween-20 (Sigma Aldrich, #9416) in PBS. Slides were then blocked for non-specific binding with 5% BSA and 0.02% Tween-20 in PBS for 60 minutes at 4° C., and then incubated with primary antibody in 0.02% Tween-20 in PBS 0/N at 4° C. The next day, following washing, carriers were incubated in PBS with fluorescent secondary antibody for 120 min at 4° C. Finally, slides were washed and incubated with Hoechst 33342 nucleic acid stain (Invitrogen, #H1399).

### Membrane Dye Labeling of Microcarrier and Monolayer Cultures

Met-5A cells were labeled with DiO staining solution (Invitrogen, #V22886) according to the manufacturer's instructions, and seeded as a monolayer on gelatin (Sigma Aldrich, #G1393) coated glass chambers (Ibidi, #80287). After 3 days of cultivation, a separate population of cells were seeded together with Cytodex® 3 microcarrier beads (Sigma Aldrich, #C3275), and labeled using the PKH26 Red Fluorescent Cell Linker kit (Sigma Aldrich, #MINI26-1KT). Cell-covered beads were then exposed to hypoxic shock as described, and were added to the monolayer culture.

Image Analyses.

### Pre-Processing

All image processing and analyses were performed with exported Tif images using Fiji (ImageJ 2.0.0/1.52c, USA). Fluorescent channels were split and the brightness and contrast were adjusted to reduce background in order to prevent misinterpretation of background as cellular structures in the segmentation step.

### Pre-Processing: Segmentation

Mesothelial protrusion analysis was performed using the Advanced weka segmentation Fiji plugin³⁷. It utilizes a collection of machine learning algorithms for segmentation. Specifically, pixel-based segmentation is based on image features annotated to different classes. Pixel samples were free drawn and assigned to respective classes, e.g. ‘filopodia’, ‘cell body’, or ‘background’. Subsequent rounds of training were performed to allot respective pixels and structures to its corresponding classes for improved segmentation. Training features, such as ‘Gaussian blur’, ‘Sobel filter’, ‘Hessian’, ‘Difference of Gaussians’ and ‘Membrane projections’ were applied along with default classifier ‘Fast Random Forest’. Other settings were kept as default (membrane thickness 1, membrane patch size 19, minimum sigma 1.0, and maximum sigma 16.0). The trained classifier and data were saved for analysis of other ‘stressed’ and ‘unstressed’ mesothelial datasets. A macro was written to automate the above steps for other datasets with pause for 5 seconds (wait(5000) function) after each step for smooth processing. Images from the segmented classes were extracted and subjected to post processing.

### Post Processing: Total Filopodial Surface Area

To quantify the total filopodial surface area, filopodial segments were obtained as described in ‘Segmentation’. Brightness and contrast was adjusted from these images, and were converted into binary images. Mean fluorescent intensity was then computed.

### Post Processing: Length and Width of Filopodia

Length and width of filopodial protrusions were calculated using the ImageJ plugin ‘Ridge detection’³⁸. In short, filopodial segments were obtained as described in ‘Segmentation’. ‘Correct position’, ‘estimate width’, ‘extend line’, ‘display results’, and ‘add to manager’ settings were selected. Parameters used included optional parameters (line width: 10, high contrast: 230, low contrast: 87) and mandatory parameters (Sigma: 3.39, lower threshold: 0.51, upper threshold: 1.19, minimum line length: 15.00). Parameters were optimized using preview function for a single dataset and similar values and settings were applied for other datasets. Values of length and width were extracted from the summary tab and exported as an Excel file.

### Skeletal Analysis

Skeletal analysis was performed using the Fiji plugin ‘Skeletonize’. It utilizes iterative erosion of structures until only the skeleton remains. In brief, the ‘cell body’ and ‘filopodia’ segments described in ‘Segmentation’ were merged and flattened. These images were converted into binary images and skeletonize function was applied.

### Directionality

Directionality of the protrusions (geographical map) was performed using the Fiji plugin ‘Directionality’. It computes the angle of the structures to infer the preferred orientation in the input image. It produces a histogram indicating the amount of structures in a given direction. Isotropic images are expected to give a flat histogram whereas, anisotropic images, produces peaks at respective orientation. Directionality plugin produces histograms from 0° to 180°. In order to obtain values in 360°, the used image was fragmented into 6 (2×3) smaller pictures to obtain 180° in clockwise and anticlockwise directions. Directionality function was applied to each of these images and values of degrees and amounts were exported as an excel-file. The histogram was converted into a polar coordinate plot using “ggplot2” package in R. Individual plots were overlaid in Adobe Photoshop CC 2015 to obtain a full geographical map.

In vivo EdU labeling.

Animals were injected intraperitoneally with 1 μg of EdU (Invitrogen, #A10044) dissolved in 100 μL PBS on the day of surgery, and sacrificed on day 5. Following organ excision and fixation overnight in 2% formaldehyde, EdU was visualized using the Click-iT™ EdU Alexa Fluor™ 647 imaging kit (Invitrogen, #C10340), according to the manufacturer's instructions. The Click-iT® reaction cocktail was incubated with the samples for 36 hours at room temperature to allow sufficient penetration into the tissue. Tissues were then further processed according to the whole-mount imaging protocol (see ‘3D imaging of whole mount tissue samples’).

Localized Treatment with Topical Cellulose.

Small molecule inhibitors were solubilized in sterile 2% hydroxyethyl-cellulose (Sigma Aldrich, #09368). Compounds were being added immediately prior to surgery, and were derived from a 100-150 mM stock solution to minimize the final DMSO content. The final solution (200 μL per 30 g body weight) was sandwiched between the visceral and parietal layer of the injured cecum and peritoneum respectively.

Animals.

All animal experiments were conducted under strict governmental and European guidelines and were approved by the local government for the administrative region of Upper Bavaria, under license number 55.2-1-54-2532-150-2015. Pathogen-free male and female C57BL/6 mice (6-10 week old) were obtained from Charles River and group-housed in climate-controlled quarters with a 12 h/12 h light/dark cycle. Animals were allowed food and water ad libitum.

Murine Adhesion Model.

Mice were anesthetized by an intraperitoneal injection of a Medetomidin (500 μg/kg), Midazolam (5 mg/kg) and Fentanyl (50 μg/kg) cocktail, hereafter referred to as MMF. Monitoring anesthetic depth was assessed by toe reflex. Eyes were covered with Bepanthen to avoid dehydration, and the abdomen was shaved and disinfected with betadine and sterile phosphate buffered saline (PBS). Animals were kept on their backs on a heating plate at 37° C. A midline laparotomy (1-1.5 cm) was performed through the skin and peritoneum. Four hooks, positioned around the incision and fixed to a retractor and magnetic base plate, allowed for clear access to the abdominal cavity. A small surgical brush was used to gently abrade the peritoneal surface and apposing cecal surface. Two surgical knots using 4-0 silk sutures (Ethicon) were then placed through the serosal surface of the peritoneum. A cotton swab was used to gently apply a dab of talc powder (Sigma Aldrich, #243604) onto the injured surfaces. Before closure of the incision, buprenorphine (0.1 mg/kg) was pipetted in the abdomen to allow for initial post-surgical analgesia. For long-term analgesia, metamizol (Novalgin, 200 mg/kg) was provided through daily injection. The peritoneum and skin were closed with two separate 4-0 silk sutures (Ethicon). Upon closure of the incision, mice were woken up by antagonizing the MMF solution through a subcutaneous cocktail injection of Atipamezol (1 mg/kg) and Flumazenil (0.25 mg/kg). Mice were allowed to recover on a heating pad, after which they were housed together (females) or individually (males), and followed for 1-5 days. Adhesions were scored using gross morphological features that indicated adhesion development. Five individual adhesion features were scored (see table below) that together provided a cumulative value that determined the total adhesion score. With this system, complete absence of adhesions were scored as 0, whereas the maximum adhesion score was 15.

Scoring Table 1 (cumulative value determines total adhesion score).

| | | | | | | | | | Adhesion | |
|---|---|---|---|---|---|---|---|---|---|---|
| | | | | | | | | | supported by | |
| NO. of attached | | NO. of attached | | Total adhesive | | | | | gravity or mild | |
| structures | | secondary organs | | coverage | | | Attached colon? | | pulling strength? | |
| Outcome | Score | Outcome | Score | Outcome | | Score | Outcome | Score | Outcome | Score |
| 0 | 0 | 0 | 0 | 0 | cm² | 0 | No | 0 | No | 0 |
| 1 | 1.5 | 1 | 1 | 0-0.5 | cm² | 1 | Yes | 3 | Yes | 3 |
| 2 | 3 | 2 | 2 | 0.5-1.0 | cm² | 2 | | | | |
| | | 3 | 3 | >1.0 | cm² | 3 | | | | |

Human Tissue.

All human samples have been obtained during surgery at the Department of Surgery, Klinikum rechts der Isar, Technical University of Munich, following approval of the local ethics committee of the Technical University of Munich, Germany (NO. 173/18 S). Adhesions were intraoperatively diagnosed and dissected from the respective organs and prepared for further analysis.

Single Cell RNA Sequencing (Drop-Seq).

Samples were incubated for 5 minutes in Trypsin-EDTA solution at 37° C. Trypsin was inactivated with ice cold assay medium and cells were washed twice with ice cold PBS. Drop-seq experiments were performed as described previously^{18,19}, with few adaptations during the single cell library preparation. Briefly, single cells were diluted in PBS, supplemented with 0.04% bovine serum albumin up to a final concentration of 100 cells/μL. Using a microfluidic PDMS device (Nanoshift), single cells were co-encapsulated in droplets with barcoded beads (Chemgenes Corporation, Wilmington, Mass.) at a final concentration of 120 beads/μL. Droplets were collected for 15 min/sample. After droplet breakage, beads were harvested, washed, and prepared for on-bead mRNA reverse transcription (Maxima RT, Thermo Fisher). Following an exonuclease I (New England Biolabs) treatment for the removal of unused primers, beads were counted, aliquoted (2000 beads/reaction, equals ˜100 cells/reaction), and pre-amplified by 13 PCR cycles (primers, chemistry, and cycle conditions identical to those previously described in Macosko et al., 2015). PCR products were pooled and purified twice using 0.6× clean-up beads (CleanNA). Prior to tagmentation, cDNA samples were loaded on a DNA High Sensitivity Chip on the 2100 Bioanalyzer (Agilent) to ensure transcript integrity, purity, and amount. For each sample, 1 ng of pre-amplified cDNA from an estimated 1000 cells was tagmented by Nextera XT (Illumina) with a custom P5 primer (Integrated DNA Technologies). Single cell libraries were sequenced in a 100 bp paired-end run on the Illumina HiSeq4000 using 0.2 nM denatured sample and 5% PhiX spike-in. For priming of read 1, 0.5 μM Read1CustSeqB was used (primer sequence; SEQ ID NO. 1: GCCTGTCCGCGGAAGCAGTGGTATCAACGCAGAGTAC).

### Bioinformatic Processing of Single Cell RNA Sequencing Data

The Drop-seq core computational pipeline was used for processing next generation sequencing reads of the scRNA-seq data, as previously described¹⁸. Briefly, STAR (version 2.5.2a) was used for mapping³⁹. Reads were aligned to the hg19 genome reference (provided by Drop-seq group, GSE63269). For barcode filtering, barcodes were excluded with less than 200 genes detected. A high proportion (>10%) of transcript counts derived from mitochondria-encoded genes may indicate low cell quality, and unqualified cells were removed from the downstream analysis. After obtaining the digital gene expression (DGE) data matrix, Seurat for dimension reduction, clustering and differential gene expression analysis was used¹⁸.

### Principal Component Analysis

Using only variable genes, a principal component analysis (PCA) was performed. The top 15 principal components were used as input for the Seurat FindClusters function at a resolution of 0.5. This method accomplishes a clustering of the cells by embedding them in a graph like structure. A k-nearest neighbor graph is used, in which any two cells (represented as nodes) that are connected by an edge have an edge weight that is among the k smallest distances from the first node to any other. Thus, edges are drawn between cells with similar gene expression patterns. Modularity optimization methods such as the Louvain Algorithm try to reveal parts of the graph with different connectivity and therefore divide the graph into separate interconnected modules.

### Partition Based Graph Abstraction Method

To visualize the clustering result of the high dimensional single-cell data, the Fruchterman-Reingold algorithm from the Python toolkit Scanpy was employed²⁰. Additionally, to display the connectivity between the cell groups the partition based graph abstraction (PAGA) method was used²⁰. The cells were grouped according to the time point of extraction. In the graph, those groups are represented as nodes and edges between the nodes show the connectivity or relatedness of these groups, therefore quantifying their similarity with respect to gene expression differences.

### Time Resolved Pathway Analysis

To predict the activity of pathways and cellular functions based on the observed gene expression changes, the Ingenuity® Pathway Analysis platform was used (IPA®, QIAGEN Redwood City, www.qiagen.com/ingenuity) as previously described⁴⁰. The analysis uses a suite of algorithms and tools embedded in IPA for inferring and scoring regulator networks upstream of gene-expression data based on a large-scale causal network derived from the Ingenuity Knowledge Base. Using the ‘Downstream Effects Analysis’⁴¹ embedded in IPA aimed at identifying those biological processes and functions that are likely to be causally affected by up- and down-regulated genes in the single cell transcriptomics dataset. In the analysis genes with an overlap P-value of >7 (log 10) that had an activation Z-score >2 were considered as activated and those with an activation Z-score <−2 as inhibited.

Statistical Analyses.

All data represent the mean±SEM. A Shapiro-Wilk's test (p>0.05) as well as visual inspection of the respective histograms, normal Q-Q plots and box plots were used to test whether samples were normally distributed (approximately), using IBM SPSS Statistics version 23. Two group comparisons were made using an unpaired Student's t-test for normally distributed data or a Mann-Whitney U test as the non-parametric equivalent. Comparisons between three or more groups were performed using a one-way ANOVA followed by Tukey's post hoc test for normally distributed data, or with a Kruskal-Wallis H test for non-normally distributed data. A value of p<0.05 was considered statistically significant, where * is p<0.05, ** is p<0.01, and *** is p<0.001. Analyses were performed with GraphPad Prism version 6

(GraphPad Software, Inc.). Directionality/Polar-coordinates plot was performed using the “ggplot2” library #1234 in R #5678 Version 3.4.1, 2^{42,43}.

### Example 1: Adhesion Modeling In Vitro

In vitro assay to explore adhesions between adjacent organ surfaces by coating specialized commercial microcarrier beads with a monolayer of human mesothelial Met-5A cells was created (FIG. 1A). Adhesions are thought to occur during open abdominal surgery through exposure to irritants, such as tissue desiccation, foreign bodies (e.g. talcum powder), and pockets of hypoxic ischemia caused by surgical ties¹². Desiccation-induced adhesions in vitro assay was modelled by exposing coated beads to ambient air for 15 minutes. Strikingly this led the beads to cluster into large aggregates (FIG. 1C). Repeating the experiment over a monolayer of unstressed mesothelial Met-5A cells resulted in carriers adhering tightly to the monolayer within just 60 minutes. The developing carrier aggregates continuously recruited more carriers over time, until plateauing in adherence capacity at approximately 72 hours (FIG. 1D).

To assess bead fusion in a high-throughput manner, cells stably expressing nanoluciferase or luciferase were generated, that were then coated on beads and added to a wild-type monolayer prior to stress (FIG. 2A). Both desiccation and exposure to low concentrations of talcum powder dramatically increased the measured luminescence signal (FIGS. 2B and C). Stressed luciferase-expressing carriers were then mixed with unstressed nanoluciferase-expressing carriers and purified carrier-carrier complexes (FIG. 2D). Under these conditions, stressed carriers actively incorporated unstressed nanoluciferase carriers into large carrier aggregates (FIG. 2E), indicating adhesions can spread to healthy surfaces.

Scanning electron microscopy images of stressed carriers revealed that fusions between carriers developed by filopodial protrusions that bridged carrier-carrier complexes or carriers with the monolayer (FIG. 1E and FIG. 2F). The morphologies of the carriers themselves (which are normally round) flattened or buckled, likely due to the strong adhesive and tensile forces that were exerted by the activated mesothelium across the carrier surfaces. To further probe these intercellular protrusions, unstressed and stressed cells were cultured on a more physiological Matrigel matrix bedding. Under these conditions, stressed cells developed many long protrusions that bridged nearby colonies, effectively forming a vast network of intercellular connections (FIG. 2G). Contrary, unstressed cells remained as separate colonies. To measure the tensile forces generated by these membrane bridges, cell detachment forces were measured through hydrodynamic shear using a spinning disc device¹³. Detachment force to separate bound carriers was already measurable 8 hours after desiccation stress, and saturated around 24 hours (FIG. 2H). Unstressed cells did not cluster and were thus not measurable. To assess the effect of membrane protrusions on the binding capabilities of mesothelium, the adherence of cells cultured as a monolayer was measured. Upon stress, the binding force increased more than 3-fold compared to unstressed cells (FIG. 2I). For comparison, this observed increase in binding force is greater than that of myofibroblastic transformation in highly contractile human hypertrophic scars¹⁴. Interestingly, stress-induced aggregation was entirely attributable to membrane protrusions, and did not require any matrix deposition.

### Example 2: Transformed Mesothelial Cells Transmit Adhesion Pathology Through Cytoskeletal Protrusions

To study the dynamics of mesothelium's membrane protrusions in higher detail, two stable cell lines from Met-5A mesothelial cells were generated, expressing either the membrane-bound fluorescent marker dTomato or GFP, and coated beads and monolayers with either one of them. After administering a 15-minute desiccation shock, the ensuing fluorescence patterns were followed by live confocal imaging over 48 hours (data not shown). Stressed mesothelial cells rapidly broke free from existing junctional networks and acquired a highly dynamic cytoskeleton with various forms of membrane protrusions that continuously probed their surroundings (FIG. 3A). Numbers and subtypes of membrane projections on each cell was in constant flux, from thin fine spicules to large amoeboid fragments, which occasionally shed from stressed cells (data not shown). When coated on beads, protrusions were observed interacting with opposing beads to establish a tether, pulling beads together. Conversely, unstressed cultures maintained a typical mesothelial cobblestone appearance and lacked any membrane extensions (FIG. 3A). Due to their dramatic shift in morphology and close resemblance to microbial heliozoa, these stressed cells were called ‘heliocytes’¹⁵. A machine learning algorithm was developed to characterize the heliocyte cell state based on the extent of membrane protrusions across entire cell surfaces (FIG. 4A). The total filopodial area was more than 6-fold higher in heliocytes compared to unstressed mesothelium (FIG. 3B). Each heliocyte developed several main filopodial branches from which many secondary protrusions branched out (FIG. 3C). Multiple known forms of protrusions, including (branched) filopodia, and nanotubes co-developed within individual cells (FIG. 3D). An additional unreported protrusion type was commonly expressed in heliocytes, where a single large protrusion formed a cell body that yielded several smaller extensions (FIG. 3D). Due to its hand-like morphology, these were named ‘akropodia’, from the Greek word rakros' (extremity, e.g. hands). The typical surface area of akropodia averaged 20-25% of its main cell body (FIG. 3E).

Strikingly, in the live confocal video (data not shown), unstressed GFP+ cells were frequently observed to express dTomato upon membrane contact with dTomato+ stressed cells, suggesting exchange of cellular contents occurs during these early stages. To irrefutably prove transmissibility of adhesions and cytosolic exchange between mesothelial cells, two series of experiments were designed to address these questions individually. First, repeated consecutive mixing experiments were performed with nanoluciferase-expressing Met-5A cells to study the extent of adhesion propagation. When unstressed cells were exposed to heliocytes, and then separated, they were fully able to generate adhesions even though they had not themselves experienced stress (FIG. 3F). Pharmacological inhibition of HIF-1α signaling has been one of the few proposed intervention routes to reduce adhesion burden¹⁶, thus it was tested to see whether the HIF-1α inhibitor echinomycin could inhibit transmissibility. Interestingly, whereas the initial desiccation-induced transmission could be prevented by HIF-1α inhibition, all subsequent transmissions could not, demonstrating that the initial shock sets an irreversible signaling pathway into motion (FIG. 3G). Transmission occurred rapidly, as unstressed cells only had to be exposed to heliocytes for a total of 90 min for successful transmission to occur and allow them to form adhesions themselves (FIG. 3H). Second, two additional cell lines were generated, one expressing Cre recombinase and one expressing a stop codon flanked by two LoxP sites followed by a dTomato-P2A-Nanoluciferase. In this transmission assay, Cre recombinase only drives dTomato-P2A-Nanoluc expression if extensive cytoplasmic mixing occurs that allows Cre recombinase protein to be transferred and shuttled into the nucleus of the dTomato-P2A-Nanoluc cells. Indeed, extensive dTomato fluorescence was observed 3 hours after unstressed carriers were seeded onto a stressed monolayer (FIG. 3I). Under consecutive mixing conditions, like the propagation set-up, naive cells receiving cellular contents from heliocytes could in turn transmit to other cells independently from HIF-1α signaling, for a total of three rounds after the original stress stimulus (FIGS. 3J and K). Because cytoskeletal networks and membrane shuffling are extremely sensitive to shifts in available calcium¹⁷, it was asked whether calcium signaling underlies heliocyte formation and filipodial extensions, using two independent approaches.

In the first approach, a human mesothelial cell line was generated that permanently expresses the green fluorescent calcium indicator GCaMP6s. Then beads with these cells were coated and stressed them with a short stress stimulus.

In a second approach, Met-5A cells with the permeable calcium reporter X-Rhod-1 were labelled and preceded as before. Both experiments revealed calcium release at fusion sites (FIGS. 4B and C). Immuno-labelling confirmed that stressed carrier aggregates up-regulated the calcium channel receptor IP₃R (FIG. 4D). The above experiments demonstrate that adhesions can spread across organ surfaces and between organs through the active transmission of heliocyte characteristics, which likely occurs through active membrane fusion and calcium-dependent signal transduction. Furthermore, the data show that this key patho-cellular event precedes and occurs independent of matrix deposition and inflammation, and as a direct response to stress.

### Example 3: Single-Cell Transcriptomic Analysis of Heliocyte Formation

To determine the transcriptional program that transforms healthy mesothelial cells into heliocytes, subtle gene expression changes were analyzed by performing highly parallel genome-wide expression profiling of individual mesothelial cells (single-cell RNA-seq) using the Dropseq workflow¹⁸ (FIG. 6A). >16,000 cells were sequenced from Met-5A mesothelial cells at sequential time points after a desiccation shock, as well as under control unstressed conditions. Using unique molecular identifier barcode counting¹⁹, 20,027 genes were quantified and principal component analysis was performed with the count levels of 7942 genes with the biggest difference between the groups. Subtle transcriptional changes were analyzed within the cell population by constructing k-nearest neighbor graphs using the Fruchterman-Reingold algorithm²⁰. Interestingly, stressed cells clustered separately from unstressed cells after 8 hours and onwards. Most cells from later time points clustered together, suggesting that major transcriptional changes occurred during the first 8 hours post induction.

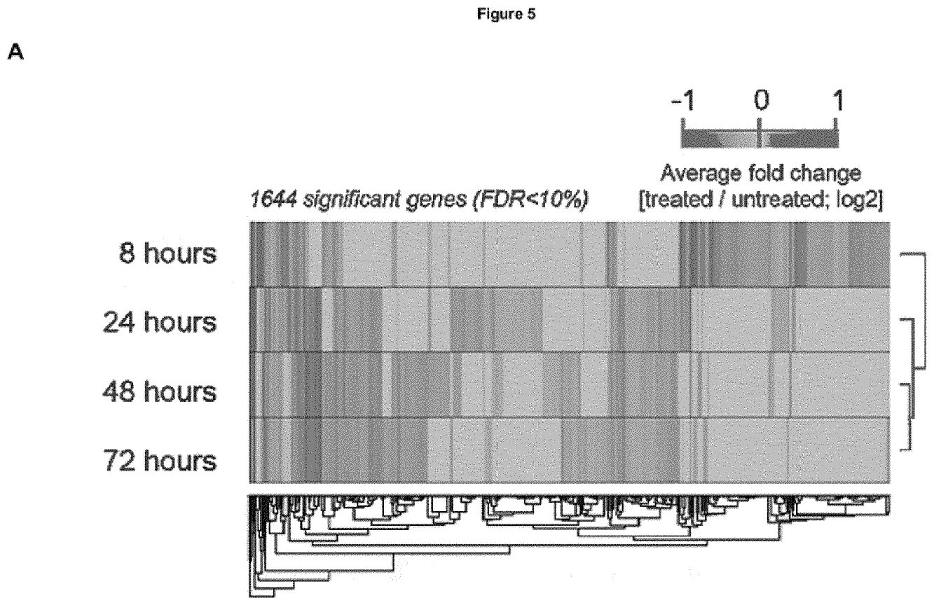

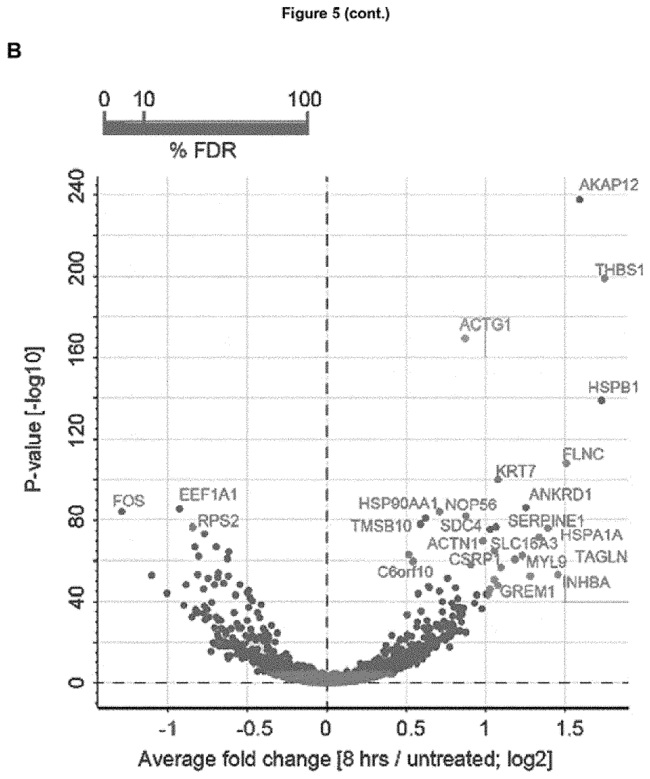

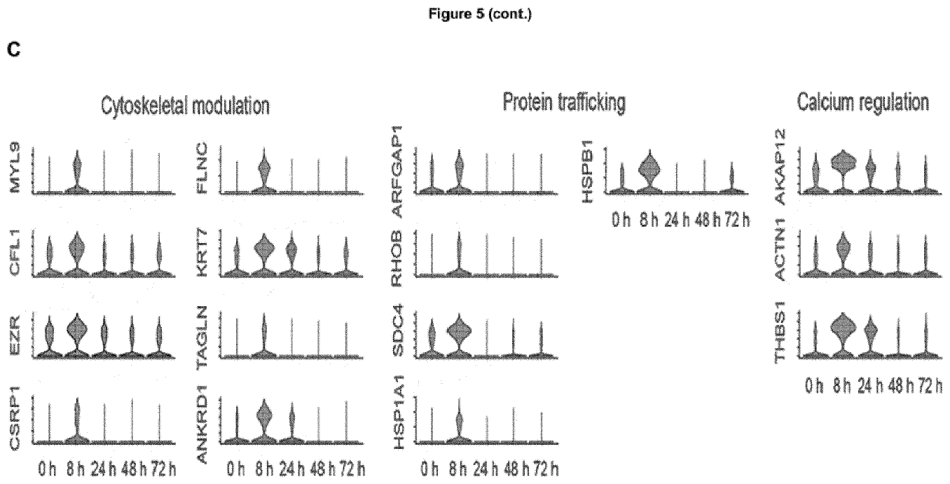

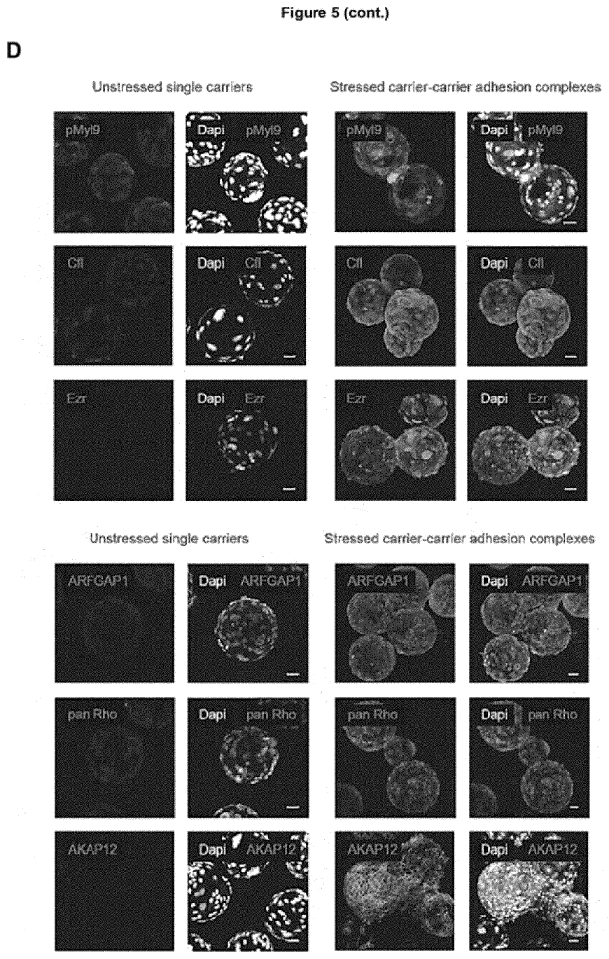

In order to identify underlying mechanisms of heliocyte transformation, differential gene expression analysis was performed using a likelihood-ratio test for single cell gene expression data²¹. This identified 1644 genes that were regulated in stressed cells at one or more time points (FDR<10%). In accordance with previous results, differentially expressed genes of the 8 hour cluster adopted a unique gene signature compared to later time points (FIG. 5A). Therefore all subsequent efforts were focused on this time window. Several of the most differentially expressed genes included the A-kinase anchor protein 12 (AKAP12), Heat shock protein beta-1 (HSPB1), Actin Gamma 1 (ACTG1), and Thrombospondin-1 (THBS1), which are known to interact and modulate the actin cytoskeletal network or calcium homeostasis (FIG. 5B). Gene pathway analyses and downstream effects analysis (IPA) of the 8 hour cluster highlighted cell invasion programs mediated by Rho-family GTPases, actin cross-linkers, and modification of adherens junction proteins as characteristics of the early heliocyte program. To further elaborate on this program, genes that peaked at 8 hours after stress were assayed, and dipped at subsequent time points (FIG. 5C). Close inspection on individual gene functions allowed us to partition these genes into three distinct hierarchies: 1) actin cross-linkers and cytoskeletal modulators, 2) protein traffickers, and 3) calcium regulators. Importantly, whole bead 3D immuno-labelling confirmed all of the tested gene products were highly expressed in heliocyte-covering beads, and virtually absent in unstressed cells (FIG. 5D). To analyze the functional involvement of these genes in heliocyte transformation and adhesion formation, selective small-molecule inhibitors were added to media containing stressed beads and assessed adhesion formation and transmissibility using the nanoluciferase assays as described above. As expected, treatment with Bepridil (calcium channel blocker), Rhosin (inhibits the Rho-GEF binding domain), CK-666 (inhibitor of the Arp2/3 complex and actin assembly), and Golgicide A (inhibits Arf1 and protein trafficking) completely blocked protrusion development in heliocytes (FIG. 6B), and as a consequence adhesion formation and transmissibility (FIGS. 6C, D and E).

### Example 4: Blocking Cytoskeletal Protrusions Inhibits Adhesion Formation

Scanning electron microscopy images of peritoneal adhesion tissue revealed in vivo that mesothelial cells transformed at sites of injury to heliocytes as early as 16 hours after injury. Heliocytes were functionally identifiable based on the following markers: ADP-ribosylation factor GTPase-activating protein 1 (ARF-GAP1, a Golgi-associated enzyme that regulates protein trafficking²³), pan Rho GTPase (a family of well-known G proteins that control intracellular actin dynamics and cytoskeletal programming²⁴), MYL9 (a calcium-sensitive regulatory protein that is necessary for cytoskeletal dynamics²⁵), and AKAP12 (a compartmentalizing protein that localizes at the membrane and is regulated by intracellular calcium²⁶). These markers were uniquely expressed on heliocytes, and absent on naive mesothelium (FIG. 7A, FIG. 8A-C).

Next, the fate of injured mesothelium was traced to irrefutably prove that heliocytes originate directly from mesothelial cells in vivo. The previously generated RNA-seq dataset derived from murine peritoneal mesothelium was used¹¹, and protein C receptor (PROCR) was found to be highly expressed. As PROCR expression is additionally elevated in several mesothelial cancers^{27,28}, Procr^{CreERT2-IRES-tdTomato} knock-in mice were used, in which a CreERT2-IRES-tdTomato cassette is inserted after the first ATG codon of Procr²⁹. These mice were then crossed with a reporter Rosa26^{mTmG} line to mark all Procr descendants as GFP positive. Consequently, 5 days after adhesion induction, heliocytes were seen between the fused parietal and visceral layers, thus proving mesothelial descendance of heliocytes in vivo.

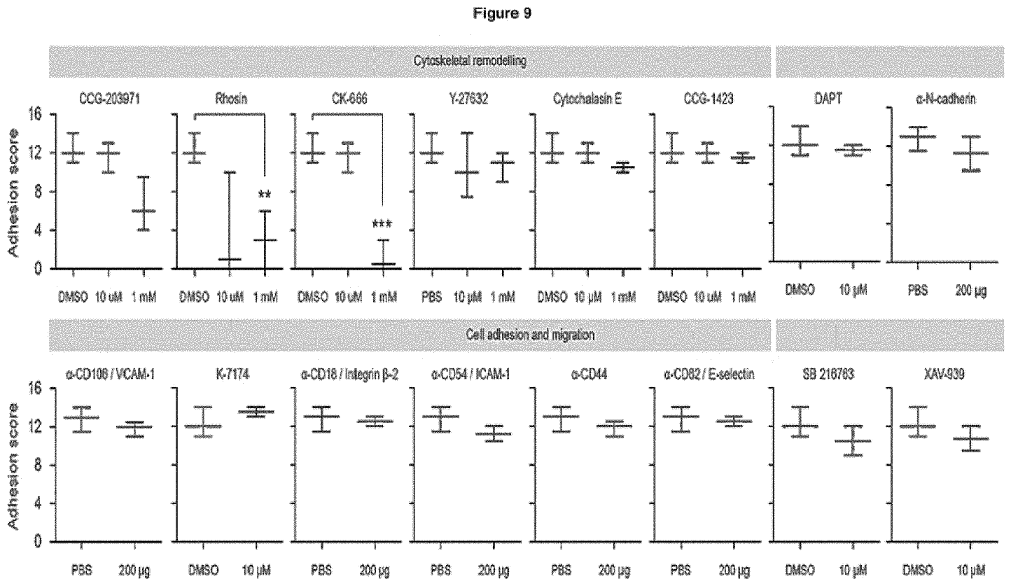

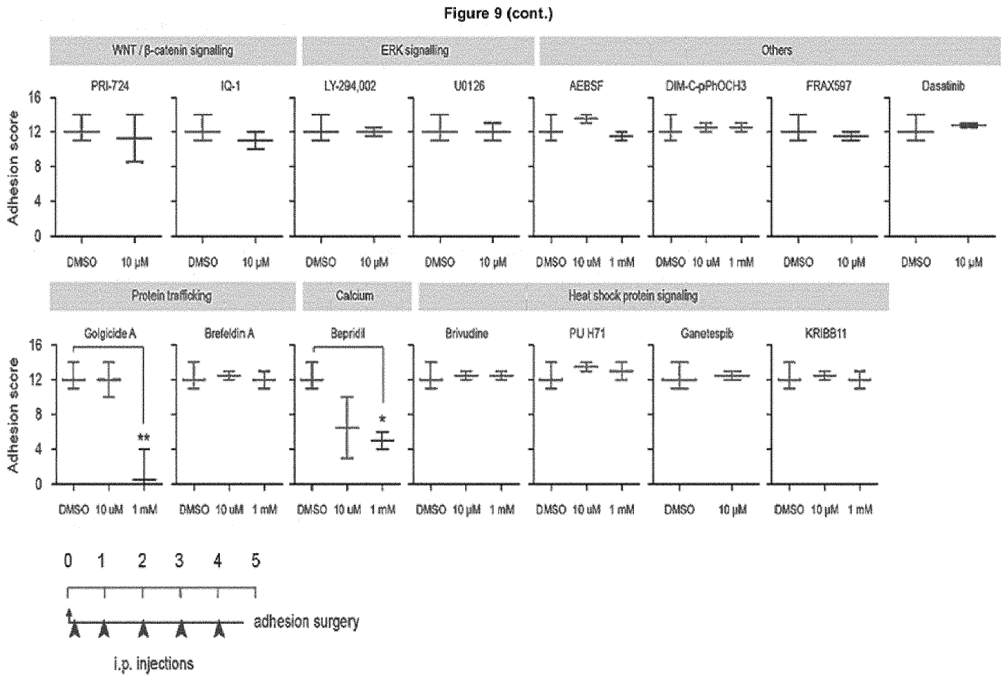

Next, the role of heliocyte formation was functionally determined in adhesion pathogenesis in vivo. To determine the specificity of the findings, a panel of 9 small-molecule inhibitors was employed that selectively target cytoskeletal effectors, protein traffickers, and calcium regulators, and additionally screened 22 other individual transcriptional and signaling pathway targets, including WNT, Notch, and ERK signaling. Following adhesion induction, mice received compounds through daily intraperitoneal injection, and were sacrificed at day 5 for analysis. The cytoskeletal modulators CK-666 and Rhosin, the protein trafficking blocking compound Golgicide A, and the calcium channel antagonist Bepridil all robustly inhibited adhesion formation, whereas inhibition of a broad range of signaling targets through the other 22 compounds did not. These results confirm the in vitro findings that cellular protrusions drive adhesion pathogenesis (FIG. 9). The findings that heliocytes transmit cell transformation to expand the adhesion foci led to reason that early and localised administration of the target molecules should be sufficient to prevent the early onset of adhesions. Thus each of the above four compounds were topically applied through a viscous solution of 2% cellulose that was placed immediately after injury onto the injured area alone. Indeed, a single early and localized administration effectively and completely inhibited adhesion formation in animals, indicating that heliocytes are the active agents of pathologic adhesion transmission (FIG. 7B).

Based on the above, a comprehensively revised model of adhesion formation was propose (FIG. 10), wherein: (i) injury induces the transformation of mesothelium into heliocytes, (ii) filopodial bridges from heliocytes tether organs, and (iii) transduce similar cell transformations to nearby cells thereby propagating the adhesion foci across apposing surfaces. (iv) Organ fusions are independently followed by extensive matrix deposition to form a mature scar across both organ surfaces.

### Example 5: Apoptosis Assay

In order to test listed inhibitor treatments for potential unwanted side effects, in vitro as well as in vivo apoptosis readouts were performed. Tissues of treated mice showed enhanced amounts of fragmented DNA at the prevented adhesion sites compared to more remote regions. Next, fragmented DNA amounts in the in vitro adhesions were determined, staurosporine representing as the positive control. Stressed mesothelial cells treated with 10 μM of Rhosin and HSI1 showed significantly more fragmented DNA (FIG. 11). Apoptosis signaling cascades dependent on caspase3 protease activity. To screen caspase3 activity in a high throughput manner, a new functional screening assay was developed. The assay is based on a nanoluciferase fused to a degradation signaling peptide interlinked with caspase3 cleavage sites. Once activated, caspase3 cleaves protein linker, removes the degradation signal and prevents proteasomal digestion of the nanoluciferase. Stressed mesothelial cells stably expressing the reporter construct show enhanced nanoluciferase activity upon treatment with Staurosporine (positive control). Rhosin and heat shock protein inhibitor 1, demonstrating higher caspase3 activity in treated heliocytes (FIG. 12).

### Example 6: Characterization of Heliocyte Derived Exosomes Via Mass Spectrometry

In vitro bead assay was performed as described above, culture medium was collected from stressed cells at 24 h post adhesion formation and subjected to two consecutive centrifugations to remove residual cells and cellular debris: 800 g for 10 min and 12,000 g for 30 min at 4° C. The pellet was discarded, and the supernatant was passed through a 0.22-μM pore diameter filter (Millipore), followed by ultracentrifugation at 110,000 g for 3 h at 4° C. using TFT80.2 Ti rotor (Wx Ultra 90, Thermo Scientific). The ultracentrifuged pellet was collected from the bottom of the tube, resuspended in sterile filtered PBS, and subsequently ultracentrifuged at 110,000 g for 3 h again. Finally, the exosomal pellet was resuspended proteolytic digested and analyzed on a Q-Exactive mass spectrometer.

### Example 7: Heliocyte Derived Exosomes Transmit Adhesion Formation In Vitro

In vitro bead assay was performed as described above, instead of stressing mesothelial cells under the hood, mesothelial cells were treated with exosomes derived from unstressed or stressed mesothelial cells. Exosomal pellet was generated as described above (FIG. 14).

### Example 8: Mesothelia Cells are Precursors of Adhesion and Different Compounds Prevent Adhesion Formation

To demonstrate the role of mesothelial cells in adhesion formation, the inventors crossed PROCR mice with Rosa26tm1(DTA) mice to selectively ablate PROCR+ mesothelial cells upon tamoxifen administration.

Adhesions completely failed to develop in mice treated with a single dose of tamoxifen immediately after surgery, whereas genotype negative animals generated full blown adhesions (FIG. 15A). These results prove that mesothelial cells are precursors of protruding cells and of adhesions in vivo.

To test whether the chosen one-shot treatment regime blocks adhesions in the long term, the inventors studied mice 2 months after surgery.

In fact, no adhesions were detectable even 2 months post-surgery after calcium channel inhibition (FIG. 15B). Next, the inventors showed that intra peritoneal injection (after wound closure) of calcium channel inhibitors also prevented adhesion formation (FIG. 15C). In addition, they could demonstrate the robustness of the inventor's therapeutic route by applying other calcium channel inhibitors (FIG. 15D).

Next, the inventors aimed at another branch of the mesothelial adhesion program they identified. By inhibiting the heat shock factor (HSF)-dependent signaling pathway, the inventors successfully inhibited adhesion formation via cellulose-based and one-time oral application (FIGS. 15E and F).

Finally, the inventors showed that combined inhibition of calcium channels and HSF additively affects adhesion inhibition (FIG. 15G).

### Example 9: A Single Application of 10 nM Nifedipine Prevents Adhesion

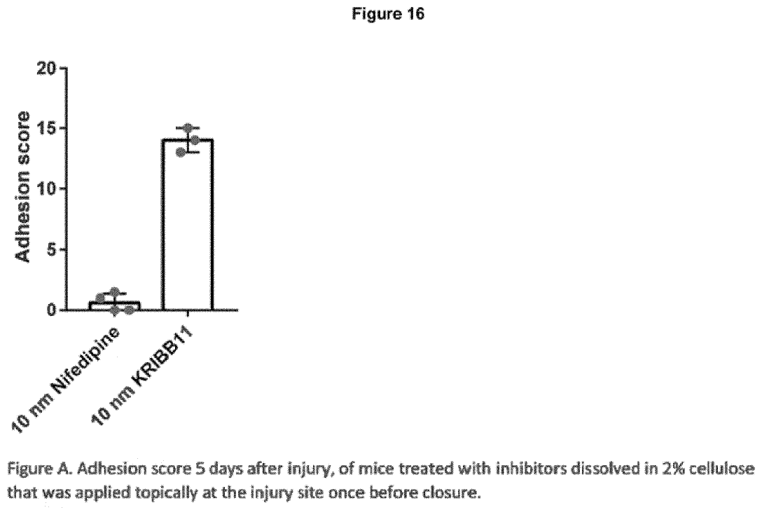

FIG. 16 shows the adhesion score in mice 5 days after injury. While a single application of 10 nM Nifedipine prevents adhesion, 10 nM KRIBB11 shows no effect.

Murine Adhesion Model

Mice were anesthetized by an intraperitoneal injection of a Medetomidin (500 μg/kg), Midazolam (5 mg/kg) and Fentanyl (50 μg/kg) cocktail, hereafter referred to as MMF. Monitoring anesthetic depth was assessed by toe reflex. Eyes were covered with Bepanthen to avoid dehydration, and the abdomen was shaved and disinfected with betadine and sterile phosphate buffered saline (PBS). Animals were kept on their backs on a heating plate at 37° C. A midline laparotomy (1-1.5 cm) was performed through the skin and peritoneum. Four hooks, positioned around the incision and fixed to a retractor and magnetic base plate, allowed for clear access to the abdominal cavity. A small surgical brush was used to gently abrade the peritoneal surface and apposing cecal surface. Two surgical knots using 4-0 silk sutures (Ethicon) were then placed through the serosal surface of the peritoneum. A cotton swab was used to gently apply a dab of talc powder (Sigma Aldrich, #243604) onto the injured surfaces. Before closure of the incision, buprenorphine (0.1 mg/kg) was pipetted in the abdomen to allow for initial post-surgical analgesia. For long-term analgesia, metamizol (Novalgin, 200 mg/kg) was provided through daily injection. The peritoneum and skin were closed with two separate 4-0 silk sutures (Ethicon). Upon closure of the incision, mice were woken up by antagonizing the MMF solution through a subcutaneous cocktail injection of Atipamezol (1 mg/kg) and Flumazenil (0.25 mg/kg). Mice were allowed to recover on a heating pad, after which they were housed together (females) or individually (males), and followed for 1-5 days. Adhesions were scored using gross morphological features that indicated adhesion development. Five individual adhesion features were scored (see Extended Table 1) that together provided a cumulative value that determined the total adhesion score. With this system, complete absence of adhesions was scored as 0, whereas the maximum adhesion score was 15.

Localized Treatment with Topical Cellulose

Small molecule inhibitors were solubilized in sterile 2% hydroxyethyl-cellulose (Sigma Aldrich, #09368). Compounds were being added immediately prior to surgery, and were derived from a 100-150 mM stock solution to minimize the final DMSO content. The final solution (200 μL per 30 g body weight) was sandwiched between the visceral and parietal layer of the injured cecum and peritoneum respectively.

### REFERENCES

1. Stangel, J. J., Nisbet, J. D. & Settles, H. Formation and prevention of postoperative abdominal adhesions. J. Reprod. Med. 29, 143-156 (1984).
2. Rocca, A. et al. Prevention and treatment of peritoneal adhesions in patients affected by vascular diseases following surgery: a review of the literature. Open Med. Wars. Pol. 11, 106-114 (2016).
3. Liu, Y. et al. Transition of mesothelial cell to fibroblast in peritoneal dialysis: EMT, stem cell or bystander? Perit. Dial. Int. J. Int. Soc. Perit. Dial. 35, 14-25 (2015).
4. Luijendijk, R. W. et al. Foreign material in postoperative adhesions. Ann. Surg. 223, 242-248 (1996).
5. Suzuki, T. et al. An injured tissue affects the opposite intact peritoneum during postoperative adhesion formation. Sci. Rep. 5, 7668 (2015).
6. Ray, N. F., Denton, W. G., Thamer, M., Henderson, S. C. & Perry, S. Abdominal adhesiolysis: inpatient care and expenditures in the United States in 1994. J. Am. Coll. Surg. 186, 1-9 (1998).
7. Ellis, H. et al. Adhesion-related hospital readmissions after abdominal and pelvic surgery: a retrospective cohort study. Lancet Lond. Engl. 353, 1476-1480 (1999).
8. Lower, A. M. et al. The impact of adhesions on hospital readmissions over ten years after 8849 open gynaecological operations: an assessment from the Surgical and Clinical Adhesions Research Study. BJOG Int. J. Obstet. Gynaecol. 107, 855-862 (2000).
9. Rinkevich, Y. et al. Identification and prospective isolation of a mesothelial precursor lineage giving rise to smooth muscle cells and fibroblasts for mammalian internal organs, and their vasculature. Nat. Cell Biol. 14, 1251-1260 (2012).
10. Schade, D. S. & Williamson, J. R. The pathogenesis of peritoneal adhesions: an ultrastructural study. Ann. Surg. 167, 500-510 (1968).
11. Tsai, J. M. et al. Adhesions are derived from hypoxia responsive MSLN+ mesothelial cells and are resolved via targeted therapies. Sci. Transl. Med. (2018).
12. Carpinteri, S. et al. Experimental study of delivery of humidified-warm carbon dioxide during open abdominal surgery. Br. J. Surg. 105, 597-605 (2018).
13. Boettiger, D. Quantitative measurements of integrin-mediated adhesion to extracellular matrix. Methods Enzymol. 426, 1-25 (2007).
14. Shin, D. & Minn, K. W. The effect of myofibroblast on contracture of hypertrophic scar. Plast. Reconstr. Surg. 113, 633-640 (2004).
15. Bardele, C. F. Comparative study of axopodial microtubule patterns and possible mechanisms of pattern control in the centrohelidian heliozoa Acanthocystis, Raphidiophrys and Heterophrys. J. Cell Sci. 25, 205-232 (1977).
16. Strowitzki, M. J. et al. Pharmacological HIF-inhibition attenuates postoperative adhesion formation. Sci. Rep. 7, 13151 (2017).
17. Clapham, D. E. Calcium signaling. Cell 131, 1047-1058 (2007).
18. Macosko, E. Z. et al. Highly Parallel Genome-wide Expression Profiling of Individual Cells Using Nanoliter Droplets. Cell 161, 1202-1214 (2015).
19. Ziegenhain, C. et al. Comparative Analysis of Single-Cell RNA Sequencing Methods. Mol. Cell 65, 631-643.e4 (2017).
20. Wolf, F. A., Angerer, P. & Theis, F. J. SCANPY: large-scale single-cell gene expression data analysis. Genome Biol. 19, 15 (2018).
21. McDavid, A. et al. Data exploration, quality control and testing in single-cell qPCR-based gene expression experiments. Bioinforma. Oxf. Engl. 29, 461-467 (2013).
22. Robertson, D., Lefebvre, G. & CLINICAL PRACTICE GYNAECOLOGY COMMITTEE. Adhesion prevention in gynaecological surgery. J. Obstet. Gynaecol. Can. JOGC J. Obstet. Gynecol. Can. JOGC 32, 598-602 (2010).
23. Rothman, J. E. Mechanisms of intracellular protein transport. Nature 372, 55-63 (1994).
24. Etienne-Manneville, S. & Hall, A. Rho GTPases in cell biology. Nature 420, 629-635 (2002).
25. Medjkane, S., Perez-Sanchez, C., Gaggioli, C., Sahai, E. & Treisman, R. Myocardin-related transcription factors and SRF are required for cytoskeletal dynamics and experimental metastasis. Nat. Cell Biol. 11, 257-268 (2009).
26. Tao, J., Shumay, E., McLaughlin, S., Wang, H. & Malbon, C. C. Regulation of AKAP-membrane interactions by calcium. J. Biol. Chem. 281, 23932-23944 (2006).
27. Ducros, E. et al. Endothelial protein C receptor expressed by ovarian cancer cells as a possible biomarker of cancer onset. Int. J. Oncol. 41, 433-440 (2012).
28. Keshava, S. et al. Endothelial cell protein C receptor opposes mesothelioma growth driven by tissue factor. Cancer Res. 73, 3963-3973 (2013).
29. Wang, D. et al. Identification of multipotent mammary stem cells by protein C receptor expression. Nature 517, 81-84 (2015).
30. Watkins, S. C. & Salter, R. D. Functional connectivity between immune cells mediated by tunneling nanotubules. Immunity 23, 309-318 (2005).
31. Polak, R., de Rooij, B., Pieters, R. & den Boer, M. L. B-cell precursor acute lymphoblastic leukemia cells use tunneling nanotubes to orchestrate their microenvironment. Blood 126, 2404-2414 (2015).
32. Mutsaers, S. E. et al. Mesothelial cells in tissue repair and fibrosis. Front. Pharmacol. 6, 113 (2015).
33. He, X. et al. Mesothelin promotes epithelial-to-mesenchymal transition and tumorigenicity of human lung cancer and mesothelioma cells. Mol. Cancer 16, 63 (2017).
34. Martinez-Estrada, O. M. et al. Wt1 is required for cardiovascular progenitor cell formation through transcriptional control of Snail and E-cadherin. Nat. Genet. 42, 89-93 (2010).
35. Bhattacharya, R., Mitra, T., Ray Chaudhuri, S. & Roy, S. S. Mesenchymal splice isoform of CD44 (CD44s) promotes EMT/invasion and imparts stem-like properties to ovarian cancer cells. J. Cell. Biochem. 119, 3373-3383 (2018).
36. Ertürk, A. et al. Three-dimensional imaging of solvent-cleared organs using 3DISCO. Nat. Protoc. 7, 1983-1995 (2012).
37. Arganda-Carreras, I. et al. Trainable Weka Segmentation: a machine learning tool for microscopy pixel classification. Bioinforma. Oxf. Engl. 33, 2424-2426 (2017).
38. Steger, C. An Unbiased Detector of Curvilinear Structures. IEEE Trans. PATTERN Anal. Mach. Intell. 20, 13 (1998).
39. Dobin, A. et al. STAR: ultrafast universal RNA-seq aligner. Bioinforma. Oxf. Engl. 29, 15-21 (2013).
40. Schiller, H. B. et al. Time- and compartment-resolved proteome profiling of the extracellular niche in lung injury and repair. Mol. Syst. Biol. 11, 819 (2015).
41. Kramer, A., Green, J., Pollard, J. & Tugendreich, S. Causal analysis approaches in Ingenuity Pathway Analysis. Bioinforma. Oxf. Engl. 30, 523-530 (2014).
42. Wickham, H. ggplot2: Elegant Graphics for Data Analysis. (Springer-Verlag, 2009).
43. R Development Core Team. R: A Language and Environment for Statistical Computing.

1. A compound for use in a method of reducing the formation of heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.

2. The compound for the use of claim 1, wherein a mesothelial cell is activated by hypoxia, ischemia, inflammation, infection, a chemical stimulus, desiccation, a mechanical trauma, cold shock, heat shock, osmotic shock or a foreign body to become an activated mesothelial cell.

3. The compound for the use of any one of the preceding claims, wherein a heliocyte is characterized by membrane protrusions of the akropodia-type, and/or membrane protrusions of the filopodia-type.

4. The compound for the use of any one of the preceding claims, wherein a heliocyte is characterized by vesicle and/or exosome secretion.

5. The compound for the use of any one of the preceding claims, wherein heliocytes develop adhesions.

6. The compound for the use of any one of the preceding claims, wherein the development of adhesions by heliocytes results in adhesiogenesis.

7. The compound for the use of claim 6, wherein adhesiogenesis is inter- or intra-organ adhesiogenesis.

8. The compound for the use of any one of the claims 5 to 7, wherein inter- or intra-organ adhesiogenesis occurs postoperative.

9. The compound for the use of any one of the preceding claims, wherein the compound is capable of preventing the transmission of a mesothelial cell to the pathogenic phenotype of a heliocyte and/or capable of inducing apoptosis in a heliocyte, but not in a mesothelial cell.

10. The compound for the use of any one of the preceding claims, wherein the compound blocks cytoskeletal remodeling, blocks protein trafficking, blocks calcium signaling, or blocks heat shock protein signaling.

11. The compound for the use of any one of the preceding claims, wherein the compound is selected from the group consisting of Bepridil, Verapamil, Diltiazem, Nifedipine, Rhosin, CK-666, Golgicide A, KNK437, and Quercetin.

12. The compound for the use of claim 10 or 11, wherein the compound is further combined with a heat shock protein signaling blocker, preferably Quercetin.

13. The compound for the use of claim 11, wherein the compound is a calcium channel blocker selected from the group consisting of Diltiazem, Verapamil, Nifedipine and Bepridil.

14. The compound for the use of claim 11, wherein the compound is a heat shock protein signaling blocker selected from the group consisting of KNK437, and Quercetin.

15. The compound for the use of claim 11, wherein the compound is a cytoskeletal remodeling blocker selected from the group consisting of Rhosin, and CK-666.

16. The compound for the use of any one of the preceding claims, wherein the use comprises administering the compound of any one of the claims 10 to 15 after surgery or injury, determining the adhesion formation by heliocytes and continuing the compound treatment if the adhesion formation by heliocytes decreased as compared to the pre-treatment.

17. An in vitro bead assay for analyzing heliocytes and/or the formation of adhesions, comprising the steps of
a) seeding mesothelial cells onto a coated dish and letting said cells grow to a monolayer;
b) coating carrier beads with mesothelial cells;
c) activating said cells coated on said carrier beads of step b) with a stimulus,
d) seeding said activated cells coated on said carrier beads of step b) and c) onto the monolayer of step a);
and analyzing the activated mesothelial cells on said carrier beads, or analyzing the activated mesothelial cells eluted from said carrier beads.

18. The in vitro bead assay of claim 17, wherein the mesothelial cells are preferably Met-5A positive, before seeded in step a) and/or coated in step b).

19. The in vitro bead assay of claim 17, wherein the activated cells on said carrier beads of step c) and/or step d) are capable of fusing the cell-coated beads together and can optionally be selected by size.

20. The in vitro bead assay of claim 17, wherein the stimulus of step c) is selected from the group consisting of hypoxia, ischemia, inflammation, infection, a chemical stimulus, desiccation, a mechanical trauma, cold shock, heat shock, osmotic shock, or a foreign body.

21. The in vitro bead assay of claims 17 to 20, further comprises
i) contacting said activated cells coated on said carrier beads after step c) with a compound; and
ii) determining the effect of the compound on the activated mesothelial cells and/or formation of adhesions after step d).

22. The compound for the use of claim 9, wherein said capability of said compound is determined by the in vitro bead assay of claims 17 to 21.

23. The in vitro bead assay of claims 17 to 21 in use for determining the capability of a compound to
a) prevent the transmission of a mesothelial cell to the pathogenic phenotype of a heliocyte; and/or
b) induce apoptosis in a heliocyte, but not in a mesothelial cell; and/or
c) prevent the formation of adhesion and/or adhesiogenesis.

24. An in vitro method for determining the formation of heliocytes, wherein the method comprises obtaining a sample comprising mesothelial cells from a subject, preparing the sample according to the in vitro bead assay of claims 17 to 21 and determining the formation of heliocytes in said in vitro bead assay.

25. An in vitro method for treating heliocytes and/or adhesions formed by heliocytes, wherein the method comprises obtaining a sample comprising mesothelial cells from a subject, preparing the sample according to the in vitro bead assay of claims 17 to 20 and treating the heliocytes and/or adhesions formed by heliocytes by contacting said heliocytes with a compound according to claim 21.

26. A calcium channel blocker for use in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.

27. The calcium channel blocker for the use of claim 26, wherein the calcium channel blocker is Diltiazem, Verapamil, Nifedipine and Bepridil, preferably Diltiazem, Verapamil, Bepridil.

28. The calcium channel blocker for the use of claims 26 and 27, wherein the method comprises
a) administering to a subject an effective amount of calcium channel blocker to prevent the formation of adhesion by heliocytes after surgery or injury;
b) determining the formation of adhesion by heliocytes after the treatment with said calcium channel blocker;
c) continuing the treatment if the heliocytes and/or adhesion formation by heliocytes decreased as compared to the pre-treatment.

29. A heat shock protein signaling blocker for use in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain.

30. The heat shock protein signaling blocker for the use of claim 29, wherein the the heat shock protein signaling blocker is KNK437, or Quercetin, preferably KNK437.

31. The heat shock protein signaling blocker for the use of claims 29 and 30, wherein the method comprises
a) administering to a subject an effective amount of heat shock protein signaling blocker to prevent the formation of adhesion by heliocytes after surgery or injury;
b) determining the formation of adhesion by heliocytes after the treatment with said heat shock protein signaling blocker;
c) continuing the treatment if the heliocytes and/or adhesion formation by heliocytes decreased as compared to the pre-treatment.

32. A cytoskeletal remodeling blocker for use in a method of reducing adhesions formed by heliocytes, wherein the heliocyte is an activated mesothelial cell, and wherein the activated mesothelial cell is, in comparison to a non-activated mesothelial cell, characterized by increased expression of Rho, ARF-GAP1, AKAP12, HSP70, HSP27, HSP105 on protein level and/or a phosphorylated Myosin 9 light chain, preferably Rhosin.

33. The cytoskeletal remodeling blocker for the use of claim 32, and wherein the cytoskeletal remodeling blocker is Rhosin, or CK-666, preferably Rhosin.

34. The cytoskeletal remodeling blocker for the use of claims 32 and 33, wherein the method comprises
a) administering to a subject an effective amount of cytoskeletal remodeling blocker to prevent the formation of adhesion by heliocytes after surgery or injury;
b) determining the formation of adhesion by heliocytes after the treatment with said cytoskeletal remodeling blocker;
c) continuing the treatment if the heliocytes and/or adhesion formation by heliocytes decreased as compared to the pre-treatment.

35. A pharmaceutical composition for use in a method of reducing the formation of heliocytes, comprising at least one compound(s) of claims 10 to 15 and one or more pharmaceutically acceptable excipients.

36. An in vitro method for detecting the presence of heliocytes forming adhesions in a subject, comprising:
a) providing a sample obtained from a subject, said sample comprising one or more cell(s);
b) seeding a plurality of cells of a subject in the in vitro bead assay of claims 17 to 20;
c) contacting said cells with
i) the compound according to claims 10 to 15 and/or,
ii) the pharmaceutical composition according to claim 35; and
c) detecting the presence of heliocytes forming adhesions in the cells seeded in said in vitro bead assay, wherein the detection of heliocytes forming adhesions is indicative of heliocytes forming adhesions in the subject.

37. A method of selecting a subject for calcium channel blocker treatment, comprising
a) determining the heliocyte formation in a sample, wherein the sample has been obtained from a subject prior to calcium channel blocker treatment;
b) determining the heliocyte formation in a sample, wherein the sample has been obtained from the subject after treatment with the calcium channel blocker;
c) selecting the subject for continuing the calcium channel blocker treatment if the heliocyte formation is decreased in step c) as compared to step a).

38. A method of selecting a subject for heat shock protein signaling blocker treatment, comprising
a) determining the heliocyte formation in a sample, wherein the sample has been obtained from a subject prior to heat shock protein signaling blocker treatment;
b) determining the heliocyte formation in a sample, wherein the sample has been obtained from the subject after treatment with the heat shock protein signaling blocker;
c) selecting the subject for continuing the heat shock protein signaling blocker treatment if the heliocyte formation is decreased in step c) as compared to step a).

39. A method of selecting a subject for cytoskeletal remodeling blocker treatment, comprising
a) determining the heliocyte formation in a sample, wherein the sample has been obtained from a subject prior to cytoskeletal remodeling blocker treatment;
b) determining the heliocyte formation in a sample, wherein the sample has been obtained from the subject after treatment with the cytoskeletal remodeling blocker;
c) selecting the subject for continuing the cytoskeletal remodeling blocker treatment if the heliocyte formation is decreased in step c) as compared to step a).


SEQUENCE LISTING (1 sequence)

SEQ ID NO: 1 - DNA, 37 bp, artificial sequence
  primer
gcctgtccgcggaagcagtggtatcaacgcagagtac